(19) United States
(12) Patent Application Publication
(10) Pub. No.: US 2025/0136689 A1
(43) Pub. Date: May 1, 2025

(54) COMPOSITIONS FOR AND METHODS OF TREATING HEMATOLOGICAL CANCERS

(71) Applicant: Duke University, Durham, NC (US)

(72) Inventors: Fang Li, Durham, NC (US); Chuan-Yuan Li, Durham, NC (US)

(21) Appl. No.: 18/835,479

(22) PCT Filed: Feb. 3, 2023
(86) PCT No.: PCT/US2023/061925

§ 371 Date: Aug. 2, 2024

Related U.S. Application Data
(60) Provisional application No. 63/306,613, filed on Feb. 4, 2022.

(51) Int. Cl. C07K 16/28; A61K 40/11; A61K 40/15; A61K 40/17; A61K 40/31; A61K 40/41; A61K 45/06; A61K 47/68; A61P 35/02; C07K 16/44; C12N 15/86

(52) U.S. Cl. CPC C07K 16/2809; A61K 40/11; A61K 40/15; A61K 40/17; A61K 40/31; A61K 40/4285; A61K 45/06; A61K 47/6879; A61P 35/02; C07K 16/44; C12N 15/86; C07K 2317/31; C07K 2317/52; C07K 2317/569; C07K 2317/622

(57) ABSTRACT

Disclosed herein are compositions comprising a chimeric fusion protein targeting phosphatidylserine on the surface of hematological cancer cells and methods of using the compositions to treat a hematological cancer in a subject.

### CROSS-REFERENCE TO RELATED APPLICATIONS

This application claims priority to U.S. Provisional Application No. 63/306,613 filed 4 Feb. 2022, which is incorporated herein in its entirety.

### STATEMENT REGARDING FEDERALLY SPONSORED RESEARCH OR DEVELOPMENT

This invention was made with government support under CA216876 awarded by the National Institutes of Health. The government has certain rights in the invention.

### REFERENCE TO THE SEQUENCE LISTING

The Sequence Listing submitted 3 Feb. 2023 as an XML file named ‘23-2063-WO’, created on 3 Feb. 2023 and having a size of 1 MB is hereby incorporated by reference pursuant to 37 C.F.R. § 1.52(e)(5).

### BACKGROUND

In cancer treatment, it is highly desirable to exploit cancer-specific markers for therapeutic development. Many such markers are cell surface markers. Phosphatidylserine (PS) is an abundant lipid molecule and an integral part of the cellular membrane. Unlike other abundant lipid molecules that evenly distribute in both the outer and inner cellular membranes, phosphatidylserine normally only resides in the inner cellular membrane. PS often ‘flips’ from the inner to the outer cellular membrane in dying cells, especially in apoptotic cells. As a result, cell surface levels of PS have become the most widely used molecular marker to quantify cellular apoptosis in research. Most of the current paradigms for cancer therapy assumes that PS-expressing tumor cells, either from exposure to internal or external stressors, are destined to die from apoptosis. However, PS-expressing tumor cells may survive the activation of the apoptosis cascade. Thus, there remains an unmet medical need for ensuring the complete cell death of PS-expressing cancer cells including hematologic cancer cells as well as an unmet medical need for enhancing the efficacy of cancer therapies and/or treatments.

### BRIEF DESCRIPTION OF THE FIGURES

FIG. 1A-FIG. 1C show the survival of phosphatidylserine-expressing AML cells. FIG. 1A shows FACS sorting of untreated c1498 cells (in DMEM with 8% FBS). The rectangles indicate the sorted cells. FIG. 1B shows FACS sorting of c1498 cells treated with cytarabine (1 M) for 24 hrs). FIG. 1C shows the estimated live cell numbers in the sorted cells. About 1000 each of the sorted cells were placed into multi-well plates. When the cells reached sufficient numbers, they were counted at different time points to measure their growth rates. The growth rates were then used to estimate the initial live cell numbers. The following formula was used to calculate the minimal initial live cell numbers: Minimal initial cell number=Total cell population/(growth rate*t)(days to reach the current cell number).

FIG. 2A-FIG. 2B shows phagocytosis KG1a cells by macrophages. FIG. 2A shows the representative fluorescence images of phagocytosis assay. THP1 cells transduced with mCherry were differentiated into macrophages following previously established procedures. The macrophages were then incubated with non-treated or doxorubicin-treated KG1a human AML cell line transduced with EGFP in the presence of either isotype control (IgG1) or AnnexinV-IgG1Fc. After 2 hrs of incubation, the macrophages and the KG1a cells they have engulfed were counted. FIG. 2B shows quantitative estimates of phagocytosis of KG1a cells by THP1-derived macrophages. the phagocytic index is calculated as: (Average number of KG1a cells engulfed per macrophage)×(percentage of macrophages which have engulfed KG1a cells)×200.

FIG. 3 shows anti-tumor efficacy of Annexin V-Fc in the c1498. About 2×10⁵ c1498 cells were injected into the tail vein of C57BL/6 mice. At 24 hrs after the injection, the mice were treated with 100 mg/kg cytarabine (injected i.p. every 12 hrs for 3 times). At 24 hrs after the last treatment, Annexin V-Fc was given i.p. 4 times at 72 hours intervals. The mice were then observed and sacrificed until they were moribund. Shown were the survival of different mice after they were treated with different antibody and drug combinations.

FIG. 4 shows the tumor cell-killing efficacy of AnnexinV-OKT3 in MV4-11 human AML cells. Luciferase-labeled MV4-11 AML cells were mixed with PBMC cells at a ratio of 1:10. Bispecific proteins AnnexinV-scFv(OKT3)-Fc and AnnexinV-scFv(OKT3)-Hinge were added to the mixed cell population at different concentrations in 96-well plates. The survival of the MV4-11 cells were then quantified by measuring the luciferase activities of individual wells at 24 hrs post bispecific protein addition (n=3).

FIG. 5A-FIG. 5H shows different versions of the Annexin V-scFv (αCD3) bispecific fusion protein. FIG. 5A shows an AnnexinV-αCD3 BITE fusion protein. FIG. 5B shows an (AnnexinV-scFv(αCD3) BITE-Fc)₂ fusion protein. FIG. 5C shows an (AnnexinV-scFv(αCD3)-Hinge)₂ fusion protein. FIG. 5D shows an (AnnexinV-Fc):(scFv(αCD3)-Fc) fusion protein. FIG. 5E shows an AnnexinV-scFv(αCD3))-Fc:Fc fusion protein. FIG. 5F shows an (Annexin V-Fc-SIRPα)₂ fusion protein. FIG. 5G show an (AnnexinV-SIRPα-Fc)₂ fusion protein. FIG. 5H shows an (AnnexinV-Fc-NKG2D)₂ fusion protein. In D&E, Fc domains have the knobs-in-holes structure to facilitate heterodimerization.

### BRIEF SUMMARY

Disclosed herein is a chimeric fusion protein comprising a phosphatidylserine (PS) binding domain operably linked an immunostimulatory domain. Disclosed herein is a chimeric fusion protein comprising a phosphatidylserine (PS) binding domain operably linked to an immunostimulatory domain, wherein the immunostimulatory domain further comprises a conjugated small molecule. Disclosed herein is a chimeric fusion protein comprising a secretion signal and a phosphatidylserine (PS) binding domain operably linked an immunostimulatory domain. Disclosed herein is a chimeric fusion protein comprising a secretion signal and a phosphatidylserine (PS) binding domain operably linked to an immunostimulatory domain, wherein the immunostimulatory domain further comprises a conjugated small molecule. Disclosed herein is a chimeric fusion protein comprising a PS-binding domain operably linked to an IgG1 Fc domain and an additional immunostimulatory domain. Disclosed herein is a chimeric fusion protein comprising a PS-binding domain operably linked to an IgG1 Fc domain and an additional immunostimulatory domain, wherein the additional immunostimulatory domain comprises the IgV domain of SIRPα or the extracellular domain of NKG2D protein. Disclosed herein is a chimeric fusion protein comprising a PS-binding domain operably linked to a single-chain antibody (scFV) to a human CD3 protein. Disclosed herein is a chimeric fusion protein comprising an Annexin-binding domain operably linked to a single-chain antibody (scFV) to a human CD3 protein. Disclosed herein is a chimeric fusion protein comprising a PS-binding domain operably linked to a single-chain antibody (scFV) to a human CD3 protein and one or more additional immunostimulatory domains. Disclosed herein is a chimeric fusion protein comprising an Annexin-binding domain operably linked to a single-chain antibody (scFV) to a human CD3 protein and one or more additional immunostimulatory domains.

Disclosed herein is an antibody-drug conjugate comprising a disclosed chimeric fusion protein comprising a phosphatidylserine (PS) binding domain. Disclosed herein is an antibody-drug conjugate comprising a disclosed chimeric fusion protein comprising a phosphatidylserine (PS) binding domain and an immunostimulatory domain. Disclosed herein is an antibody drug conjugate comprising a disclosed chimeric fusion protein comprising a phosphatidylserine (PS) binding domain operably linked to an immunostimulatory domain, wherein the immunostimulatory domain further comprises a conjugated small molecule. Disclosed herein is an antibody-drug conjugate comprising a disclosed chimeric fusion protein comprising a secretion signal and a phosphatidylserine (PS) binding domain. Disclosed herein is an antibody-drug conjugate comprising a disclosed chimeric fusion protein comprising a secretion signal, a phosphatidylserine (PS) binding domain, and an immunostimulatory domain. Disclosed herein is an antibody drug conjugate comprising a disclosed chimeric fusion protein comprising a secretion signal and a phosphatidylserine (PS) binding domain operably linked to an immunostimulatory domain, wherein the immunostimulatory domain further comprise a conjugated small molecule. Disclosed herein is an antibody-drug conjugate comprising a disclosed chimeric fusion protein comprising a PS-binding domain operably linked to an IgG1 Fc domain. Disclosed herein is an antibody-drug conjugate comprising a disclosed chimeric fusion protein comprising a PS-binding domain operably linked to an IgG1 Fc domain and an additional immunostimulatory domain. Disclosed herein is an antibody-drug conjugate comprising a disclosed chimeric fusion protein comprising a PS-binding domain operably linked to an IgG1 Fc domain and an additional immunostimulatory domain, wherein the additional immunostimulatory domain comprises the IgV domain of SIRPα or the extracellular domain of NKG2D protein. Disclosed herein is an antibody-drug conjugate comprising a disclosed chimeric fusion protein comprising a PS-binding domain operably linked to a single-chain antibody (scFV) to a human CD3 protein. Disclosed herein is an antibody-drug conjugate comprising a disclosed chimeric fusion protein comprising an Annexin-binding domain operably linked to a single-chain antibody (scFV) to a human CD3 protein.

Disclosed herein is an isolated nucleic acid molecule comprising a nucleic acid sequence encoding a chimeric fusion protein comprising a phosphatidylserine (PS) binding domain. Disclosed herein is an isolated nucleic acid molecule comprising a nucleic acid sequence encoding a chimeric fusion protein comprising a phosphatidylserine (PS) binding domain and an immunostimulatory domain. Disclosed herein is an isolated nucleic acid molecule comprising a nucleic acid sequence encoding a chimeric fusion protein comprising a phosphatidylserine (PS) binding domain operably linked an immunostimulatory domain. Disclosed herein is an isolated nucleic acid molecule comprising a nucleic acid sequence encoding a phosphatidylserine (PS) binding domain, a linker, and an immunostimulatory domain. Disclosed herein is an isolated nucleic acid molecule comprising a nucleic acid sequence encoding a chimeric fusion protein comprising a phosphatidylserine (PS) binding domain operably linked to an immunostimulatory domain, wherein the immunostimulatory domain further comprise a conjugated small molecule.

Disclosed herein is a vector comprising an isolated nucleic acid molecule comprising a nucleic acid sequence encoding a chimeric fusion protein comprising a phosphatidylserine (PS) binding domain and an immunostimulatory domain. Disclosed herein is a vector comprising an isolated nucleic acid molecule comprising a nucleic acid sequence encoding a chimeric fusion protein comprising a phosphatidylserine (PS) binding domain operably linked an immunostimulatory domain. Disclosed herein is a vector comprising an isolated nucleic acid molecule comprising a nucleic acid sequence encoding a chimeric fusion protein comprising a phosphatidylserine (PS) binding domain operably linked to an immunostimulatory domain, wherein the immunostimulatory domain further comprise a conjugated small molecule. Disclosed herein is a vector comprising an isolated nucleic acid molecule comprising a nucleic acid sequence encoding a chimeric fusion protein comprising a secretion signal and a phosphatidylserine (PS) binding domain operably linked an immunostimulatory domain. Disclosed herein is a vector comprising an isolated nucleic acid molecule comprising a nucleic acid sequence encoding a chimeric fusion protein comprising a secretion signal, a phosphatidylserine (PS) binding domain, a linker, and an immunostimulatory domain.

Disclosed herein is a method of treating a hematological cancer, the method comprising treating a subject in need thereof by administering to the subject in need thereof a therapeutically effective amount of a disclosed chimeric fusion protein or a pharmaceutical formulation thereof. Disclosed herein is a method of treating a hematological cancer, the method comprising treating a subject in need thereof by administering to the subject in need thereof a therapeutically effective amount of a disclosed chimeric fusion protein or a pharmaceutical formulation thereof. Disclosed herein is a method of treating a hematological cancer, the method comprising treating a subject in need thereof by administering to the subject in need thereof a therapeutically effective amount of a disclosed chimeric fusion protein targeting phosphatidylserine (PS) or a pharmaceutical formulation thereof. Disclosed herein is a method of treating a hematological cancer, the method comprising treating a subject in need thereof by administering to the subject in need thereof a therapeutically effective amount of a disclosed chimeric fusion protein targeting phosphatidylserine-expressing tumor cells or a pharmaceutical formulation thereof.

Disclosed herein is a method of improving the efficacy of cancer therapy, the method comprising treating a subject in need thereof by administering to the subject in need thereof a therapeutically effective amount of one or more cells transduced with a disclosed chimeric fusion protein or a pharmaceutical formulation thereof. Disclosed herein is a method of improving the efficacy of cancer therapy, the method comprising treating a subject in need thereof by administering to the subject in need thereof a therapeutically effective amount of a disclosed chimeric fusion protein or a pharmaceutical formulation thereof. Disclosed herein is a method of improving the efficacy of cancer therapy, the method comprising treating a subject in need thereof by administering to the subject in need thereof a therapeutically effective amount of a disclosed chimeric fusion protein targeting phosphatidylserine (PS) or a pharmaceutical formulation thereof.

Disclosed herein is a method of improving the efficacy of cancer therapy, the method comprising treating a subject in need thereof by administering to the subject in need thereof a therapeutically effective amount of a disclosed chimeric fusion protein targeting phosphatidylserine-expressing tumor cells or a pharmaceutical formulation thereof.

### DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase ‘consisting essentially of’ limits the scope of a claim to the recited components in a composition or the recited steps in a method as well as those that do not materially affect the basic and novel characteristic or characteristics of the claimed composition or claimed method. The phrase ‘consisting of’ excludes any component, step, or element that is not recited in the claim. The phrase ‘comprising’ is synonymous with ‘including’, ‘containing’, or ‘characterized by’, and is inclusive or open-ended. ‘Comprising’ does not exclude additional, unrecited components or steps.

As used herein, when referring to any numerical value, the term ‘about’ means a value falling within a range that is +10% of the stated value.

Ranges can be expressed herein as from ‘about’ one particular value, and/or to ‘about’ another particular value. When such a range is expressed, a further aspect includes from the one particular value and/or to the other particular value. Similarly, when values are expressed as approximations, by use of the antecedent ‘about,’ it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as ‘about’ that particular value in addition to the value itself. For example, if the value ‘10’ is disclosed, then ‘about 10’ is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms ‘optional’ or ‘optionally’ means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term ‘subject’ refers to the target of administration, e.g., a human being. The term ‘subject’ also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have cancer, be suspected of having cancer, or be at risk of developing cancer.

As used herein, the term ‘diagnosed’ means having been subjected to an examination by a person of skill, for example, a physician, and found to have a condition that can be diagnosed or treated by one or more of the disclosed chimeric fusion protein, the disclosed isolated nucleic acid molecules, the disclosed vectors, the disclosed cells, the disclosed pharmaceutical formulations, or a combination thereof, or by one or more of the disclosed methods. For example, ‘diagnosed with a disease or disorder’ means having been subjected to an examination by a person of skill, for example, a physician, and found to have a condition (such as a hematological cancer) that can be treated by one or more of the disclosed chimeric fusion protein, the disclosed isolated nucleic acid molecules, the disclosed vectors, the disclosed cells, the disclosed pharmaceutical formulations, or a combination thereof, or by one or more of the disclosed methods. For example, “suspected of having a disease or disorder” can mean having been subjected to an examination by a person of skill, for example, a physician, and found to have a condition (such as a hematological cancer) that can likely be treated by one or more of the disclosed chimeric fusion protein, the disclosed isolated nucleic acid molecules, the disclosed vectors, the disclosed cells, the disclosed pharmaceutical formulations, or a combination thereof, or by one or more of the disclosed methods. In an aspect, an examination can be physical, can involve various tests (e.g., blood tests, genotyping, biopsies, etc.), scans (e.g., CT scans, PET scans, etc.), and assays (e.g., enzymatic assay), or a combination thereof.

A “patient” refers to a subject afflicted with a disease or disorder (e.g., a hematological cancer). In an aspect, a patient can refer to a subject that has been diagnosed with or is suspected of having a disease or disorder such as a hematological cancer. In an aspect, a patient can refer to a subject that has been diagnosed with or is suspected of having a disease or disorder and is seeking treatment or receiving treatment for a disease or disorder (such as hematological cancer).

As used herein, the phrase “identified to be in need of treatment for a disease or disorder,” or the like, refers to selection of a subject based upon need for treatment of the disease or disorder. For example, a subject can be identified as having a need for treatment of a disease or disorder (e.g., a hematological cancer) based upon an earlier diagnosis by a person of skill and thereafter subjected to treatment for the cancer. In an aspect, the identification can be performed by a person different from the person making the diagnosis. In an aspect, the administration can be performed by one who performed the diagnosis.

As used herein, “activated” and “activation” can refer to the state of a T cell that has been sufficiently stimulated to induce detectable cellular proliferation. Activation can also be associated with induced cytokine production and detectable effector functions. The term “activated T cells” can refer to T cells that are proliferating. Signals generated through the TCR alone may be insufficient for full activation of the T cell and one or more secondary or costimulatory signals may also be required. Thus, T cell activation comprises a primary stimulation signal through the TCR/CD3 complex and one or more secondary costimulatory signals. Costimulation can be evidenced by proliferation and/or cytokine production by T cells that have received a primary activation signal, such as stimulation through the TCR/CD3 complex.

As used herein, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having received one or more of the disclosed chimeric fusion proteins, the disclosed isolated nucleic acid molecules, the disclosed vectors, the disclosed cells, the disclosed pharmaceutical formulations, or a combination thereof). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to a native or control level (e.g., a subject not having received one or more of the disclosed chimeric fusion proteins, the disclosed isolated nucleic acid molecules, the disclosed vectors, the disclosed cells, the disclosed pharmaceutical formulations, or a combination thereof). In an aspect, the inhibition or reduction can be 0-25%, 25-50%, 50-75%, or 75-100% as compared to native or control levels. In an aspect, a native or control level can be a pre-disease or pre-disorder level (such as a pre-cancer state).

The words “treat” or “treating” or “treatment” include palliative treatment, that is, treatment designed for the relief of symptoms rather than the curing of the disease, pathological condition, or disorder (e.g., a hematological cancer); preventative treatment, that is, treatment directed to minimizing or partially or completely inhibiting the development of the associated disease, pathological condition, or disorder (e.g., a hematological cancer); and supportive treatment, that is, treatment employed to supplement another specific therapy directed toward the improvement of the associated disease, pathological condition, or disorder (e.g., a hematological cancer). In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a disease or disorder can reduce the severity of an established a disease or disorder in a subject by 1%-100% as compared to a control (such as, for example, an individual not having cancer). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or disorder (e.g., a hematological cancer). For example, treating a disease or disorder can reduce one or more symptoms of a disease or disorder in a subject by 1%-100% as compared to a control (such as, for example, an individual not having cancer). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or disorder (e.g., a hematological cancer). It is understood that treatment does not necessarily refer to a cure or complete ablation or eradication of a disease or disorder. However, in an aspect, treatment can refer to a cure or complete ablation or eradication of a disease or disorder (such as a hematological cancer).

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a disease or disorder having chromatin deregulation and/or chromatin dysregulation is intended. The words “prevent”, “preventing”, and “prevention” also refer to prophylactic or preventative measures for protecting or precluding a subject (e.g., an individual) not having a given a disease or disorder (such as a hematological cancer) or related complication from progressing to that complication. In an aspect, preventing metastasis is intended.

As used herein, the terms “administering” and “administration” refer to any method of providing one or more of the disclosed chimeric fusion proteins, the disclosed isolated nucleic acid molecules, the disclosed vectors, the disclosed cells, the disclosed pharmaceutical formulations, or a combination thereof, or by one or more of the disclosed methods to a subject. Such methods are well known to those skilled in the art and include, but are not limited to, the following: oral administration, transdermal administration, administration by inhalation, nasal administration, topical administration, in utero administration, intratumoral administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of one or more of the disclosed chimeric fusion proteins, the disclosed isolated nucleic acid molecules, the disclosed vectors, the disclosed cells, the disclosed pharmaceutical formulations, or a combination thereof can comprise administration directly into the CNS or the PNS. Administration can be continuous or intermittent. Administration can comprise a combination of one or more routes.

In an aspect, the skilled person can determine an efficacious dose, an efficacious schedule, and an efficacious route of administration one or more of the disclosed chimeric fusion proteins, the disclosed isolated nucleic acid molecules, the disclosed vectors, the disclosed cells, the disclosed pharmaceutical formulations, or a combination thereof to treat or prevent a disease or disorder (such as a hematological cancer). In an aspect, the skilled person can also alter, change, or modify an aspect of an administering step to improve efficacy of one or more of the disclosed chimeric fusion proteins, the disclosed isolated nucleic acid molecules, the disclosed vectors, the disclosed cells, the disclosed pharmaceutical formulations, or a combination thereof.

By “determining the amount” is meant both an absolute quantification of a particular analyte (e.g., biomarker for hematological cancer, for example) or a determination of the relative abundance of a particular analyte (e.g., a hematological cancer biomarker). The phrase includes both direct or indirect measurements of abundance or both.

As used herein, “modifying the method” can comprise modifying or changing one or more features or aspects of one or more steps of a disclosed method. In an aspect, a method can be altered by changing the amount of the one or more of the disclosed chimeric fusion proteins, the disclosed isolated nucleic acid molecules, the disclosed vectors, the disclosed cells, the disclosed pharmaceutical formulations, or a combination thereof administered to a subject, or by changing the frequency of administration of the one or more of the disclosed chimeric fusion proteins, the disclosed isolated nucleic acid molecules, the disclosed vectors, the disclosed cells, the disclosed pharmaceutical formulations, or a combination thereof to a subject, by changing the duration of time that one or more of the disclosed chimeric fusion proteins, the disclosed isolated nucleic acid molecules, the disclosed vectors, the disclosed cells, the disclosed pharmaceutical formulations, or a combination thereof is administered to a subject, or by substituting for one or more of the disclosed components and/or reagents with a similar or equivalent component and/or reagent. The same applies to all disclosed chimeric fusion proteins, disclosed isolated nucleic acid molecules, disclosed vectors disclosed cells, disclosed pharmaceutical formulations, and any combination thereof.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

A “transposon” is a mobile genetic element that efficiently moves between vectors and chromosomes using the “cut and paste” or “copy and paste” mechanism. During transposase transposition (for example, PB transposase in the PiggyBac transposon system) recognizes transposon-specific sequences of inverted terminal repeats (ITRs) located at both ends of the transposon (there are 5′- and 3′-ITRs in any transposon system), it moves the contents from source sites and embeds them in chromosomal sites, such as TTAA chromosomal sites. In an aspect, the powerful activity of the PiggyBac transposon system makes it easy to transfer genes of interest located between two ITRs to target genomes. The transposon can be divided into Class I transposon (retrotransposon) and Class II transposon (DNA transposon). In Class I transposon, after RNA is transcribed from nucleic acid in a cell or from transposon DNA on the animal genome, the DNA reverse-transcribed from the RNA is transferred to another location on the animal genome. It works by inserting it. Class II transposon cuts nucleic acid in cells or transposon DNA on the animal genome, and then inserts the cut transposon DNA into another location on the animal genome. The Class II transposon may include a first polynucleotide at a 5′ end, a second polynucleotide at a 3′ end, and a third polynucleotide. The first polynucleotide and the second polynucleotide may include an inverted terminal repeat (ITR) sequence. The third polynucleotide may be located between the first polynucleotide and the second polynucleotide. The third polynucleotide may include an exo-polynucleotide. The third polynucleotide may include a polynucleotide encoding a transposase. Unless otherwise specified below, the term “transposon” assumes the case of Class II transposon, but even if the term “transposon” is interpreted as Class I transposon, it is technically If there is no problem, it will not be necessary to limit the interpretation to Class II transposon.

As used herein, “concurrently” means (1) simultaneously in time, or (2) at different times during the course of a common treatment schedule.

The term “contacting” as used herein refers to bringing one or more of the disclosed chimeric fusion proteins, the disclosed isolated nucleic acid molecules, the disclosed vectors, the disclosed cells, the disclosed pharmaceutical formulations, the disclosed anti-chemokines, the disclosed anti-cancer agents, the disclosed chemotherapeutics, or any combination thereof together with a target area or intended target area in such a manner that the disclosed chimeric fusion proteins, the disclosed isolated nucleic acid molecules, the disclosed vectors, the disclosed cells, the disclosed pharmaceutical formulations, the disclosed anti-chemokines, the disclosed anti-cancer agents, the disclosed chemotherapeutics, or any combination thereof can exert an effect on the intended target or targeted area either directly or indirectly. A target area or intended target area can be one or more of a subject's organs (e.g., lungs, heart, liver, kidney, brain, etc.) hosting cancerous cells. In an aspect, a target area or intended target area can be any cell or any organ infected by a disease or disorder (such as a hematological cancer). In an aspect, a target area or intended target area can be any organ, tissue, or cells that are affected by a disease or disorder (such as a hematological cancer). In an aspect, a target or intended target can be a blood-borne cancer or a hematologic cancer.

As used herein, “determining” can refer to measuring or ascertaining the presence and severity of a disease or disorder, such as, for example, a hematologic cancer. Methods and techniques used to determine the presence and/or severity of a disease or disorder are typically known to the medical arts. For example, the art is familiar with the ways to identify and/or diagnose the presence, severity, or both of a disease or disorder (such as, for example, cancer).

As used herein, “effective amount” and “amount effective” can refer to an amount that is sufficient to achieve the desired result such as, for example, the treatment and/or prevention of a disease or disorder (e.g., a hematological cancer) or a suspected disease or disorder (e.g., a hematological cancer). As used herein, the terms “effective amount” and “amount effective” can refer to an amount that is sufficient to achieve the desired an effect on an undesired condition (e.g., a hematological cancer). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount the disclosed chimeric fusion proteins, the disclosed isolated nucleic acid molecules, the disclosed vectors, the disclosed cells, the disclosed pharmaceutical formulations, the disclosed anti-chemokines, the disclosed anti-cancer agents, the disclosed chemotherapeutics, or any combination thereof that (i) treats the particular disease, condition, or disorder (e.g., a hematological cancer), (ii) attenuates, ameliorates, or eliminates one or more symptoms of the particular disease, condition, or disorder e.g., a hematological cancer), or (iii) delays the onset of one or more symptoms of the particular disease, condition, or disorder described herein (e.g., a hematological cancer). The specific therapeutically effective dose level for any particular patient will depend upon a variety of factors including the disorder being treated and the severity of the disorder; one or more of the disclosed chimeric fusion proteins, the disclosed isolated nucleic acid molecules, the disclosed vectors, the disclosed cells, the disclosed pharmaceutical formulations, or any combination thereof employed; the disclosed methods employed; the age, body weight, general health, sex and diet of the patient; the time of administration; the route of administration; the rate of excretion of one or more of the disclosed chimeric fusion proteins, the disclosed isolated nucleic acid molecules, the disclosed vectors, the disclosed cells, the disclosed pharmaceutical formulations, or any combination thereof employed; the duration of the treatment; drugs used in combination or coincidental with the one or more of the disclosed chimeric fusion proteins, the disclosed isolated nucleic acid molecules, the disclosed vectors, the disclosed cells, the disclosed pharmaceutical formulations, or any combination thereof employed, and other like factors well known in the medical arts. For example, it is well within the skill of the art to start doses of the disclosed chimeric fusion proteins, the disclosed isolated nucleic acid molecules, the disclosed vectors, the disclosed cells, the disclosed pharmaceutical formulations, the disclosed anti-chemokines, the disclosed anti-cancer agents, the disclosed chemotherapeutics, or any combination thereof at levels lower than those required to achieve the desired therapeutic effect and to gradually increase the dosage until the desired effect is achieved. If desired, then the effective daily dose can be divided into multiple doses for purposes of administration. Consequently, a single dose of the disclosed chimeric fusion proteins, the disclosed isolated nucleic acid molecules, the disclosed vectors, the disclosed cells, the disclosed pharmaceutical formulations, the disclosed anti-chemokines, the disclosed anti-cancer agents, the disclosed chemotherapeutics, or any combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, a hematological cancer.

The term “antibody” (Ab) includes, without limitation, a glycoprotein immunoglobulin that binds specifically to an antigen. An antibody can comprise at least two heavy (H) chains and two light (L) chains interconnected by disulfide bonds, or an antigen-binding molecule thereof. Each H chain can comprise a heavy chain variable region (abbreviated herein as VH) and a heavy chain constant region. The heavy chain constant region can comprise three constant domains, CH1, CH2 and CH3. Each light chain can comprise a light chain variable region (abbreviated herein as VL) and a light chain constant region. The light chain constant region can comprise one constant domain, CL. The VH and VL regions can be further subdivided into regions of hypervariability, termed complementarity determining regions (CDRs), interspersed with regions that are more conserved, termed framework regions (FR). Each VH and VL can comprise three CDRs and four FRs, arranged from amino-terminus to carboxy-terminus in the following order: FR1, CDR1, FR2, CDR2, FR3, CDR3, and FR4. The variable regions of the heavy and light chains can contain a binding domain that interacts with an antigen. The constant regions of the Abs can mediate the binding of the immunoglobulin to host tissues or factors, including various cells of the immune system (e.g., effector cells) and the first component (C1q) of the classical complement system. Generally, human antibodies can be approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains can be linked or connected to one another by a single disulfide bond and two other disulfide bonds can connect the heavy chain hinge regions to one another, so that the dimers can be connected to one another and the tetramer can be formed. Naturally produced antibodies are also glycosylated, e.g., on the CH₂ domain. The term “antibody” is used to mean an immunoglobulin molecule that recognizes and specifically binds to a target, such as a protein, polypeptide, peptide, carbohydrate, polynucleotide, lipid, or combinations of the foregoing etc., through at least one antigen recognition site within the variable region of the immunoglobulin molecule. As used herein, the term encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, fusion proteins comprising an antibody portion, and any other modified immunoglobulin molecule comprising an antigen recognition site so long as the antibodies exhibit the desired biological activity. An antibody can be of any the five major classes of immunoglobulins: IgA, IgD. IgE, IgG, and IgM, or subclasses (isotypes) thereof (e.g., IgG1. IgG2, IgG3. IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as toxins, radioisotopes, etc.

The term “variable region” or “variable domain” is used interchangeably. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In an aspect, the variable region can be a human variable region. In an aspect, the variable region comprises rodent or murine CDRs and human framework regions (FRs). In an aspect, the variable region is a primate (e.g., non-human primate) variable region. In an aspect, the variable region comprises rodent or murine CDRs and primate framework regions (FRs).

The terms “VL” and “VL domain” are used interchangeably to refer to the light chain variable region of an antibody or an antigen-binding molecule thereof. The terms “VH” and “VH domain” are used interchangeably to refer to the heavy chain variable region of an antibody or an antigen-binding molecule thereof.

As used herein, “conjugate” or “conjugated” can be used to define the operative association of one disclosed component to another disclosed component. In an aspect, conjugated does not intend to refer solely to any type of operative association and is not particularly limited to chemical “conjugation”.

As known to the skilled person, IgG can bind to cell surface receptors on many types of cells to bring about an assortment of effects, for example, (i) the enabling of phagocytosis (e.g., monocytes, macrophages, neutrophils), (ii) antibody-dependent cellular cytotoxicity (monocytes, macrophages and lymphocytes), or (iii) to effect feedback control on antibody synthesis (B and T lymphocytes). In an aspect, the properties of the IgG subclasses can very, and in most cases the Fc fragments can have the same property as the intact IgG; meaning that it does not appear to be modulated by the hinge or the Fab. In an aspect, cellular Fc receptors can be classified into three categories according to structure and affinity. All the sites on IgG that can interact with these separate receptors appear to be located in the Fc region, and, in the case of the FcγRI (which is the highest affinity receptor class), the site involves residues 233-237, at the N-terminal end of the Cγ2 domain close to the hinge region but coded in the Cγ2 exon.

“Endogenous” with reference to a gene, protein, and/or nucleic acid refers to the natural presence of that gene, protein, and/or nucleic acid in a cell, such as an immune cell.

“Exogenous” refers to an introduced agent, such as a nucleic acid, gene, or protein, into a cell, for example from an outside source. A nucleic acid introduced into a cell is exogenous even if it encodes a protein which is naturally found in the cell. Such exogenous introduction of a nucleic acid encoding a protein can be used to increase the expression of the protein over the level that would naturally be found in the cell under similar conditions, e.g., without introduction of the exogenous nucleic acid.

A “T cell receptor” or “TCR” refers to antigen-recognition molecules present on the surface of T cells. During normal T cell development, each of the four TCR genes, a, 0, y, and 6, may rearrange leading to highly diverse TCR proteins.

As used herein, “effector function” can refer to a biological result of interaction of an antibody Fc region with an Fc receptor or ligand. Effector functions comprise, without limitation, antibody-dependent cell-mediated cytotoxicity (ADCC), antibody-dependent cell-mediated phagocytosis (ADCP), and complement mediated cytotoxicity (CMC). An effector function may be antigen binding dependent, antigen binding independent, or both. ADCC refers to lysis of antibody-bound target cells by immune effector cells. Without wishing to be bound by any theory, ADCC is generally understood to involve Fc receptor (FcR)-bearing effector cells recognizing and subsequently killing antibody-coated target cells (e.g., cells that express on their surface antigens to which an antibody is bound). Effector cells that mediate ADCC may comprise immune cells, comprising yet not limited to, one or more of natural killer (NK) cells, macrophages, neutrophils, eosinophils.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy can include, but are not limited to, NK cells and T cell therapies. T cell therapy can include adoptive T cell therapy, tumor-infiltrating lymphocyte (TIL) immunotherapy, autologous cell therapy, engineered autologous cell therapy (eACTTM), and allogeneic T cell transplantation. However, one of skill in the art would recognize that the conditioning methods disclosed herein would enhance the effectiveness of any transplanted T cell therapy. The T cells or NK cells of the immunotherapy can come from any source known in the art. For example, T cells and NK cells can be differentiated in vitro from a hematopoietic stem cell population (for example iPSCs) or can be obtained from a subject. T cells and NK cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using techniques known to the skilled person.

As used herein, the term “humanized antibody” refers to forms of non-human (e.g., murine) antibodies that are specific immunoglobulin chains, chimeric immunoglobulins, or fragments thereof that contain minimal non-human sequences. Typically, humanized antibodies are human immunoglobulins in which residues from the complementary determining region (CDR) are replaced by residues from the CDR of a non-human species (e.g., mouse, rat, rabbit, hamster, etc.) that have the desired specificity, affinity, and capability. In some instances, the Fv framework region (FR) residues of a human immunoglobulin are replaced with the corresponding residues in an antibody from a non-human species that has the desired specificity, affinity, and capability. The humanized antibody can be further modified by the substitution of additional residue either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin.

That an antibody “selectively binds” or “specifically binds” to an epitope or receptor means that the antibody reacts or associates more frequently, more rapidly, with greater duration, with greater affinity, or with some combination of the above to the epitope or receptor than with alternative substances, including unrelated proteins. “Selectively binds” or “specifically binds” means, for instance, that an antibody binds to a protein with a KD of about 0.1 mM or less, more usually about 1 M or less. “Selectively binds” or “specifically binds” means at times that an antibody binds to a protein with a KD of about 0.1 mM or less, at times about 1 M or less, at times about 0.1 M or less, at times about 0.01 M or less, and at times about 1 nM or less. It is understood that, in an aspect, an antibody or binding moiety that specifically binds to a first target may or may not specifically bind to a second target. As such, “specific binding” does not necessarily require (although it can include) exclusive binding, e.g., binding to a single target.

A “target” or “target antigen” is any molecule bound by a binding motif (e.g., a PS-expressing hematological cancer cell). A disclosed target can be cells and/or tissues in a subject.

“Antigen-specific targeting region” (ASTR) refers to the region of a disclosed chimeric fusion protein that targets specific antigens. In an aspect, the antigen-specific targeting regions comprise an antibody or a functional equivalent thereof or a fragment thereof or a derivative thereof and each of the targeting regions target a different antigen. The targeting regions may comprise full length heavy chain, Fab fragments, single chain Fv (scFv) fragments, divalent single chain antibodies or diabodies, each of which are specific to the target antigen.

The term “autologous” refers to any material derived from the same individual to which it is later to be re-introduced. For example, a subject's own cells can be obtained, made to express one or more disclosed CARs, and then administered to the same subject.

As used herein, “RNA therapeutics” can refer to the use of oligonucleotides to target RNA. RNA therapeutics can offer the promise of uniquely targeting the precise nucleic acids involved in a particular disease with greater specificity, improved potency, and decreased toxicity. This could be particularly powerful for genetic diseases where it is most advantageous to aim for the RNA as opposed to the protein. In an aspect, a therapeutic RNA can comprise one or more expression sequences. As known to the art, expression sequences can comprise an RNAi, shRNA, mRNA, non-coding RNA (ncRNA), an antisense such as an antisense RNA, miRNA, morpholino oligonucleotide, peptide-nucleic acid (PNA) or ssDNA (with natural, and modified nucleotides, including but not limited to, LNA, BNA, 2′-O-Me-RNA, 2′-MEO-RNA, 2′-F-RNA), or analog or conjugate thereof. In an aspect, a disclosed therapeutic RNA can comprise one or more long non-coding RNA (lncRNA), such as, for example, a long intergenic non-coding RNA (lincRNA), pre-transcript, pre-miRNA, pre-mRNA, competing endogenous RNA (ceRNA), small nuclear RNA (snRNA), small nucleolar RNA (snoRNA), pseudo-gene, rRNA, or tRNA. In an aspect, ncRNA can be piwi-interacting RNA (piRNA), primary miRNA (pri-miRNA), or premature miRNA (pre-miRNA). In an aspect, a disclosed therapeutic RNA or an RNA therapeutic can comprise antisense oligonucleotides (ASOs) that inhibit mRNA translation, oligonucleotides that function via RNA interference (RNAi) pathway, RNA molecules that behave like enzymes (ribozymes), RNA oligonucleotides that bind to proteins and other cellular molecules, and ASOs that bind to mRNA and form a structure that is recognized by RNase H resulting in cleavage of the mRNA target. In an aspect, RNA therapeutics can comprise RNAi and ASOs that inhibit mRNA translation. Generally speaking, as known to the art, RNAi operates sequence specifically and post-transcriptionally by activating ribonucleases which, along with other enzymes and complexes, coordinately degrade the RNA after the original RNA target has been cut into smaller pieces while antisense oligonucleotides bind to their target nucleic acid via Watson-Crick base pairing, and inhibit or alter gene expression via steric hindrance, splicing alterations, initiation of target degradation, or other events.

As used herein, the terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

The terms “proliferative disorder” and “proliferative disease” refer to disorders associated with abnormal cell proliferation such as cancer.

“Tumor” and “neoplasm” as used herein refer to any mass of tissue that result from excessive cell growth or proliferation, either benign (noncancerous) or malignant (cancerous) including pre-cancerous lesions.

“Metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

The terms “cancer stem cell” or “tumor stem cell” or “solid tumor stem cell” are used interchangeably herein and refer to a population of cells from a solid tumor that: (1) have extensive proliferative capacity; (2) are capable of asymmetric cell division to generate one or more kinds of differentiated progeny with reduced proliferative or developmental potential; and (3) are capable of symmetric cell divisions for self-renewal or self-maintenance. These properties of “cancer stem cells” or “tumor stem cells” or “solid tumor stem cells” confer on those cancer stem cells the ability to form palpable tumors upon serial transplantation into an immunocompromised mouse compared to the majority of tumor cells that fail to form tumors. Cancer stem cells undergo self-renewal versus differentiation in a chaotic manner to form tumors with abnormal cell types that can change over time as mutations occur.

The terms “cancer cell” or “tumor cell” and grammatical equivalents refer to the total population of cells derived from a tumor including both non-tumorigenic cells, which comprise the bulk of the tumor cell population, and tumorigenic stem cells (cancer stem cells).

As used herein “tumorigenic” refers to the functional features of a solid tumor stem cell including the properties of self-renewal (giving rise to additional tumorigenic cancer stem cells) and proliferation to generate all other tumor cells (giving rise to differentiated and thus non-tumorigenic tumor cells) that allow solid tumor stem cells to form a tumor.

As used herein, the “tumorigenicity” of a tumor refers to the ability of a random sample of cells from the tumor to form palpable tumors upon serial transplantation into immunocompromised mice.

As used herein, “lipid nanoparticles” or “LNPs” can deliver nucleic acid (e.g., DNA or RNA), protein (e.g., RNA-guided DNA binding agent), or nucleic acid together with protein. LNPs can comprise biodegradable, ionizable lipids. For example, LNPs can comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. In an aspect, the term cationic and ionizable in the context of LNP lipids can be used interchangeably, e.g., wherein ionizable lipids are cationic depending on the pH.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of a missing, deficient, and/or mutant protein or enzyme. It should be understood that sequence with substantial sequence identity do not necessarily have the same length and may differ in length. For example, sequences that have the same nucleotide sequence but of which one has additional nucleotides on the 3″- and/or 5″-side are 100% identical.

As used herein, “immune-modulating” refers to the ability of one or more of the disclosed chimeric fusion proteins, the disclosed isolated nucleic acid molecules, the disclosed vectors, the disclosed cells, the disclosed pharmaceutical formulations, or a combination thereof to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including lymphokines and antibodies, that have autocrine, paracrine, and endocrine effects on immune cells.

As used herein, “immune modulator” refers to an agent that is capable of adjusting a given immune response to a desired level (e.g., as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGF1R antibodies, an IL2 mutein, a BTK inhibitor, or a combination thereof. In an aspect, a disclosed immune modulator can comprise one or more Treg (regulatory T cells) infusions (e.g., antigen specific Treg cells to AAV). In an aspect, a disclosed immune modulator can be bortezomib or SVP-Rapamycin. In an aspect, an immune modulator can be administered by any suitable route of administration including, but not limited to, in utero, intra-CSF, intrathecally, intravenously, subcutaneously, transdermally, intradermally, intramuscularly, orally, transcutaneously, intraperitoneally (IP), or intravaginally. In an aspect, a disclosed immune modulator can be administered using a combination of routes. Administration can also include hepatic intra-arterial administration or administration through the hepatic portal vein (HPV). Administration of an immune modulator can be continuous or intermittent, and administration can comprise a combination of one or more routes.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., one or more of the disclosed chimeric fusion proteins, the disclosed isolated nucleic acid molecules, the disclosed vectors, the disclosed cells, the disclosed pharmaceutical formulations, or a combination thereof) can be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy to a subject having or diagnosed with hematological cancer.

Disclosed are the components to be used to prepare the disclosed isolated nucleic acid molecules, disclosed vectors, or disclosed pharmaceutical formulations as well as the disclosed isolated nucleic acid molecules, disclosed vectors, or disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific claimed compositions and/or claimed methods.

### B. Hematological Cancers

Hematologic cancers begin in the cells of the immune system or in blood-forming tissue, such as the bone marrow. Common types of hematologic cancer include lymphoma, myeloma, and leukemia. Myeloid neoplasms include acute myeloid leukemia (AML) and related neoplasms, myeloproliferative neoplasms (Ph negative) and chronic myeloid leukemia, myelodysplastic syndromes (MDS), and myeloproliferative/myelodysplastic syndromes.

Acute myeloid leukemia (AML) and related neoplasms include AML with recurrent genetic abnormalities, AML with myelodysplasia-related changes, therapy-related AML, AML, not otherwise specified, and acute leukemias of ambiguous lineage.

Myeloproliferative neoplasms (Ph negative) and chronic myeloid leukemia include essential thrombocythemia, polycythemia vera, primary myelofibrosis, systemic mastocytosis, chronic myeloid leukemia, chronic neutrophilic leukemia, and chronic eosinophilic leukemia. Myelodysplastic syndromes (MDS) include refractory anemia with ringed sideroblasts, refractory cytopenia with multilineage dysplasia, refractory anemia with excess blasts (type I), refractory anemia with excess blasts (type 2), MDS with isolated del (5q), and MDS (unclassifiable). Myeloproliferative/myelodysplatic include chronic myelomonocytic leukemia, atypical chronic myeloid leukemia, juvenile myelomonocytic leukemia, myeloproliferative/myelodysplatic syndromes (unclassifiable)).

Lymphoid neoplasms include precursor lymphoid neoplasms, mature B-cell neoplasms, and Hodgkin lymphoma. Precursor lymphoid neoplasms include B lymphoblastic leukemia/lymphoma and T lymphoblastic leukemia/lymphoma.

Mature B-cell neoplasms include diffuse large B-cell lymphoma, primary central nervous system lymphoma, primary mediastinal B-cell lymphoma, Burkitt lymphoma/leukemia Follicular lymphoma, chronic lymphocytic leukemia/small lymphocytic lymphoma, B-cell prolymphocytic leukemia, lymphoplasmacytic lymphoma/Waldenstrom macroglobulinemia, mantle cell lymphoma, marginal zone lymphomas, post-transplant lymphoproliferative disorders, HIV-associated lymphomas, primary effusion lymphoma, intravascular large B-cell lymphoma, primary cutaneous B-cell lymphoma, and hairy cell leukemia.

Mature B-cell neoplasms include multiple myeloma and multiple myeloma includes monoclonal gammopathy of unknown significance, smoldering multiple myeloma, and solitary plasmacytomas (solitary bone and extramedullary). As shown below, there are several markers for hematologic malignancies.

| Name | Normal Expression | Comments |
|---|---|---|
| CD1a | Immature T cells; Langerhans | Associated with ALL |
| | cells T cells and NK cells | |
| CD2 | T cells | May be aberrantly expressed in AML and |
| | | systemic mastocytosis |
| CD3 | T-cell subset, monocytes, | Indicates T-cell lineage |
| | histiocytes | |
| CD4 | T cells and B-cell subset | Associated with mature T-cell lymphoid |
| | | neoplasms and monocytic AML |
| CD5 | T cells and NK cells | Indicates T-cell lineage; may be expressed |
| | | aberrantly on B cells, such as CLL and |
| | | MCL |
| CD7 | Immature T cells; Langerhans | Indicator of T-cell lineage; may be |
| | cells T cells and NK cells | aberrantly expressed in AML, MDS, and |
| | | MPN |
| CD8 | T-cell subset and some NK cells | Associated with some mature T-cell |
| | | lymphoid neoplasms Associated with |
| | | precursor B-cell ALL |
| CD9 | Precursor B cells, activated T | |
| | cells, and platelets | |
| CD10 | Immature T cells and B cells, | Associated with ALL, some mature T-cell |
| | subset of mature T cells and B | neoplasms, and some mature B-cell |
| | cells, and neutrophils | neoplasms. In mature B-cell neoplasms, it |
| | | is associated with a germinal center |
| | | phenotype of FL and DLBCL. |
| CD11b | Maturing neutrophilic and | Aberrantly expressed in AML, MDS, and |
| | monocytic cells and some | MPN |
| | lymphoid cells | |
| CD11c | Subset of B cells and subset of T | Associated with HCL; occasionally with |
| | cells Myeloid and monocytic cells | CLL and MCL |
| CD13 | | Indicates neutrophilic and monocytic |
| | | lineage. Associated with myeloid |
| | | neoplasms; may be aberrantly expressed in |
| | | B-cell neoplasms, MDS, and MPN. |
| CD14 | Monocytes | Associated with monocytic AML |
| CD15 | Myeloid and monocytic cells | May be aberrantly expressed in AML, |
| | | MDS, and MPD |
| CD16 | NK cells, NK/T cells, monocytes, | Indicates NK differentiation; may be |
| | and maturing neutrophilic cells | aberrantly expressed in AML, MDS, and |
| | | MPN |
| CD19 | All B cells including | Indicates B-cell lineage |
| | lymphoblasts, mature B cells, and | |
| | most plasma cells | |
| CD20 | Mature B cells (except plasma | Indicates B-cell lineage; aberrant |
| | cells) and small subset of T cells | expression in ALL |
| CD22 | Cytoplasmic expression in early | Indicates B-cell lineage in ALL and |
| | B cells and surface expression | mature B-cell neoplasms |
| | acquired during maturation of | |
| | precursor B cells | |
| CD23 | Weak expression on resting B | Distinguishes between CD5⁺ mature B- |
| | cells; intensity increases with | lymphoid neo-plasms (e.g., CLL is |
| | activation | CD5⁺/CD23⁺ and MCL is CD5⁺/CD23⁻) |
| CD25 | Activated B cells and activated T | Associated with ATLL and HCL; variable |
| | cells | expression in other mature T-cell |
| | | neoplasms and systemic mastocytosis |
| CD26 | Immature T cells, NK cells, and | Associated with CTCL/Sézary syndrome |
| | activated T cells | |
| CD30 | Activated T cells and B cells, and | Associated with HL and ALCL |
| | monocytes | |
| CD33 | Myeloid and monocytic cells | |
| CD34 | Hematopoietic precursors, B-cell | Associated with AML and ALL blasts and |
| | and T- cell precursors and | one of the stem cell markers |
| | myeloblasts | |
| CD36 | Monocytes, erythroid cells, | When used in combination with CD64, is a |
| | megakaryocytes, and platelets | more sensitive marker of monocytic |
| | | differentiation than CD14 |
| CD38 | Precursor B cells (hematogones), | Associated with plasmacytic |
| | normal follicle center B cells, | differentiation and multiple myeloma; |
| | immature and activated T cells, | poor prognostic marker in CLL/SLL |
| | plasma cells (bright intensity), | |
| | myeloid and monocytic cells, and | |
| | erythroid precursors | |
| CD41 | Megakaryocytes and platelets | Associated with megakaryocytic |
| | | differentiation Aberrant expression in |
| | | CLL, MCL, and some MZL |
| CD43 | T cells, myeloid, monocytes, and | Associated with megakaryocytic |
| | small B-cell subset | differentiation Aberrant expression in |
| | | CLL, MCL, and some MZL |
| CD45 | All B cells and T cells; weaker | |
| | intensity on precursors and | |
| | plasma cells | |
| CD45RA | B-cell subsets, T-cell subsets, | Useful to distinguish between precursor |
| | including mostly naïve T cells | lymphoid neoplasms from mature |
| | | lymphoid neoplasms; identifies blasts in |
| | | acute leukemia |
| CD45RO | B-cell subsets, T-cell subsets, | — |
| | including mostly memory T cells | |
| CD52 | Thymocytes, T and B cells (not | — |
| | plasma cells), monocytes, | |
| | macrophages | |
| CD56 | NK cells and NK/T cells | — |
| CD58 | Leukocytes including bright- | Indicates NK differentiation; aberrant |
| | intensity staining of precursors; | expression in AML, MM, MDS, and MPN |
| | intensity decreases with | |
| | maturation | |
| CD61 | Megakaryocytes and platelets | Distinguishes ALL from normal precursor |
| | | B-cell (hematogones) including detection |
| | | of MRD |
| CD64 | Monocytes and intermediate | Associated with megakaryocytic |
| | neutrophilic precursors | differentiation |
| CD71 | Erythroid precursors, myeloid, | Identification of immature erythroid cells; |
| | activated lymphoid, and | possibly ex-pressed in MDS |
| | proliferating cells | |
| CD79a | Precursor B cells, plasma cells | Indicates B-cell lineage in ALL and |
| and | positive, variable expression | mature lymphoid neoplasms; intensity |
| CD79b | mature B cells | often differs between subtypes of mature - |
| | | cell neoplasm; may be seen in some T-cell |
| | | lymphoid neoplasms |
| CD103 | B-cell subset and intramucosal T | Associated with HCL, EATL, and some |
| | cells | MZL |
| CD117 | Immature myeloid cells and mast | Associated with myeloblasts; may be |
| | cells | aberrantly expressed MM and MGUS |
| CD123 | Monocytes, neutrophils, basophils | May be expressed with monocytic AML |
| | megakaryocytes, and | |
| | plasmacytoid dendritic cells. | |
| CD138 | Plasma cells | |
| CD163 | Monocytes and macrophages | Indicates monocytic differentiation |
| FMC-7 | B cells | Distinguishes CD5⁺ lymphoid neoplasm: |
| | | CLL is FMC-7 negative, and MCL is |
| | | FMC-7 positive. Associated with HCL. |
| Bcl-2 | T cells and some B cells; absent | Distinguishes CD10⁺ lymphoid neoplasms: |
| | on nor mal germinal center cells | FL is usually positive, BL negative, |
| | | variable in DLBCL. |
| HLA-DR | Myeloblasts, monocytes, | Associated with APL; may be aberrantly |
| | promyelocytes all B cells, and | expressed in AML, MDS, and MPN |
| | activated T cells | |
| Kappa | Mature B cells | Immunoglobulin light chain restriction in |
| and | | B cells |
| lambda | | |
| MPO | Neutrophilic and monocytic cells | Indicates myeloid differentiation; in |
| | | contrast to cytochemical stain, measures |
| | | the presence of antigen, not enzyme |
| | | activity |
| TCR | T-cell receptor on mature T cells | Used in classification of mature T-cell |
| | | neoplasms |
| TdT | B-cell and T-cell precursors | Associated with ALL and some AML |
| ZAP-70 | T cells, NK cells, precursor B | Poor prognostic marker in CLL/SLL |
| | cells | |

### C. Annexins

ANXA1 (Annexin A1) is a protein-coding gene. This gene encodes a membrane-localized protein that binds phospholipids. This protein inhibits phospholipase A2 and has anti-inflammatory activity. Loss of function or expression of this gene has been detected in multiple tumors. Diseases associated with ANXA1 include Shoulder Impingement Syndrome and Brain Edema. Among its related pathways are GPCR downstream signaling and Class A/1 (Rhodopsin-like receptors). Gene Ontology (GO) annotations related to this gene include calcium ion binding and signaling receptor binding. An important paralog of this gene is ANXA2. ANXA1 plays important roles in the innate immune response as effector of glucocorticoid-mediated responses and regulator of the inflammatory process. ANXA1 has anti-inflammatory activity. ANXA1 plays a role in glucocorticoid-mediated down-regulation of the early phase of the inflammatory response (by similarity). ANXA1 contributes to the adaptive immune response by enhancing signaling cascades that are triggered by T-cell activation, regulates differentiation and proliferation of activated T-cells. ANXA1 promotes the differentiation of T-cells into Th1 cells and negatively regulates differentiation into Th2 cells. ANXA1 has no effect on unstimulated T cells. ANXA1 negatively regulates hormone exocytosis via activation of the formyl peptide receptors and reorganization of the actin cytoskeleton. ANXA1 has high affinity for Ca(2+) and can bind up to eight Ca(2+) ions (by similarity). ANXA1 displays Ca(2+)-dependent binding to phospholipid membranes. ANXA1 plays a role in the formation of phagocytic cups and phagosomes and plays a role in phagocytosis by mediating the Ca(2+)-dependent interaction between phagosomes and the actin cytoskeleton (by similarity). ANXA1 functions at least in part by activating the formyl peptide receptors and downstream signaling cascades. ANXA1 promotes chemotaxis of granulocytes and monocytes via activation of the formyl peptide receptors and promotes rearrangement of the actin cytoskeleton, cell polarization and cell migration. ANXA1 promotes resolution of inflammation and wound healing. ANXA1 acts via neutrophil N-formyl peptide receptors to enhance the release of CXCL2.

ANXA2 (Annexin A2) is a protein coding gene. This gene encodes a member of the annexin family. Members of this calcium-dependent phospholipid-binding protein family play a role in the regulation of cellular growth and in signal transduction pathways. This protein functions as an autocrine factor which heightens osteoclast formation and bone resorption. This gene has three pseudogenes located on chromosomes 4, 9 and 10, respectively. Multiple alternatively spliced transcript variants encoding different isoforms have been found for this gene. Annexin A2 expression has been found to correlate with resistance to treatment against various cancer forms. Diseases associated with ANXA2 include Antiphospholipid Syndrome and Acute Promyelocytic Leukemia. Among its related pathways are Interleukin-12 family signaling and Innate Immune System. Gene Ontology (GO) annotations related to this gene include RNA binding and small GTPase binding. An important paralog of this gene is ANXA1. Calcium-regulated membrane-binding protein whose affinity for calcium is greatly enhanced by anionic phospholipids. It binds two calcium ions with high affinity. May be involved in heat-stress response. Inhibits PCSK9-enhanced LDLR degradation, probably reduces PCSK9 protein levels via a translational mechanism but also competes with LDLR for binding with PCSK9. ANXA2 binds M. pneumoniae CARDS toxin, probably serves as one receptor for this pathogen. When ANXA2 is down-regulated by siRNA, less toxin binds to human cells and less vacuolization (a symptom of M. pneumoniae infection) is seen.

ANXA3 (Annexin A3) is a protein coding gene. This gene encodes a member of the annexin family. Members of this calcium-dependent phospholipid-binding protein family play a role in the regulation of cellular growth and in signal transduction pathways. This protein functions in the inhibition of phospholipase A2 and cleavage of inositol 1,2-cyclic phosphate to form inositol 1-phosphate. This protein may also play a role in anti-coagulation. Diseases associated with ANXA3 include ovarian cancer and prostate cancer. Among its related pathways are prostaglandin synthesis and regulation. Gene Ontology (GO) annotations related to this gene include calcium ion binding and calcium-dependent phospholipid binding. An important paralog of this gene is ANXA11. Inhibitor of phospholipase A2, also possesses anti-coagulant properties. Also cleaves the cyclic bond of inositol 1,2-cyclic phosphate to form inositol 1-phosphate.

ANXA4 (Annexin A4) is a protein coding gene. Annexin IV (ANX4) belongs to the annexin family of calcium-dependent phospholipid binding proteins. Although their functions are still not clearly defined, several members of the annexin family have been implicated in membrane-related events along exocytotic and endocytotic pathways. ANX4 has 45 to 59% identity with other members of its family and shares a similar size and exon-intron organization. Isolated from human placenta, ANX4 encodes a protein that has possible interactions with ATP and has in vitro anticoagulant activity and also inhibits phospholipase A2 activity. ANX4 is almost exclusively expressed in epithelial cells. Several transcript variants encoding different isoforms have been found for this gene. Among its related pathways are prostaglandin synthesis and regulation. Gene Ontology (GO) annotations related to this gene include calcium ion binding and calcium-dependent protein binding. An important paralog of this gene is ANXA11. Calcium/phospholipid-binding protein which promotes membrane fusion and is involved in exocytosis.

ANXA5 (Annexin A5) is a protein coding gene. The Annexin 5 gene spans 29 kb containing 13 exons, and encodes a single transcript of approximately 1.6 kb and a protein product with a molecular weight of about 35 kDa. The protein encoded by this gene belongs to the annexin family of calcium-dependent phospholipid binding proteins some of which have been implicated in membrane-related events along exocytotic and endocytotic pathways. Annexin 5 is a phospholipase A2 and protein kinase C inhibitory protein with calcium channel activity and a potential role in cellular signal transduction, inflammation, growth and differentiation. Annexin 5 has also been described as placental anticoagulant protein I, vascular anticoagulant-alpha, endonexin II, lipocortin V, placental protein 4 and anchorin CII. Polymorphisms in this gene have been implicated in various obstetric complications. Diseases associated with ANXA5 include Pregnancy Loss, Recurrent 3 and Antiphospholipid Syndrome. Among its related pathways are Response to elevated platelet cytosolic Ca2+ and Regulation of CFTR activity (norm and CF). Gene Ontology (GO) annotations related to this gene include calcium ion binding and calcium-dependent phospholipid binding. An important paralog of this gene is ANXA4. This protein is an anticoagulant protein that acts as an indirect inhibitor of the thromboplastin-specific complex, which is involved in the blood coagulation cascade.

ANXA6 (Annexin A6) is a protein coding gene. Annexin VI belongs to a family of calcium-dependent membrane and phospholipid binding proteins. Several members of the annexin family have been implicated in membrane-related events along exocytotic and endocytotic pathways. The annexin VI gene is approximately 60 kbp long and contains 26 exons. It encodes a protein of about 68 kDa that consists of eight 68-amino acid repeats separated by linking sequences of variable lengths. It is highly similar to human annexins I and II sequences, each of which contain four such repeats. Annexin VI has been implicated in mediating the endosome aggregation and vesicle fusion in secreting epithelia during exocytosis. Alternatively spliced transcript variants have been described. Diseases associated with ANXA6 include Kwashiorkor and Malignant Hyperthermia. Among its related pathways are Cardiac conduction and Myometrial relaxation and contraction pathways. Gene Ontology (GO) annotations related to this gene include calcium ion binding and GTP binding. An important paralog of this gene is ANXA11.

ANXA7 (Annexin A7) is a protein coding gene. Annexin VII is a member of the annexin family of calcium-dependent phospholipid binding proteins. The Annexin VII gene contains 14 exons and spans approximately 34 kb of DNA. An alternatively spliced cassette exon results in two mRNA transcripts of 2.0 and 2.4 kb which are predicted to generate two protein isoforms differing in their N-terminal domain. The alternative splicing event is tissue specific and the mRNA containing the cassette exon is prevalent in brain, heart and skeletal muscle. The transcripts also differ in their 3′-non coding regions by the use of two alternative poly(A) signals. Annexin VII encodes a protein with a molecular weight of approximately 51 kDa with a unique, highly hydrophobic N-terminal domain of 167 amino acids and a conserved C-terminal region of 299 amino acids. The latter domain is composed of alternating hydrophobic and hydrophilic segments. Structural analysis of the protein suggests that Annexin VII is a membrane binding protein with diverse properties, including voltage-sensitive calcium channel activity, ion selectivity and membrane fusion. Among its related pathways are Cytoskeletal Signaling and Ca, cAMP and Lipid Signaling. Gene Ontology (GO) annotations related to this gene include RNA binding and integrin binding. An important paralog of this gene is ANXA11.

ANXA8 (Annexin A8) is a protein-coding gene. This gene encodes a member of the annexin family of evolutionarily conserved Ca2+ and phospholipid-binding proteins. The encoded protein may function as an anticoagulant that indirectly inhibits the thromboplastin-specific complex. Overexpression of this gene has been associated with acute myelocytic leukemia. A highly similar duplicated copy of this gene is found in close proximity on the long arm of chromosome 10. Diseases associated with ANXA8 include Breast Adenocarcinoma and Heterophyiasis. Gene Ontology (GO) annotations related to this gene include calcium ion binding and calcium-dependent phospholipid binding. An important paralog of this gene is ANXA8L1. This protein is an anticoagulant protein that acts as an indirect inhibitor of the thromboplastin-specific complex, which is involved in the blood coagulation cascade.

ANXA8L1 (Annexin A8 Like 1) is a protein-coding gene. Among its related pathways are Prostaglandin synthesis and regulation. Gene Ontology (GO) annotations related to this gene include calcium ion binding and calcium-dependent phospholipid binding. An important paralog of this gene is ANXA8. This gene encodes a member of the annexin family of evolutionarily conserved Ca2+ and phospholipid-binding proteins. The encoded protein may function as an anticoagulant that indirectly inhibits the thromboplastin-specific complex. Overexpression of this gene has been associated with acute myelocytic leukemia. A highly similar duplicated copy of this gene is found in close proximity on the long arm of chromosome 10.

ANXA9 (Annexin A9) is a protein coding gene. The annexins are a family of calcium-dependent phospholipid-binding proteins. Members of the annexin family contain 4 internal repeat domains, each of which includes a type II calcium-binding site. The calcium-binding sites are required for annexins to aggregate and cooperatively bind anionic phospholipids and extracellular matrix proteins. This gene encodes a divergent member of the annexin protein family in which all four homologous type II calcium-binding sites in the conserved tetrad core contain amino acid substitutions that ablate their function. However, structural analysis suggests that the conserved putative ion channel formed by the tetrad core is intact. Diseases associated with ANXA9 include Pemphigus and Acantholytic Acanthoma. Gene Ontology (GO) annotations related to this gene include calcium ion binding and phospholipid binding. An important paralog of this gene is ANXA2. Low affinity receptor for acetylcholine known to be targeted by disease-causing pemphigus vulgaris antibodies in keratinocytes.

ANXA10 (Annexin A10) is a protein coding gene. This gene encodes a member of the annexin family. Members of this calcium-dependent phospholipid-binding protein family play a role in the regulation of cellular growth and in signal transduction pathways. The function of this gene has not yet been determined. Diseases associated with ANXA10 include Hepatocellular Carcinoma. Gene Ontology (GO) annotations related to this gene include calcium ion binding and calcium-dependent phospholipid binding. An important paralog of this gene is ANXA4.

ANXA11 (Annexin A11) is a protein coding gene. This gene encodes a member of the annexin family, a group of calcium-dependent phospholipid-binding proteins. Annexins have unique N-terminal domains and conserved C-terminal domains, which contain calcium-dependent phospholipid-binding sites. The encoded protein is a 56-kD antigen recognized by sera from patients with various autoimmune diseases. Several transcript variants encoding two different isoforms have been identified. Diseases associated with ANXA11 include Amyotrophic Lateral Sclerosis 23 and Inclusion Body Myopathy 0nd Brain White Matter Abnormalities. Gene Ontology (GO) annotations related to this gene include RNA binding and calcium-dependent protein binding. An important paralog of this gene is ANXA7. Binds specifically to calcyclin in a calcium-dependent manner (By similarity). Required for midbody formation and completion of the terminal phase of cytokinesis.

ANXA13 (Annexin A13) is a protein coding gene. This gene encodes a member of the annexin family. Members of this calcium-dependent phospholipid-binding protein family play a role in the regulation of cellular growth and in signal transduction pathways. The specific function of this gene has not yet been determined; however, it is associated with the plasma membrane of undifferentiated, proliferating endothelial cells and differentiated villus enterocytes. Alternatively spliced transcript variants encoding different isoforms have been identified. Gene Ontology (GO) annotations related to this gene include calcium ion binding and phosphatidylserine binding. An important paralog of this gene is ANXA8.

### D. Compositions for Use in the Disclosed Methods

### 1. Chimeric Fusion Proteins

Disclosed herein are chimeric fusion proteins. Disclosed herein are chimeric fusion proteins comprising a phosphatidylserine (PS) binding domain. Disclosed herein is a chimeric fusion protein comprising a phosphatidylserine (PS) binding domain and an immunostimulatory domain. Disclosed herein is a chimeric fusion protein comprising a phosphatidylserine (PS) binding domain operably linked an immunostimulatory domain.

Disclosed herein is a chimeric fusion protein comprising a phosphatidylserine (PS) binding domain, a linker, and an immunostimulatory domain. Disclosed herein is a chimeric fusion protein comprising a phosphatidylserine (PS) binding domain operably linked to an immunostimulatory domain, wherein the immunostimulatory domain further comprises a conjugated small molecule. Disclosed herein are chimeric fusion proteins comprising a secretion signal and a phosphatidylserine (PS) binding domain. Disclosed herein is a chimeric fusion protein comprising a secretion signal, a phosphatidylserine (PS) binding domain, and an immunostimulatory domain. Disclosed herein is a chimeric fusion protein comprising a secretion signal and a phosphatidylserine (PS) binding domain operably linked an immunostimulatory domain. Disclosed herein is a chimeric fusion protein comprising a secretion signal, a phosphatidylserine (PS) binding domain, a linker, and an immunostimulatory domain.

Disclosed herein is a chimeric fusion protein comprising a secretion signal and a phosphatidylserine (PS) binding domain operably linked to an immunostimulatory domain, wherein the immunostimulatory domain further comprise a conjugated small molecule. Disclosed herein is a chimeric fusion protein comprising a PS-binding domain operably linked to an IgG1 Fc domain. Disclosed herein is a chimeric fusion protein comprising a PS-binding domain operably linked to an IgG1 Fc domain and an additional immunostimulatory domain. Disclosed herein is a chimeric fusion protein comprising a PS-binding domain operably linked to an IgG1 Fc domain and an additional immunostimulatory domain, wherein the additional immunostimulatory domain comprises the IgV domain of SIRPα or the extracellular domain of NKG2D protein. Disclosed herein is a chimeric fusion protein comprising a PS-binding domain operably linked to a single-chain antibody (scFV) to a human CD3 protein. Disclosed herein is a chimeric fusion protein comprising an Annexin-binding domain operably linked to a single-chain antibody (scFV) to a human CD3 protein. Disclosed herein is a chimeric fusion protein comprising a PS-binding domain operably linked to a single-chain antibody (scFV) to a human CD3 protein and one or more additional immunostimulatory domains. Disclosed herein is a chimeric fusion protein comprising an Annexin-binding domain operably linked to a single-chain antibody (scFV) to a human CD3 protein and one or more additional immunostimulatory domains.

Disclosed herein is a chimeric fusion protein comprising the sequence set forth in SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:99, SEQ ID NO:100, SEQ ID NO:101, SEQ ID NO:106, or a fragment thereof. In an aspect, a disclosed secretion signal can be any secretion signal known to the art. In an aspect, a disclosed secretion signal can comprise the sequence set forth in SEQ ID NO:107-SEQ ID NO:108.

In an aspect, a disclosed chimeric fusion protein can be humanized or one or more humanized components.

In an aspect of a disclosed chimeric fusion protein, a disclosed phosphatidylserine (PS) binding domain can comprise Annexin A1 (ANXA1) or the PS-binding core domain, Annexin A2 (ANXA1), Annexin A3 (ANXA1), Annexin A4 (ANXA1), Annexin A5 (ANXA1), Annexin A6 (ANXA1), Annexin A7 (ANXA1), Annexin A8 (ANXA1), Annexin A8 Like 1 (ANXA1), Annexin A9 (ANXA1), Annexin A10 (ANXA1), Annexin A11 (ANXA1), Annexin A13 (ANXA1), Adhesion G Protein Coupled Receptor B1 (ADGRB1) or the extracellular domain thereof, Apolipoprotein H (APO-H), Coagulation Factor II (F2), Coagulation Factor VII (F7), Coagulation Factor IX (F9), Coagulation Factor X (F10), Growth Arrest Specific 6 (CAS6), Milk Fat Globule EGF And Factor V/VIII Domain Containing (MFGE8), Advanced Glycosylation End-Product Specific Receptor (AGER) or the extracellular domain thereof, Jumonji Domain-Containing Protein 6 (JMJD6); Protein S (PROS1), Hepatitis A Virus Cellular Receptor 1 (HAVCR1) or the extracellular domain thereof, Hepatitis A Virus Cellular Receptor 2 (HAVCR2) or the extracellular domain thereof, T Cell Immunoglobulin and Mucin Domain Containing (TIM-3) or the extracellular domain thereof, Protein Kinase C Alpha (PRKCA) or the C2 domain thereof, Synaptotagmin (SYT1) or the C2A domain thereof, Stabilin-1 (STAB1) or the extracellular domain thereof, Stabilin-2 (STAB2) or the extracellular domain thereof, or any combination thereof.

In an aspect, a disclosed ANXA1 can comprise the amino acid sequence set forth in SEQ ID NO:01 or a fragment thereof. In an aspect, a disclosed ANXA1 can comprise a sequence having at least 70%, at least 75%, at least 80%, at least 85%, at least 90%, or at least 95% identity to the sequence set forth in SEQ ID NO:01 or a fragment thereof. In an aspect, a disclosed ANXA1 can comprise the amino acid sequence set forth in SEQ ID NO:02 or a fragment thereof. In an aspect, a disclosed ANXA1 can comprise a sequence having at least 70%, at least 75%, at least 80%, at least 85%, at least 90%, or at least 95% identity to the sequence set forth in SEQ ID NO:02 or a fragment thereof. In an aspect, a disclosed ANXA2 can comprise the amino acid sequence set forth in SEQ ID NO:03 or a fragment thereof. In an aspect, a disclosed ANXA2 can comprise a sequence having at least 70%, at least 75%, at least 80%, at least 85%, at least 90%, or at least 95% identity to the sequence set forth in SEQ ID NO:03 or a fragment thereof. In an aspect, a disclosed ANXA3 can comprise the amino acid sequence set forth in SEQ ID NO:04 or a fragment thereof. In an aspect, a disclosed ANXA3 can comprise a sequence having at least 70%, at least 75%, at least 80%, at least 85%, at least 90%, or at least 95% identity to the sequence set forth in SEQ ID NO:04 or a fragment thereof. In an aspect, a disclosed ANXA4 can comprise the amino acid sequence set forth in SEQ ID NO:05 or a fragment thereof. In an aspect, a disclosed ANXA4 can comprise a sequence having at least 70%, at least 75%, at least 80%, at least 85%, at least 90%, or at least 95% identity to the sequence set forth in SEQ ID NO:05 or a fragment thereof. In an aspect, a disclosed ANXA5 can comprise the amino acid sequence set forth in SEQ ID NO:06 or a fragment thereof. In an aspect, a disclosed ANXA5 can comprise a sequence having at least 70%, at least 75%, at least 80%, at least 85%, at least 90%, or at least 95% identity to the sequence set forth in SEQ ID NO:06 or a fragment thereof. In an aspect, a disclosed ANXA6 can comprise the amino acid sequence set forth in SEQ ID NO:07 or a fragment thereof. In an aspect, a disclosed ANXA6 can comprise a sequence having at least 70%, at least 75%, at least 80%, at least 85%, at least 90%, or at least 95% identity to the sequence set forth in SEQ ID NO:07 or a fragment thereof. In an aspect, a disclosed ANXA7 can comprise the amino acid sequence set forth in SEQ ID NO:08 or a fragment thereof. In an aspect, a disclosed ANXA7 can comprise a sequence having at least 70%, at least 75%, at least 80%, at least 85%, at least 90%, or at least 95% identity to the sequence set forth in SEQ ID NO:08 or a fragment thereof. In an aspect, a disclosed ANXA8 can comprise the amino acid sequence set forth in SEQ ID NO:09 or a fragment thereof. In an aspect, a disclosed ANXA8 can comprise a sequence having at least 70%, at least 75%, at least 80%, at least 85%, at least 90%, or at least 95% identity to the sequence set forth in SEQ ID NO:09 or a fragment thereof. In an aspect, a disclosed ANXA8L1 can comprise the amino acid sequence set forth in SEQ ID NO:10 or a fragment thereof. In an aspect, a disclosed ANXA8L1 can comprise a sequence having at least 70%, at least 75%, at least 80%, at least 85%, at least 90%, or at least 95% identity to the sequence set forth in SEQ ID NO:10 or a fragment thereof. In an aspect, a disclosed ANXA9 can comprise the amino acid sequence set forth in SEQ ID NO:11 or a fragment thereof. In an aspect, a disclosed ANXA9 signal peptide can comprise a sequence having at least 70%, at least 75%, at least 80%, at least 85%, at least 90%, or at least 95% identity to the sequence set forth in SEQ ID NO:11 or a fragment thereof. In an aspect, a disclosed ANXA10 can comprise the amino acid sequence set forth in SEQ ID NO:12 or a fragment thereof. In an aspect, a disclosed ANXA10 can comprise a sequence having at least 70%, at least 75%, at least 80%, at least 85%, at least 90%, or at least 95% identity to the sequence set forth in SEQ ID NO:12 or a fragment thereof. In an aspect, a disclosed ANXA11 can comprise the amino acid sequence set forth in SEQ ID NO:13 or a fragment thereof. In an aspect, a disclosed ANXA11 can comprise a sequence having at least 70%, at least 75%, at least 80%, at least 85%, at least 90%, or at least 95% identity to the sequence set forth in SEQ ID NO:13 or a fragment thereof. In an aspect, a disclosed ANXA13 can comprise the amino acid sequence set forth in SEQ ID NO:14 or a fragment thereof. In an aspect, a disclosed ANXA13 can comprise a sequence having at least 70%, at least 75%, at least 80%, at least 85%, at least 90%, or at least 95% identity to the sequence set forth in SEQ ID NO:14 or a fragment thereof.

In an aspect, a disclosed ADGRB1 can comprise the amino acid sequence set forth in SEQ ID NO:15 or a fragment thereof. In an aspect, a disclosed ADGRB1 can comprise a sequence having at least 70%, at least 75%, at least 80%, at least 85%, at least 90%, or at least 95% identity to the sequence set forth in SEQ ID NO:15 or a fragment thereof. In an aspect, a disclosed ADGRB1 can comprise the amino acid sequence set forth in SEQ ID NO:16 or a fragment thereof. In an aspect, a disclosed ADGRB1 can comprise a sequence having at least 70%, at least 75%, at least 80%, at least 85%, at least 90%, or at least 95% identity to the sequence set forth in SEQ ID NO:16 or a fragment thereof. In an aspect, a disclosed APO-H can comprise the amino acid sequence set forth in SEQ ID NO:17 or a fragment thereof. In an aspect, a disclosed APO-H can comprise a sequence having at least 70%, at least 75%, at least 80%, at least 85%, at least 90%, or at least 95% identity to the sequence set forth in SEQ ID NO:17 or a fragment thereof. In an aspect, a disclosed F2 can comprise the amino acid sequence set forth in SEQ ID NO:18 or a fragment thereof. In an aspect, a disclosed F2 can comprise a sequence having at least 70%, at least 75%, at least 80%, at least 85%, at least 90%, or at least 95% identity to the sequence set forth in SEQ ID NO:18 or a fragment thereof. In an aspect, a disclosed F7 can comprise the amino acid sequence set forth in SEQ ID NO:19 or a fragment thereof. In an aspect, a disclosed F7 can comprise a sequence having at least 70%, at least 75%, at least 80%, at least 85%, at least 90%, or at least 95% identity to the sequence set forth in SEQ ID NO:19 or a fragment thereof. In an aspect, a disclosed F9 can comprise the amino acid sequence set forth in SEQ ID NO:20 or a fragment thereof. In an aspect, a disclosed F9 can comprise a sequence having at least 70%, at least 75%, at least 80%, at least 85%, at least 90%, or at least 95% identity to the sequence set forth in SEQ ID NO:20 or a fragment thereof. In an aspect, a disclosed F10 can comprise the amino acid sequence set forth in SEQ ID NO:21 or a fragment thereof. In an aspect, a disclosed F10 can comprise a sequence having at least 70%, at least 75%, at least 80%, at least 85%, at least 90%, or at least 95% identity to the sequence set forth in SEQ ID NO:21 or a fragment thereof. In an aspect, a disclosed GAS6 can comprise the amino acid sequence set forth in SEQ ID NO:22 or a fragment thereof. In an aspect, a disclosed GAS6 an comprise a sequence having at least 70%, at least 75%, at least 80%, at least 85%, at least 90%, or at least 95% identity to the sequence set forth in SEQ ID NO:22 or a fragment thereof. In an aspect, a disclosed MFGE8 can comprise the amino acid sequence set forth in SEQ ID NO:23 or a fragment thereof. In an aspect, a disclosed MFGE8 can comprise a sequence having at least 70%, at least 75%, at least 80%, at least 85%, at least 90%, or at least 95% identity to the sequence set forth in SEQ ID NO:23 or a fragment thereof. In an aspect, a disclosed AGER can comprise the amino acid sequence set forth in SEQ ID NO:24 or a fragment thereof. In an aspect, a disclosed AGER can comprise a sequence having at least 70%, at least 75%, at least 80%, at least 85%, at least 90%, or at least 95% identity to the sequence set forth in SEQ ID NO:24 or a fragment thereof. In an aspect, a disclosed AGER can comprise the amino acid sequence set forth in SEQ ID NO:25 or a fragment thereof. In an aspect, a disclosed AGER can comprise a sequence having at least 70%, at least 75%, at least 80%, at least 85%, at least 90%, or at least 95% identity to the sequence set forth in SEQ ID NO:25 or a fragment thereof. In an aspect, a disclosed PROS1 can comprise the amino acid sequence set forth in SEQ ID NO:26 or a fragment thereof. In an aspect, a disclosed PROS1 can comprise a sequence having at least 70%, at least 75%, at least 80%, at least 85%, at least 90%, or at least 95% identity to the sequence set forth in SEQ ID NO:26 or a fragment thereof. In an aspect, a disclosed STAB1 can comprise the amino acid sequence set forth in SEQ ID NO:27 or a fragment thereof. In an aspect, a disclosed STAB1 can comprise a sequence having at least 70%, at least 75%, at least 80%, at least 85%, at least 90%, or at least 95% identity to the sequence set forth in SEQ ID NO:27 or a fragment thereof. In an aspect, a disclosed STAB1 can comprise the amino acid sequence set forth in SEQ ID NO:28 or a fragment thereof. In an aspect, a disclosed STAB1 can comprise a sequence having at least 70%, at least 75%, at least 80%, at least 85%, at least 90%, or at least 95% identity to the sequence set forth in SEQ ID NO:28 or a fragment thereof. In an aspect, a disclosed STAB2 can comprise the amino acid sequence set forth in SEQ ID NO:29 or a fragment thereof. In an aspect, a disclosed STAB2 can comprise a sequence having at least 70%, at least 75%, at least 80%, at least 85%, at least 90%, or at least 95% identity to the sequence set forth in SEQ ID NO:29 or a fragment thereof. In an aspect, a disclosed STAB2 can comprise the amino acid sequence set forth in SEQ ID NO:30 or a fragment thereof. In an aspect, a disclosed STAB2 can comprise a sequence having at least 70%, at least 75%, at least 80%, at least 85%, at least 90%, or at least 95% identity to the sequence set forth in SEQ ID NO:30 or a fragment thereof.

In an aspect, a disclosed HAVCR1 can comprise the amino acid sequence set forth in SEQ ID NO:31 or a fragment thereof. In an aspect, a disclosed HAVCR1 can comprise a sequence having at least 70%, at least 75%, at least 80%, at least 85%, at least 90%, or at least 95% identity to the sequence set forth in SEQ ID NO:31 or a fragment thereof. In an aspect, a disclosed HAVCR1 can comprise the amino acid sequence set forth in SEQ ID NO:32 or a fragment thereof. In an aspect, a disclosed HAVCR1 can comprise a sequence having at least 70%, at least 75%, at least 80%, at least 85%, at least 90%, or at least 95% identity to the sequence set forth in SEQ ID NO:32 or a fragment thereof. In an aspect, a disclosed HAVCR2 can comprise the amino acid sequence set forth in SEQ ID NO:33 or a fragment thereof. In an aspect, a disclosed HAVCR2 can comprise a sequence having at least 70%, at least 75%, at least 80%, at least 85%, at least 90%, or at least 95% identity to the sequence set forth in SEQ ID NO:33 or a fragment thereof. In an aspect, a disclosed HAVCR2 can comprise the amino acid sequence set forth in SEQ ID NO:34 or a fragment thereof. In an aspect, a disclosed HAVCR2 can comprise a sequence having at least 70%, at least 75%, at least 80%, at least 85%, at least 90%, or at least 95% identity to the sequence set forth in SEQ ID NO:34 or a fragment thereof. In an aspect, a disclosed TIMD4 can comprise the amino acid sequence set forth in SEQ ID NO:35 or a fragment thereof. In an aspect, a disclosed TIMD4 can comprise a sequence having at least 70%, at least 75%, at least 80%, at least 85%, at least 90%, or at least 95% identity to the sequence set forth in SEQ ID NO:35 or a fragment thereof. In an aspect, a disclosed TIMD4 can comprise the amino acid sequence set forth in SEQ ID NO:36 or a fragment thereof. In an aspect, a disclosed TIMD4 can comprise a sequence having at least 70%, at least 75%, at least 80%, at least 85%, at least 90%, or at least 95% identity to the sequence set forth in SEQ ID NO:36 or a fragment thereof. In an aspect, a disclosed PRKCA can comprise the amino acid sequence set forth in SEQ ID NO:37 or a fragment thereof. In an aspect, a disclosed PRKCA can comprise a sequence having at least 70%, at least 75%, at least 80%, at least 85%, at least 90%, or at least 95% identity to the sequence set forth in SEQ ID NO:37 or a fragment thereof. In an aspect, a disclosed PRKCA can comprise the amino acid sequence set forth in SEQ ID NO:38 or a fragment thereof. In an aspect, a disclosed PRKCA can comprise a sequence having at least 70%, at least 75%, at least 80%, at least 85%, at least 90%, or at least 95% identity to the sequence set forth in SEQ ID NO:38 or a fragment thereof. In an aspect, a disclosed SYT1 can comprise the amino acid sequence set forth in SEQ ID NO:39 or a fragment thereof. In an aspect, a disclosed SYT1 can comprise a sequence having at least 70%, at least 75%, at least 80%, at least 85%, at least 90%, or at least 95% identity to the sequence set forth in SEQ ID NO:39 or a fragment thereof. In an aspect, a disclosed SYT1 can comprise the amino acid sequence set forth in SEQ ID NO:40 or a fragment thereof. In an aspect, a disclosed SYT1 can comprise a sequence having at least 70%, at least 75%, at least 80%, at least 85%, at least 90%, or at least 95% identity to the sequence set forth in SEQ ID NO:40 or a fragment thereof. In an aspect, a disclosed JMJD6 can comprise the amino acid sequence set forth in SEQ ID NO:41 or a fragment thereof. In an aspect, a disclosed JMJD6 can comprise a sequence having at least 70%, at least 75%, at least 80%, at least 85%, at least 90%, or at least 95% identity to the sequence set forth in SEQ ID NO:41 or a fragment thereof.

In an aspect, a disclosed KLRK1 can comprise the amino acid sequence set forth in SEQ ID NO:102 or a fragment thereof. In an aspect, a disclosed KLRK1 can comprise a sequence having at least 70%, at least 75%, at least 80%, at least 85%, at least 90%, or at least 95% identity to the sequence set forth in SEQ ID NO:102 or a fragment thereof. In an aspect, a disclosed KLRK1 can comprise the amino acid sequence set forth in SEQ ID NO:103 or a fragment thereof. In an aspect, a disclosed KLRK1 can comprise a sequence having at least 70%, at least 75%, at least 80%, at least 85%, at least 90%, or at least 95% identity to the sequence set forth in SEQ ID NO:103 or a fragment thereof.

| Binding | |
|---|---|
| Domain | Identifying Information |
| ANXA1 | HGNC: 533/NCBI Entrez Gene: 301/Ensembl: ENSG00000135046/ |
| | OMIM: 151690/UniProtKB/Swiss-Prot: P04083 |
| ANXA2 | HGNC: 537/NCBI Entrez Gene: 302/Ensembl: ENSG00000182718/ |
| | OMIM: 151740/UniProtKB/Swiss-Prot: P07355) |
| ANXA3 | HGNC: 541/NCBI Entrez Gene: 306/Ensembl: ENSG00000138772/ |
| | OMIM: 106490/UniProtKB/Swiss-Prot: P12429 |
| ANXA4 | HGNC: 542/NCBI Entrez Gene: 307/Ensembl: ENSG00000196975/ |
| | OMIM: 106491/UniProtKB/Swiss-Prot: P09525 |
| ANXA5 | HGNC: 543/NCBI Entrez Gene: 308/Ensembl: ENSG00000164111/ |
| | OMIM: 131230/UniProtKB/Swiss-Prot: P08758 |
| ANXA6 | HGNC: 544/NCBI Entrez Gene: 309/Ensembl: ENSG00000197043/ |
| | OMIM: 114070/UniProtKB/Swiss-Prot: P08133 |
| ANXA7 | HGNC: 545/NCBI Entrez Gene: 310/Ensembl: ENSG00000138279/ |
| | OMIM: 186360/UniProtKB/Swiss-Prot: P20073 |
| ANXA8 | HGNC: 546/NCBI Entrez Gene: 653145/Ensembl: ENSG00000265190/ |
| | OMIM: 602396/UniProtKB/Swiss-Prot: P13928 |
| ANX8L1 | HGNC: 23334/NCBI Entrez Gene: 728113/Ensembl: ENSG00000264230/ |
| | UniProtKB/Swiss-Prot: Q5VT79 |
| ANXA9 | HGNC: 547/NCBI Entrez Gene: 8416/Ensembl: ENSG00000143412/ |
| | OMIM: 603319/UniProtKB/Swiss-Prot: O76027 |
| ANXA10 | HGNC: 534/NCBI Entrez Gene: 11199/Ensembl: ENSG00000109511/ |
| | OMIM: 608008/UniProtKB/Swiss-Prot: Q9UJ72 |
| ANXA11 | HGNC: 535/NCBI Entrez Gene: 311/Ensembl: ENSG00000122359/ |
| | OMIM: 602572/UniProtKB/Swiss-Prot: P50995 |
| ANXA13 | HGNC: 536/NCBI Entrez Gene: 312/Ensembl: ENSG00000104537/ |
| | OMIM: 602573/UniProtKB/Swiss-rot: P27216 |
| F2 | HGNC: 3535/NCBI Entrez Gene: 2147/Ensembl: ENSG00000180210/ |
| | OMIM: 176930/UniProtKB/Swiss-Prot: P00734) |
| F7 | (HGNC: 3544/NCBI Entrez Gene: 2155/Ensembl: ENSG00000057593/ |
| | OMIM: 613878/UniProtKB/Swiss-Prot: P08709) |
| F9 | (HGNC: 3551/NCBI Entrez Gene: 2158/Ensembl: ENSG00000101981/ |
| | OMIM: 300746/UniProtKB/Swiss-Prot: P00740) |
| F10 | (HGNC: 3528/NCBI Entrez Gene: 2159/Ensembl: ENSG00000126218/ |
| | OMIM: 613872/UniProtKB/Swiss-Prot: P00742) |
| AGER/ | (HGNC: 320 NCBI/Entrez Gene: 177/Ensembl: ENSG00000204305/ |
| RAGE | OMIM: 600214/UniProtKB/Swiss-Prot: Q15109 |
| GAS6 | HGNC: 4168 NCBI/Entrez Gene: 2621/Ensembl: ENSG00000183087/ |
| | OMIM: 600441/UniProtKB/Swiss-Prot: Q14393 |
| MFGE8 | HGNC: 7036 NCBI Entrez Gene: 4240/Ensembl: ENSG00000140545/ |
| | OMIM: 602281/UniProtKB/Swiss-Prot: Q08431 |
| AGCR/ | HGNC: 943/NCBI Entrez Gene: 575/Ensembl: ENSG00000181790/ |
| BAI1 | OMIM: 602682/UniProtKB/Swiss-Prot: O14514) |
| PROS | HGNC: 9456 NCBI Entrez Gene: 5627 Ensembl: ENSG00000184500 |
| | OMIM: 176880/UniProtKB/Swiss-Prot: P07225 |
| HAVCR1 | HGNC: 17866/NCBI Entrez Gene: 26762/Ensembl: ENSG00000113249/ |
| | OMIM: 606518/UniProtKB/Swiss-Prot: Q96D42 |
| HAVCR1 | HGNC: 18437/NCBI Entrez Gene: 84868 Ensembl: ENSG00000135077/ |
| | OMIM: 606652/UniProtKB/Swiss-Prot: Q8TDQ0 |
| TIMD4 | HGNC: 25132/NCBI Entrez Gene: 91937 Ensembl: ENSG00000145850/ |
| | OMIM: 610096/UniProtKB/Swiss-Prot: Q96H15 |
| PKCα | HGNC: 9393/NCBI Entrez Gene: 5578 Ensembl: ENSG00000154229/ |
| | OMIM: 176960/UniProtKB/Swiss-Prot: P17252 |
| SYT1 | HGNC: 11509/NCBI Entrez Gene: 6857 Ensembl: ENSG00000067715/ |
| | OMIM: 185605 UniProtKB/Swiss-Prot: P21579 |
| JMJD6 | HGNC: 19355/NCBI Entrez Gene: 23210 Ensembl: ENSG00000070495 |
| | OMIM: 604914 UniProtKB/Swiss-Prot: Q6NYC1 |
| APO-H | HGNC: 616/NCBI Entrez Gene: 350/Ensembl: ENSG00000091583/ |
| | OMIM: 138700/UniProtKB/Swiss-Prot: P02749 |
| STAB1 | HGNC: 18628/NCBI Entrez Gene: 23166/Ensembl: ENSG00000010327/ |
| | OMIM: 608560/UniProtKB/Swiss-Prot: Q9NY15 |
| STAB2 | HGNC: 18629/NCBI Entrez Gene: 55576/Ensembl: ENSG00000136011/ |
| | OMIM: 608561/UniProtKB/Swiss-Prot: Q8WWQ8 |
| KLRK1 | HGNC: 18788/NCBI Entrez Gene: 22914/Ensembl: ENSG00000213809/ |
| | OMIM: 611817/UniProtKB/Swiss-Prot: P26718 |

In an aspect, a disclosed PS binding domain can comprise the single-chain variable domain of bavituximab. In an aspect, a disclosed PS binding domain can comprise the sequence set forth in SEQ ID NO:55 or a fragment thereof. In an aspect, a disclosed PS binding chain can comprise a sequence having at least 70%, at least 75%, at least 80%, at least 85%, at least 90%, or at least 95% identity to the sequence set forth in SEQ ID NO:55 or a fragment thereof. In an aspect, a disclosed PS binding domain can comprise the single-chain variable domain of PGN632. In an aspect, a single-chain variable domain of PGN632 can comprise a γ1 heavy chain and a λ light chain. In an aspect, a single-chain variable domain of PGN632 can bind to cardiolipin/PS. In an aspect, a disclosed PS binding domain can comprise the single-chain variable domain of P1. In an aspect, a single-chain variable domain of P1 can comprise a γ1 heavy chain and a λ light chain. In an aspect, a single-chain variable domain of P1 can bind to cardiolipin/PS. In an aspect, a disclosed PS binding domain can comprise the single-chain variable domain of IS4. In an aspect, a single-chain variable domain of IS4 can comprise a γ3 VH1 heavy chain and a λ Vλ2 light chain. In an aspect, a single-chain variable domain of IS4 can bind to cardiolipin/PS. In an aspect, a disclosed PS binding domain can comprise the single-chain variable domain of CL1. In an aspect, a single-chain variable domain of CLL can comprise a γ3 VH1 heavy chain and a λ V3 light chain. In an aspect, a single-chain variable domain of IS4 can bind to cardiolipin/PS. In an aspect, PGN632, P1, IS4, and CLL are described in Moody et al. (2010) J. Exp. Med. 207(4):763-776, which is incorporated herein by reference for its teachings of these antibodies and their characteristics.

In an aspect of a disclosed chimeric fusion protein, a disclosed PS binding domain can be attached, linked, and/or conjugated to a disclosed immunostimulatory domain by any operative means. In an aspect, a disclosed operative means can comprise a direct covalent bond (e.g., a chemical cross-linker). In an aspect, a disclosed operative means can comprise direct attachment (e.g., avidin:biotin).

In an aspect, a disclosed PS binding domain protein and the operatively attached disclosed immunostimulatory domain can retain functionality. For example, the disclosed PS binding domain protein operatively linked to the disclosed immunostimulatory domain can retain one or more desired effector functions including, for example, pro-apoptotic and/or anti-angiogenic functions.

In an aspect, the disclosed PS binding domain protein operatively linked to the disclosed immunostimulatory domain can retain one or more desired effector functions including, for example, pro-apoptotic and/or anti-angiogenic functions. In an aspect, a disclosed PS binding domain protein operatively linked to the disclosed immunostimulatory domain can stimulate host effector functions, can target phosphatidylserine on tumor cells (including dividing, injured, and/or stressed), can localize to targeted cells and/or targeted sites (such as a hematological cancer cell), can exert any therapeutic effect (e.g., anti-cancer and/or anti-viral effects), or any combination thereof.

In an aspect, a disclosed linker can comprise a flexible linker. In an aspect, a flexible linker can comprise (GGGGS)n, wherein n=1-8 (SEQ ID NO:109-SEQ ID NO:116). In an aspect, a flexible linker can comprise (G)n, wherein n=1-8 (SEQ ID NO: 117-SEQ ID NO:124). In an aspect, a disclosed linker can comprise a rigid linker. In an aspect, a flexible linker can comprise (EAAAK)n, wherein n=1-8 (SEQ ID NO:125-SEQ ID NO:132). In an aspect, a disclosed linker can comprise a Whitlow linker. In an aspect, a Whitlow linker can comprise (GSTSGSGKSSEGKG)n, wherein n=1-8 (SEQ ID NO:133-SEQ ID NO:140). In an aspect, a Whitlow linker can comprise (GSTSGSGKPGSGEGSTKG)n, wherein n=1-8 (SEQ ID NO:141-SEQ ID NO:148). Linkers are known to the skilled person in the art.

In an aspect of a chimeric fusion protein, a disclosed immunostimulatory domain can be attached, linked, and/or conjugated by any operative means to a disclosed PS binding domain.

In an aspect, a disclosed immunostimulatory domain can comprise a Fc domain of an immunoglobulin. In an aspect, a disclosed immunoglobulin can be a human immunoglobulin. In an aspect, a disclosed human immunoglobulin can be IgG1, IgG2, IgG3, IgG4, or any combination thereof. In an aspect, a disclosed immunostimulatory domain can comprises the Fc domain of IgG1. In an aspect, a disclosed immunostimulatory domain can comprises the Fc domain of IgG2. In an aspect, a disclosed immunostimulatory domain can comprise the Fc domain of IgG3. In an aspect, a disclosed immunostimulatory domain can comprise the Fc domain of IgG4.

In an aspect, a disclosed chimeric fusion protein can comprise a PS-binding domain operably linked to the Fc domain of IgG1. In an aspect, a disclosed chimeric fusion protein can comprise a PS-binding domain operably linked to the Fc domain of IgG2. In an aspect, a disclosed chimeric fusion protein can comprise a PS-binding domain operably linked to the Fc domain of IgG3. In an aspect, a disclosed chimeric fusion protein can comprise a PS-binding domain operably linked to the Fc domain of IgG4.

In an aspect, a disclosed chimeric fusion protein can comprise a PS-binding domain operably linked to the Fc domain of human IgG1. In an aspect, a disclosed chimeric fusion protein can comprise a PS-binding domain operably linked to the Fc domain of human IgG2. In an aspect, a disclosed chimeric fusion protein can comprise a PS-binding domain operably linked to the Fc domain of human IgG3. In an aspect, a disclosed chimeric fusion protein can comprise a PS-binding domain operably linked to the Fc domain of human IgG4.

In an aspect, a disclosed IgG1 can comprise the sequence set forth in SEQ ID NO:84 or a fragment thereof. In an aspect, a disclosed IgG1 can comprise a sequence having at least 70%, at least 75%, at least 80%, at least 85%, at least 90%, or at least 95% identity to the sequence set forth in SEQ ID NO:84 or a fragment thereof.

In an aspect, a disclosed immunostimulatory domain can comprise a single-chain antibody (scFv) to a human CD3 protein. The CD3 antigen is a surface structure associated with the T-cell receptor (TCR) to form a complex involved in antigen recognition and signal transduction. CD3 comprises four distinct chains. In mammals, the CD3 complex comprises a CD3γ chain, a CD3δ chain, and two CD3ε chains. These chains associate with the T-cell receptor (TCR) and the CD3-zeta (ζ-chain) to generate an activation signal in T lymphocytes. The TCR, CD3-zeta, and the other CD3 molecules together constitute the TCR complex. In an aspect, an anti-CD3 antibody can comprise a OKT3 or humanized OKT3 antibody. In an aspect, an anti-CD3 anti-body can comprise OKT3 (muromab), ChAglyCD3 (otelixizumab), hOKT3γ1 (teplizumab), nuvion (visilizumab), NI-0401 (foralumab), a UCHT1 or humanized UCHT1 antibody, tepilizumab, TRX4 (otlixizumab), or any combination thereof. In an aspect, a disclosed immunostimulatory domain can comprise one or more anti-CD3 antibodies.

In an aspect, a disclosed bi-specific chimeric fusion protein can comprise a disclosed PS-binding domain and an anti-CD marker for hematological cancer (e.g., CD1a, CD2, CD3, CD4, CD5, CD7, CD8, CD9, CD10, CD11b, CD11c, CD13, CD14, CD15, CD16, CD19, CD20, CD22, CD23, CD25, CD26, CD30, CD33, CD34, CD36, CD38, CD41, CD43, CD45, CD45RA, CD45RO, CD52, CD56, CD58, CD61, CD64, CD71, CD79a, CD79b, CD103, CD117, CD123, CD138, or CD163).

In an aspect, a disclosed PS-binding domain operably linked to an Fc domain can be fused to an additional immunostimulatory domain. In an aspect, a disclosed additional immunostimulatory domain can be any disclosed immunostimulatory domain. In an aspect, a disclosed additional immunostimulatory domain can be fused, attached, linked, and/or conjugated by any operative means to a disclosed PS-binding domain operably linked to an Fc domain. In an aspect, a disclosed additional immunostimulatory domain can comprise a Fc domain of an immunoglobulin. In an aspect, a disclosed additional immunoglobulin can be a human immunoglobulin. In an aspect, a disclosed additional human immunoglobulin can be IgG1, IgG2, IgG3, IgG4, or any combination thereof. In an aspect, a disclosed additional immunostimulatory domain can comprises the Fc domain of IgG1. In an aspect, a disclosed additional immunostimulatory domain can comprises the Fc domain of IgG2. In an aspect, a disclosed additional immunostimulatory domain can comprise the Fc domain of IgG3. In an aspect, a disclosed additional immunostimulatory domain can comprise the Fc domain of IgG4.

In an aspect, a disclosed chimeric fusion protein can comprise a PS-binding domain operably linked to the Fc domain of IgG1. In an aspect, a disclosed chimeric fusion protein can comprise a PS-binding domain operably linked to the Fc domain of IgG2. In an aspect, a disclosed chimeric fusion protein can comprise a PS-binding domain operably linked to the Fc domain of IgG3. In an aspect, a disclosed chimeric fusion protein can comprise a PS-binding domain operably linked to the Fc domain of IgG4.

In an aspect, a disclosed chimeric fusion protein can comprise a homozygous dimer of a disclosed PS-binding domain linked to an scFv against CD3, which can be fused with an Fc domain. In an aspect, a disclosed chimeric fusion protein can comprise a homozygous dimer of a disclosed PS-binding domain linked to an scFv against CD3 connected through a Fc hinge region (but not a Fc domain). In an aspect, a disclosed chimeric fusion protein can comprise a disclosed PS-binding domain linked to an scFv against CD3, which can be fused with an Fc domain only. In an aspect, a disclosed chimeric fusion protein can comprise a heterozygous dimer of a PS-binding domain and an anti-CD3 scFv but not a Fc domain. In an aspect, a disclosed chimeric fusion protein can comprise a heterozygous dimer of a PS-binding domain and an anti-CD3 scFv fused with Fc domain.

In an aspect, a disclosed chimeric fusion protein can further comprise a conjugated and/or fused small molecule. In an aspect, a disclosed small molecule can be a disclosed tubulin interactor, a disclosed DNA modifier, a disclosed RNA inhibitor, or any combination thereof. In an aspect, a disclosed small molecule can be a disclosed tubulin interactor, a disclosed DNA modifier, a disclosed RNA inhibitor, or any combination thereof.

In an aspect, a disclosed tubulin interactor can comprise a maytansinoid or an auristatin. In an aspect, a disclosed maytansinoid can be a class of microtubulin polymerization inhibitors derived from the naturally occurring maytansine (e.g., a benzoansamacrolide that is isolated from the bark of the African shrub Maytenus ovatus). In an aspect, maytansine can bind tubulin at the vinca-binding site, similar to vinca alkaloids, and can thereby depolymerizing tubulin and inducing mitotic arrest. In an aspect, a disclosed synthetic maytansine derivatives can comprise a 100-1000-fold increase in potency over a drug maytansinoid (DMs). In an aspect, a disclosed maytansine derivative can comprise a modification enabling conjugation to an antibody. In an aspect, a disclosed maytansinoid-based ADC can be trastuzumab emtansine. Auristatins are a family of complex analogues to the native antineoplastic product dolastatin 10. In an aspect, auristatins can be 100 to 1000 times more toxic than doxorubicin. In an aspect, auristatins can inhibit microtubule formation through the interaction with tubulin at the “peptide sub-site” of tubulin's “Vinca domain” and can disrupt tubulin-dependent GTP hydrolysis. In an aspect, auristatins can lead to the arrest of cancer cells in the mitosis stage and can eventually elicit apoptosis. In an aspect, a disclosed auristatin-based ADC can comprise brentuximab vedotin (SGN-35), polatuzumab vedotin (RG7596/DCDS-4501 A), enfortumab vedotin (AGS-22ME, telisotuzumab vedotin (ABBV-399), tisotumab vedotin (HuMax-TF-ADC), anti-PSMA ADC, ladiratuzumab vedotin (SGN-LIV1A), enapotamab vedotin (HuMax-Axl-ADC), AGS-16C3F, GSK-2857916, or any combination thereof.

In an aspect, a disclosed ADC payload can a DNA modifier. In an aspect, a disclosed DNA modifier can comprise a calicheamicin, a duocarmycin, or a camptothecin. In an aspect, calicheamicins are a family of enediyne antitumor antibiotics that were isolated from the fermentation broth of Micromonospora echinospora. In an aspect, a disclosed calicheamicin can cause strand scission of the DNA through a reaction similar to the Bergman cyclization that generates a diradical species which abstracts a proton from the sugar backbone, the process being initiated by nucleophilic attack on the central atom of the trisulphide. In an aspect, calicheamicin can be approximately 4000 times more potent than doxorubicin. In an aspect, duocarmycins are another class of DNA-alkylating agents that bind the minor groove of DNA and alkylate the adenine residues at the N3 position. Duocarmycin analogues, among which CC-1065 and duocarmycin SA are the most widely used, represent a series of extremely powerful antineoplastic compounds that display high cytotoxicity against the growing cancer cells in culture. Duocarmycin analogues are DNA minor groove binding agents that also exert adenine-N3 alkylation activity and an AT-sequence selectivity. In an aspect, duocarmycin analogues can bind the minor groove of DNA and then can induce irreversible DNA alkylation that hinders DNA architecture and structural integrity. The alkylation of DNA eventually leads to DNA cleavage and subsequently, tumor cell death via apoptosis. In an aspect, duocarmycins can cleave DNA at any cellular cycle. In an aspect, duocarmycin analogues can be effective on solid tumors.

In an aspect, camptothecin is a cytotoxic plant alkaloid originally isolated from C. acuminate that can inhibit DNA and RNA synthesis in mammalian cells and can be an effective anti-tumor agent. Camptothecin can inhibit topoisomerase I with an IC₅₀ of 679 nM. In an aspect, camptothecin can bind and can stabilize topoisomerase I-DNA cleavage complexes, which can lead to DNA strand breaks. The resultant DNA damage can induce cell cycle arrest in many cancer cell lines. In an aspect, inactivation of the tumor suppressor protein p53 can increase the cytotoxicity of camptothecin.

In an aspect, a disclosed ADC can be SN38 (7-ethyl-10-hydroxy-camptothecin). In an aspect, SN-38 can bind to and can inhibit topoisomerase I by stabilizing the cleavable complex between topoisomerase I and DNA, thereby resulting in DNA breaks, inhibition of DNA replication, and apoptosis. SN-38 can exhibit up to 1,000-fold more cytotoxic activity against various cancer cells in vitro than irinotecan. In an aspect, SN38 can be a liposomal formulation. In an aspect, a liposocomal formulation of SN-38 can increase the solubility of SN-38 and can improve the pharmacodynamic profile as compared to SN-38 alone.

In an aspect, a disclosed ADC payload can comprise a RNA inhibitor. In an aspect, a disclosed RNA inhibitor can be an amatoxins.

Amatoxins are bicyclic octapeptides of which alpha, beta, and gamma amatoxins are the most powerful toxins. The molecular weight of an amatoxin can be about 900 Da. In an aspect, amatoxins can be heat stable and can remain toxic after prolonged periods of storage. In an aspect, amatoxins can inhibit RNA polymerase II function thereby disrupting DNA and RNA transcription. In an aspect, a disclosed ADC can be FDA approved, for example, gemtuzumab ozogamicin, inotuzumab ozogamicin, ado-trastuzumab emtansine, brentuximab vedotin), polatuzumab vedotin, or any combination thereof.

In an aspect, a disclosed ADC can be a STING (i.e., stimulator of interferon (IFN) genes) agonist and/or a STING-targeting compound. In an aspect, STING is an endoplasmic protein that can induce the production of pro-inflammatory cytokines such as type I IFNs. STING is a transmembrane protein localized to the endoplasmic reticulum and functions as an adaptor protein in the cGAS (cyclic GMP-AMP synthase)-STING pathway. cGAS-STING is a cytosolic DNA-sensing pathway that drives activation of type I IFN and other inflammatory cytokines in the host immune response against tumors. Recognition of cytoplasmic tumor-derived DNA by c-GAS can generate cGAMPs (e.g., natural ligands of STING protein). The binding of cGAMP to STING can induce transformational changes in STING protein, activating a downstream signaling cascade involving TBK1 and IRF-3, which results in the production of type I IFNs. In an aspect, a disclosed STING agonist can be DMXAA, ASA404, ADU-S100/MIW815, MK-1454, MK-2118, SB11285, GSK3745417, BMS-986301, BI-STING (BI 1387446), E7766, TAK-676, SNX281, JNJ-67544412 (JNJ-4412), BI-STING, 3′3′-cyclic 3′3′-cAIMP, GSK532, Ryvu's agonists, GF3-002, Selvita agonists, TTI-10001, JNJ-6196, CRD5500, CS-1018, CS-1020, CS-1010, MSA-1, ALG-031048, E7766, SR-8541A, SR-8314, oral ENPP1 inhibitors, MV-626, or any combination thereof. In an aspect, a disclosed STING-targeting compound can be a cyclic dinucleotide (CDN), a non-CDN agonist, a macrocyclic STING agonist, an ENPP1 inhibitor, or any combination thereof.

In an aspect, a disclosed conjugated and/or fused small molecule can be an immune-stimulatory protein. In an aspect, a disclosed immune-stimulatory protein can be a SIRPα (IgV domain) or a subdomain thereof. SIRPα, also known as CD172a or Src homology 2 (SH2) domain-containing phosphatase substrate-1, is a cell surface receptor expressed predominantly in monocytes, granulocytes, dendritic cells and hematopoietic stem cell. In an aspect, a disclosed subdomain of SIRPα can bind CD47. CD47, an SIRPα ligand, has been described as an important anti-phagocytic signal expressed on tumor cells. In an aspect, a disclosed signal regulatory protein (SIRP) family comprises an inhibitory receptor, SIRPα, an activating receptor, SIRP3 (also known as cluster of differentiation (CD)172b), and a non-signaling receptor, SIRP7 (also known as CD172g). As known to the art, CD47 is a membrane protein expressed in nearly all cell types. The Ig domain of CD47 and the N-terminal Ig domain of SIRPα can bind directly. In an aspect, the binding of SIRPα to CD47 can promote tyrosine phosphorylation of the cytoplasmic region of SIRPα. In an aspect, a disclosed protein tyrosine phosphatase, SHP-2 (also known as tyrosine-protein phosphatase non-receptor type 11), can bind to the cytoplasmic region of SIRPα to mediate the functions of SIRPα by dephosphorylating its substrates. In an aspect, SIRPα can function as a negative signaling regulator. In an aspect, a disclosed chimeric fusion protein comprising a conjugated and/or fused SIRPα (IgV domain) or a subdomain thereof can comprise the sequence set forth in SEQ ID NO:99, SEQ ID NO:100, or SEQ ID NO:101.

In an aspect, a disclosed immune-stimulatory protein can comprise the extracellular domain of the NKG2D protein. KLRK1 (Killer Cell Lectin Like Receptor K1) is a protein-coding gene. Natural killer (NK) cells are lymphocytes that can mediate lysis of certain tumor cells and virus-infected cells without previous activation. They can also regulate specific humoral and cell-mediated immunity. NK cells preferentially express several calcium-dependent (C-type) lectins, which have been implicated in the regulation of NK cell function. The NKG2 gene family is located within the NK complex, a region that contains several C-type lectin genes preferentially expressed in NK cells. This gene encodes a member of the NKG2 family. The encoded transmembrane protein is characterized by a type II membrane orientation (has an extracellular C terminus) and the presence of a C-type lectin domain. It binds to a diverse family of ligands that include MHC class I chain-related A and B proteins and UL-16 binding proteins, where ligand-receptor interactions can result in the activation of NK and T cells. The surface expression of these ligands is important for the recognition of stressed cells by the immune system, and thus this protein and its ligands are therapeutic targets for the treatment of immune diseases and cancers. Read-through transcription exists between this gene and the upstream KLRC4 (killer cell lectin-like receptor subfamily C, member 4) family member in the same cluster. Diseases associated with KLRK1 include cowpox and immunodeficiency, X-Linked, with Magnesium Defect, Epstein-Barr Virus Infection, and neoplasia. Among its related pathways are DAP12 interactions and innate immune system. Gene Ontology (GO) annotations related to this gene include signaling receptor activity and carbohydrate binding. An important paralog of this gene is OLR1.

In an aspect, a disclosed conjugated and/or fused small molecule can be an immune-stimulatory protein.

In an aspect, a disclosed small molecule can be a disclosed tubulin interactor, a disclosed DNA modifier, a disclosed RNA inhibitor, or any combination thereof. In an aspect, a disclosed chimeric fusion protein comprising a conjugated and/or fused NKG2D can comprise the sequence set forth in SEQ ID NO:106.

In an aspect, a disclosed therapeutically effective amount or effective dose of a disclosed chimeric fusion protein or pharmaceutical formulation thereof can be about 0.01 μg/kg to about 10 mg/kg per subject.

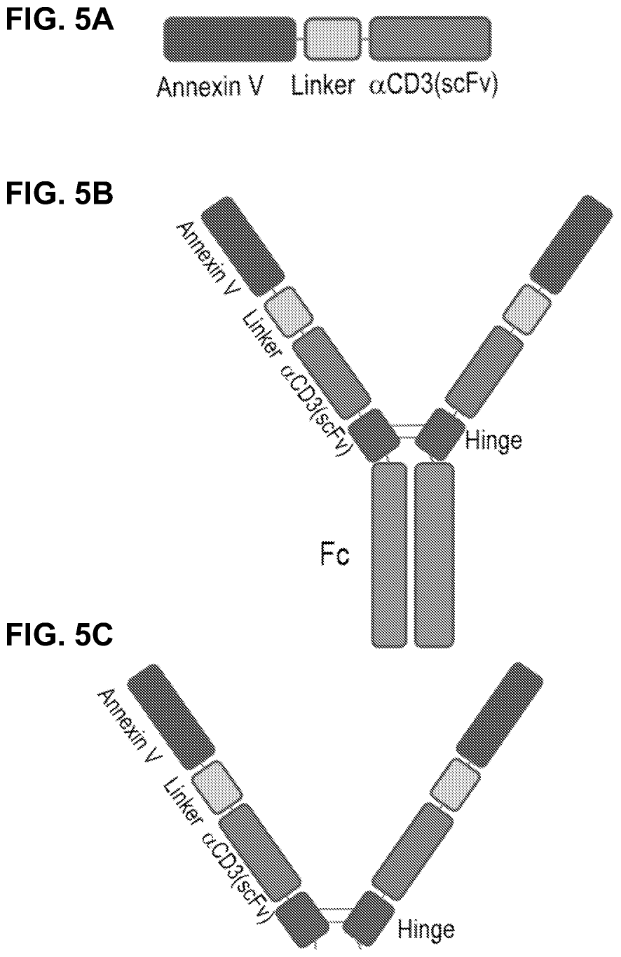

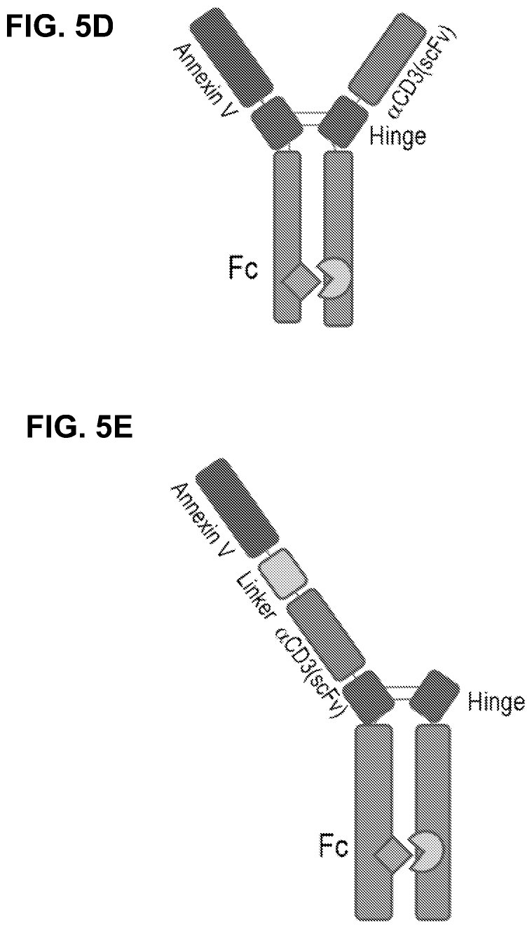

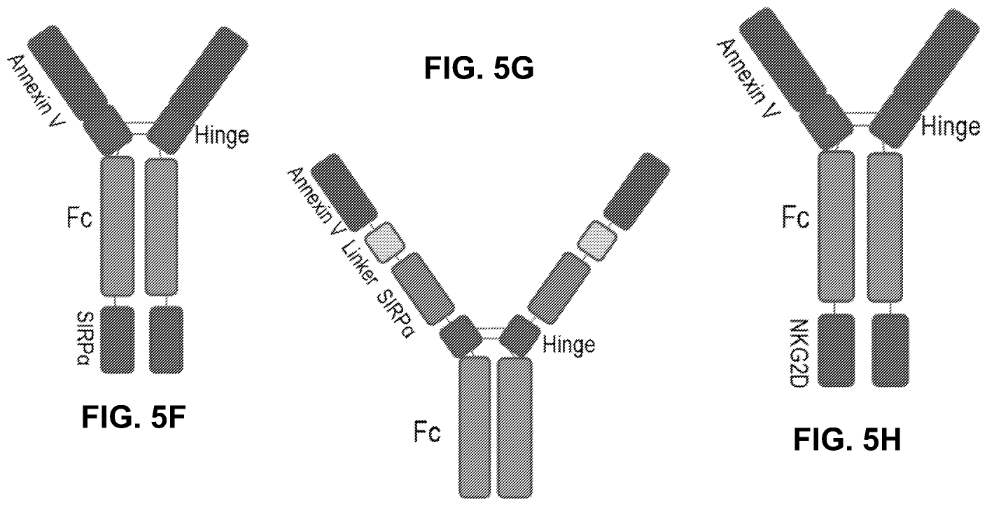

In an aspect, a disclosed chimeric fusion protein can comprise the structure set forth in FIG. 5A, FIG. 5B, FIG. 5C, FIG. 5D, FIG. 5E, FIG. 5F, or FIG. 5G. In an aspect, a disclosed chimeric fusion protein is bi-specific (e.g., PS binding domain and anti-CD3). In an aspect, a disclosed chimeric fusion protein can comprise the structure set forth in FIG. 5A, FIG. 5B, FIG. 5C, FIG. 5D, FIG. 5E, FIG. 5F, or FIG. 5G, wherein the PS-binding domain comprises any disclosed PS-binding domain and wherein the anti-CD3 binding domain comprises any disclosed binding domain. In an aspect, a disclosed bi-specific chimeric fusion protein can comprise a disclosed PS-binding domain and an anti-CD marker for hematological cancer (e.g., CD1a, CD2, CD3, CD4, CD5, CD7, CD8, CD9, CD10, CD11b, CD11c, CD13, CD14, CD15, CD16, CD19, CD20, CD22, CD23, CD25, CD26, CD30, CD33, CD34, CD36, CD38, CD41, CD43, CD45, CD45RA, CD45RO, CD52, CD56, CD58, CD61, CD64, CD71, CD79a, CD79b, CD103, CD117, CD123, CD138, or CD163).

In an aspect, a disclosed chimeric fusion protein can be part of an antibody drug conjugate (ADCs). In an aspect, a disclosed ADC payload can be restricted to highly potent cytotoxic molecules (pM to nM IC50) and can rely on delivery of these highly potent cytotoxic molecules to the targeted cells or tumor microenvironment. In an aspect, a disclosed ADC payload can be defined by its mechanism of action.

In an aspect, a disclosed ADC payload can be a tubulin interactor. In an aspect, a disclosed tubulin interactor can comprise a maytansinoids, or an auristatin. In an aspect, a disclosed chimeric fusion protein can induce cell death in the targeted cell. In an aspect, a disclosed chimeric fusion protein can stimulate an effector cell mediated immune modulator response to PS-expressing tumor cells. In an aspect, a disclosed chimeric fusion protein can induce a tumor reducing immune response. In an aspect, a disclosed chimeric fusion protein can induce phagocytosis of cancer cells in the subject. In an aspect, a disclosed chimeric fusion protein can cross-prime an anti-tumor T cell response. In an aspect, a disclosed chimeric fusion protein can induce a tumor eliminating immune response. In an aspect, a disclosed ADC can treat cancer. In an aspect, a disclosed chimeric fusion protein can improve and/or extend the survivability of the subject, can improve a subject's quality of life, can increase and/or prolong a subject's life span, or any combination thereof.

Disclosed herein is a chimeric fusion protein, comprising a phosphatidylserine (PS) binding domain, a linker, and an immunostimulatory domain. Disclosed herein is a chimeric fusion protein, comprising a phosphatidylserine (PS) binding domain operably linked to an immunostimulatory domain. Disclosed herein is a chimeric fusion protein, comprising a secretion signal and a phosphatidylserine (PS) binding domain operably linked to an immunostimulatory domain. Disclosed herein is a chimeric fusion protein comprising a phosphatidylserine (PS) binding domain operably linked to an immunostimulatory domain, wherein the immunostimulatory domain further comprise a conjugated small molecule.

In an aspect, a disclosed chimeric fusion protein can be part of an antibody drug conjugate (ADCs). In an aspect, a disclosed ADC can enable the delivery of cytotoxic payloads to PS-expressing cancer cells. In an aspect, a disclosed ADCs can act by directly targeting and then killing selected cells, affecting both the growth and behavior of cells. In an aspect, a disclosed ADC can comprise three parts including (i) a disclosed antibody specific to the target associated antigen (i.e., phosphatidylserine on a tumor cell), (ii) a payload designed to kill target cancer cells, and (iii) a chemical linker to attach the payload to the antibody

### 2. Antibody Drug Conjugates

Disclosed herein is an antibody drug conjugate comprising a disclosed chimeric fusion protein. Disclosed herein is an antibody drug conjugate comprising a disclosed chimeric fusion protein comprising a phosphatidylserine (PS) binding domain. Disclosed herein is an antibody drug conjugate comprising a disclosed chimeric fusion protein comprising a phosphatidylserine (PS) binding domain and an immunostimulatory domain. Disclosed herein is an antibody drug conjugate comprising a disclosed chimeric fusion protein comprising a phosphatidylserine (PS) binding domain operably linked an immunostimulatory domain. Disclosed herein is an antibody drug conjugate comprising a disclosed chimeric fusion protein comprising a phosphatidylserine (PS) binding domain, a linker, and an immunostimulatory domain. Disclosed herein is an antibody drug conjugate comprising a disclosed chimeric fusion protein comprising a phosphatidylserine (PS) binding domain operably linked to an immunostimulatory domain, wherein the immunostimulatory domain further comprise a conjugated small molecule. Disclosed herein is an antibody drug conjugate comprising a disclosed chimeric fusion protein comprising a secretion signal and a phosphatidylserine (PS) binding domain. Disclosed herein is an antibody drug conjugate comprising a disclosed chimeric fusion protein comprising a secretion signal, a phosphatidylserine (PS) binding domain, and an immunostimulatory domain. Disclosed herein is an antibody drug conjugate comprising a disclosed chimeric fusion protein comprising a secretion signal and a phosphatidylserine (PS) binding domain operably linked an immunostimulatory domain. Disclosed herein is an antibody drug conjugate comprising a disclosed chimeric fusion protein comprising a secretion signal, a phosphatidylserine (PS) binding domain, a linker, and an immunostimulatory domain. Disclosed herein is an antibody drug conjugate comprising a disclosed chimeric fusion protein comprising a secretion signal and a phosphatidylserine (PS) binding domain operably linked to an immunostimulatory domain, wherein the immunostimulatory domain further comprise a conjugated small molecule.

Disclosed herein is an antibody drug conjugate comprising a disclosed chimeric fusion protein comprising a PS-binding domain operably linked to an IgG1 Fc domain. Disclosed herein is an antibody drug conjugate comprising a disclosed chimeric fusion protein comprising a PS-binding domain operably linked to an IgG1 Fc domain and an additional immunostimulatory domain. Disclosed herein is an antibody drug conjugate comprising a disclosed chimeric fusion protein comprising a PS-binding domain operably linked to an IgG1 Fc domain and an additional immunostimulatory domain, wherein the additional immunostimulatory domain comprises the IgV domain of SIRPα or the extracellular domain of NKG2D protein. Disclosed herein is an antibody drug conjugate comprising a disclosed chimeric fusion protein comprising a PS-binding domain operably linked to a single-chain antibody (scFV) to a human CD3 protein. Disclosed herein is an antibody drug conjugate comprising a disclosed chimeric fusion protein comprising an Annexin-binding domain operably linked to a single-chain antibody (scFV) to a human CD3 protein. Disclosed herein is an antibody drug conjugate comprising a disclosed chimeric fusion protein comprising a PS-binding domain operably linked to a single-chain antibody (scFV) to a human CD3 protein and one or more additional immunostimulatory domains. Disclosed herein is an antibody drug conjugate comprising a disclosed chimeric fusion protein comprising an Annexin-binding domain operably linked to a single-chain antibody (scFV) to a human CD3 protein and one or more additional immunostimulatory domains.

Disclosed herein is an antibody-drug conjugate comprising a disclosed chimeric fusion protein comprising the sequence set forth in SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:99, SEQ ID NO:100, SEQ ID NO:101, SEQ ID NO:106, or a fragment thereof.

In an aspect, a disclosed antibody-drug conjugate can comprise a disclosed chimeric fusion protein comprising the structure set forth in FIG. 5A, FIG. 5B, FIG. 5C, FIG. 5D, FIG. 5E, FIG. 5F, or FIG. 5G. In an aspect, a disclosed chimeric fusion protein is bi-specific (e.g., PS binding domain and anti-CD3). In an aspect, a disclosed antibody-drug conjugate can comprise a disclosed chimeric fusion protein comprising the structure set forth in FIG. 5A, FIG. 5B, FIG. 5C, FIG. 5D, FIG. 5E, FIG. 5F, or FIG. 5G, wherein the PS-binding domain comprises any disclosed PS-binding domain and wherein the anti-CD3 binding domain comprises any disclosed binding domain. In an aspect, a disclosed bi-specific chimeric fusion protein can comprise a disclosed PS-binding domain and an anti-CD marker for hematological cancer (e.g., CD1a, CD2, CD3, CD4, CD5, CD7, CD8, CD9, CD10, CD11b, CD11c, CD13, CD14, CD15, CD16, CD19, CD20, CD22, CD23, CD25, CD26, CD30, CD33, CD34, CD36, CD38, CD41, CD43, CD45, CD45RA, CD45RO, CD52, CD56, CD58, CD61, CD64, CD71, CD79a, CD79b, CD103, CD117, CD123, CD138, or CD163).

In an aspect, a disclosed ADC can enable the delivery of cytotoxic payloads to PS-expressing cancer cells. In an aspect, a disclosed ADCs can act by directly targeting and then killing selected cells, affecting both the growth and behavior of cells. In an aspect, a disclosed ADC can comprise three parts including (i) a disclosed antibody specific to the target associated antigen (i.e., phosphatidylserine on a tumor cell), (ii) a payload designed to kill target cancer cells, and (iii) a linker to attach the payload to the antibody.

In an aspect of an ADC, a disclosed linker can be stable in circulation, but can release a disclosed cytotoxic agent in the targeted cells. In an aspect, a disclosed linker can be a noncleavable linker or can be cleavable linker.

In an aspect, a disclosed noncleavable linker can comprise a stable bonds that resists proteolytic degradation. In an aspect, a disclosed noncleavable linker can provide higher stability than a cleavable linker. In an aspect, a disclosed mechanism of action of a noncleavable linker can be based on the internalization of the ADC complex followed by degradation of the mAb component in the lysosome, thereby resulting in the release of a cytotoxic drug that kills tumor cells. In an aspect, a disclosed noncleavable linker does not unleash cytotoxic agents at off-target sites and thus do not harm healthy cells. In an aspect, a disclosed noncleavable linker can make it possible to modify the chemical properties of small molecules to modulate the affinity of the transporter and to improve the efficiency.

In an aspect, a disclosed cleavable linker can be cleaved by environmental differences (such as redox potential, pH) and specific lysosomal enzymes in response to extracellular and intracellular environments. In an aspect, a disclosed cleavable linker can be an acid-sensitive linker or an acid-labile linker. In an aspect, cleavable linkers can comprise IMMU-100.

In an aspect, a disclosed cleavable linker can be a lysosomal protease-sensitive linker. In an aspect, a disclosed lysosomal protease-sensitive linker can comprise a valine-citrulline linker, a valine-alanine linker, or a phenylalanine-lysine linker.

In an aspect, a disclosed cleavable linker can be a β-glucuronide linker. In an aspect, a disclosed β-glucuronide linker can be recognized and hydrolyzed by β-glucuronidase for payload release. In an aspect, a disclosed cleavable linker can be a glutathione-sensitive disulfide linker. In an aspect, a disclosed β-glucuronide linker can be recognized and hydrolyzed by β-glucuronidase for payload release.

In an aspect, a disclosed linker can be a nonreducible thioether linkage (SMCC). In an aspect, a disclosed linker can be a nonreducible thioether linker MCC (4-[N-maleimidomethyl]cyclohexane-1-carboxylate).

In an aspect, a disclosed antibody specific to the target-associated antigen can comprise a disclosed chimeric fusion protein. In an aspect, a disclosed antibody specific to the target-associated antigen can be specific for a PS-expressing hematological cancer cell. In an aspect, a disclosed antibody can be specific to the target-associated antigen with restricted or no expression on normal and/or healthy cells.

In an aspect, a disclosed ADC can comprise a potent cytotoxic agent. In an aspect, a disclosed chimeric fusion protein can further comprise a conjugated and/or fused small molecule. In an aspect, a disclosed small molecule can be a disclosed tubulin interactor, a disclosed DNA modifier, a disclosed RNA inhibitor, or any combination thereof. In an aspect, a disclosed ADC payload can be restricted to highly potent cytotoxic molecules (pM to nM IC50) and can rely on delivery of these highly potent cytotoxic molecules to the targeted cells or tumor microenvironment.

In an aspect, a disclosed ADC payload can be defined by its mechanism of action. In an aspect, a disclosed ADC payload can be a tubulin interactor. In an aspect, a disclosed tubulin interactor can comprise a maytansinoids or an auristatin (discussed supra). In an aspect, a disclosed ADC payload can a DNA modifier. In an aspect, a disclosed DNA modifier can comprise a calicheamicin, a duocarmycin, or a camptothecin (discussed supra). In an aspect, a disclosed ADC payload can comprise an RNA inhibitor. In an aspect, a disclosed RNA inhibitor can be an amatoxin (discussed supra). In an aspect, a disclosed ADC can comprise a combination of chemotherapy and immunotherapy.

In an aspect, a disclosed ADC can induce cell death in the targeted cell. In an aspect, a disclosed ADC can stimulate an effector cell mediated immune modulator response to PS-expressing tumor cells. In an aspect, a disclosed ADC can induce a tumor reducing immune response. In an aspect, a disclosed ADC can induce phagocytosis of cancer cells in the subject. In an aspect, a disclosed ADC can cross-prime an anti-tumor T cell response. In an aspect, a disclosed ADC can induce a tumor eliminating immune response. In an aspect, a disclosed ADC can treat cancer. In an aspect, a disclosed ADC can improve and/or extend the survivability of the subject, can improve a subject's quality of life, can increase and/or prolong a subject's life span, or any combination thereof.

### 3. Isolated Nucleic Acid Molecules

Disclosed herein are isolated nucleic acid molecules comprising a nucleic acid sequence encoding one or more disclosed chimeric fusion proteins. Disclosed herein is an isolated nucleic acid molecule comprising a nucleic acid sequence encoding a chimeric fusion protein comprising a phosphatidylserine (PS) binding domain. Disclosed herein is an isolated nucleic acid molecule comprising a nucleic acid sequence encoding a chimeric fusion protein comprising a phosphatidylserine (PS) binding domain and an immunostimulatory domain. Disclosed herein is an isolated nucleic acid molecule comprising a nucleic acid sequence encoding a chimeric fusion protein comprising a phosphatidylserine (PS) binding domain operably linked an immunostimulatory domain. Disclosed herein is an isolated nucleic acid molecule comprising a nucleic acid sequence encoding a phosphatidylserine (PS) binding domain, a linker, and an immunostimulatory domain. Disclosed herein is an isolated nucleic acid molecule comprising a nucleic acid sequence encoding a chimeric fusion protein comprising a phosphatidylserine (PS) binding domain operably linked to an immunostimulatory domain, wherein the immunostimulatory domain further comprise a conjugated small molecule.

Disclosed herein is an isolated nucleic acid molecule comprising a nucleic acid sequence encoding a chimeric fusion protein comprising a secretion signal and a phosphatidylserine (PS) binding domain. Disclosed herein is an isolated nucleic acid molecule comprising a nucleic acid sequence encoding a chimeric fusion protein comprising a secretion signal, a phosphatidylserine (PS) binding domain, and an immunostimulatory domain. Disclosed herein is an isolated nucleic acid molecule comprising a nucleic acid sequence encoding a chimeric fusion protein comprising a secretion signal and a phosphatidylserine (PS) binding domain operably linked an immunostimulatory domain. Disclosed herein is an isolated nucleic acid molecule comprising a nucleic acid sequence encoding a chimeric fusion protein comprising a secretion signal, a phosphatidylserine (PS) binding domain, a linker, and an immunostimulatory domain.

Disclosed herein is an isolated nucleic acid molecule comprising a nucleic acid sequence encoding a chimeric fusion protein comprising a secretion signal and a phosphatidylserine (PS) binding domain operably linked to an immunostimulatory domain, wherein the immunostimulatory domain further comprise a conjugated small molecule.

Disclosed herein is an isolated nucleic acid molecule comprising a nucleic acid sequence encoding a chimeric fusion protein comprising a PS-binding domain operably linked to an IgG1 Fc domain. Disclosed herein is an isolated nucleic acid molecule comprising a nucleic acid sequence encoding a chimeric fusion protein comprising a PS-binding domain operably linked to an IgG1 Fc domain and an additional immunostimulatory domain. Disclosed herein is an isolated nucleic acid molecule comprising a nucleic acid sequence encoding a chimeric fusion protein comprising a PS-binding domain operably linked to an IgG1 Fc domain and an additional immunostimulatory domain, wherein the additional immunostimulatory domain comprises the IgV domain of SIRPα or the extracellular domain of NKG2D protein.

Disclosed herein is an isolated nucleic acid molecule comprising a nucleic acid sequence encoding a chimeric fusion protein comprising a PS-binding domain operably linked to a single-chain antibody (scFV) to a human CD3 protein. Disclosed herein is an isolated nucleic acid molecule comprising a nucleic acid sequence encoding a chimeric fusion protein comprising an Annexin-binding domain operably linked to a single-chain antibody (scFV) to a human CD3 protein. Disclosed herein is an isolated nucleic acid molecule comprising a nucleic acid sequence encoding a single-chain antibody (scFV) to a human CD3 protein and one or more additional immunostimulatory domains. Disclosed herein is an isolated nucleic acid molecule comprising a nucleic acid sequence encoding a chimeric fusion protein comprising an Annexin-binding domain operably linked to a single-chain antibody (scFV) to a human CD3 protein and one or more additional immunostimulatory domains.

Disclosed herein is an isolated nucleic acid molecule comprising a nucleic acid sequence encoding a chimeric fusion protein comprising the sequence set forth in SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:99, SEQ ID NO:100, SEQ ID NO:101, SEQ ID NO:106, or a fragment thereof.

In an aspect, a disclosed isolated nucleic acid molecule can comprise a nucleic acid sequence encoding a disclosed chimeric fusion protein comprising the structure set forth in FIG. 5A, FIG. 5B, FIG. 5C, FIG. 5D, FIG. 5E, FIG. 5F, or FIG. 5G. In an aspect, a discloses isolated nucleic acid molecule can comprise the sequence encoding a disclosed bi-specific chimeric fusion protein (e.g., PS binding domain and anti-CD3). In an aspect, a disclosed isolated nucleic acid molecule can comprises a nucleic acid sequence encoding a disclosed chimeric fusion protein can comprise the structure set forth in FIG. 5A, FIG. 5B, FIG. 5C, FIG. 5D, FIG. 5E, FIG. 5F, or FIG. 5G, wherein the PS-binding domain comprises any disclosed PS-binding domain and wherein the anti-CD3 binding domain comprises any disclosed binding domain. In an aspect, a disclosed bi-specific chimeric fusion protein can comprise a disclosed PS-binding domain and an anti-CD marker for hematological cancer (e.g., CD1a, CD2, CD3, CD4, CD5, CD7, CD8, CD9, CD10, CD11b, CD11c, CD13, CD14, CD15, CD16, CD19, CD20, CD22, CD23, CD25, CD26, CD30, CD33, CD34, CD36, CD38, CD41, CD43, CD45, CD45RA, CD45RO, CD52, CD56, CD58, CD61, CD64, CD71, CD79a, CD79b, CD103, CD117, CD123, CD138, or CD163).

In an aspect, a disclosed nucleic sequence can be a DNA sequence or an RNA sequence.

In an aspect of a disclosed isolated nucleic acid molecule encoding a chimeric fusion protein, a disclosed PS binding domain can comprise Annexin A1 (ANXA1) or the PS-binding core domain, Annexin A2 (ANXA1), Annexin A3 (ANXA1), Annexin A4 (ANXA1), Annexin A5 (ANXA1), Annexin A6 (ANXA1), Annexin A7 (ANXA1), Annexin A8 (ANXA1), Annexin A8 Like 1 (ANXA1), Annexin A9 (ANXA1), Annexin A10 (ANXA1), Annexin A11 (ANXA1), Annexin A13 (ANXA1), Adhesion G Protein Coupled Receptor B1 (ADGRB1) or the extracellular domain thereof, Apolipoprotein H (APO-H), Coagulation Factor II (F2), Coagulation Factor VII (F7), Coagulation Factor IX (F9), Coagulation Factor X (F10), Growth Arrest Specific 6 (CAS6), Milk Fat Globule EGF And Factor V/VIII Domain Containing (MFGE8), Advanced Glycosylation End-Product Specific Receptor (AGER) or the extracellular domain thereof, Jumonji Domain-Containing Protein 6 (JMJD6); Protein S (PROS1), Hepatitis A Virus Cellular Receptor 1 (HAVCR1) or the extracellular domain thereof, Hepatitis A Virus Cellular Receptor 2 (HAVCR2) or the extracellular domain thereof, T Cell Immunoglobulin and Mucin Domain Containing (TIM-3) or the extracellular domain thereof, Protein Kinase C Alpha (PRKCA) or the C2 domain thereof, Synaptotagmin (SYT1) or the C2A domain thereof, Stabilin-1 (STAB1) or the extracellular domain thereof, Stabilin-2 (STAB2) or the extracellular domain thereof, or any combination thereof.

In an aspect, a disclosed ANXA1 can comprise the amino acid sequence set forth in SEQ ID NO:01 or a fragment thereof. In an aspect, a disclosed ANXA1 can comprise a sequence having at least 70%, at least 75%, at least 80%, at least 85%, at least 90%, or at least 95% identity to the sequence set forth in SEQ ID NO:01 or a fragment thereof. In an aspect, a disclosed ANXA1 can comprise the amino acid sequence set forth in SEQ ID NO:02 or a fragment thereof. In an aspect, a disclosed ANXA1 can comprise a sequence having at least 70%, at least 75%, at least 80%, at least 85%, at least 90%, or at least 95% identity to the sequence set forth in SEQ ID NO:02 or a fragment thereof. In an aspect, a disclosed ANXA2 can comprise the amino acid sequence set forth in SEQ ID NO:03 or a fragment thereof. In an aspect, a disclosed ANXA2 can comprise a sequence having at least 70%, at least 75%, at least 80%, at least 85%, at least 90%, or at least 95% identity to the sequence set forth in SEQ ID NO:03 or a fragment thereof. In an aspect, a disclosed ANXA3 can comprise the amino acid sequence set forth in SEQ ID NO:04 or a fragment thereof. In an aspect, a disclosed ANXA3 can comprise a sequence having at least 70%, at least 75%, at least 80%, at least 85%, at least 90%, or at least 95% identity to the sequence set forth in SEQ ID NO:04 or a fragment thereof. In an aspect, a disclosed ANXA4 can comprise the amino acid sequence set forth in SEQ ID NO:05 or a fragment thereof. In an aspect, a disclosed ANXA4 can comprise a sequence having at least 70%, at least 75%, at least 80%, at least 85%, at least 90%, or at least 95% identity to the sequence set forth in SEQ ID NO:05 or a fragment thereof. In an aspect, a disclosed ANXA5 can comprise the amino acid sequence set forth in SEQ ID NO:06 or a fragment thereof. In an aspect, a disclosed ANXA5 can comprise a sequence having at least 70%, at least 75%, at least 80%, at least 85%, at least 90%, or at least 95% identity to the sequence set forth in SEQ ID NO:06 or a fragment thereof. In an aspect, a disclosed ANXA6 can comprise the amino acid sequence set forth in SEQ ID NO:07 or a fragment thereof. In an aspect, a disclosed ANXA6 can comprise a sequence having at least 70%, at least 75%, at least 80%, at least 85%, at least 90%, or at least 95% identity to the sequence set forth in SEQ ID NO:07 or a fragment thereof. In an aspect, a disclosed ANXA7 can comprise the amino acid sequence set forth in SEQ ID NO:08 or a fragment thereof. In an aspect, a disclosed ANXA7 can comprise a sequence having at least 70%, at least 75%, at least 80%, at least 85%, at least 90%, or at least 95% identity to the sequence set forth in SEQ ID NO:08 or a fragment thereof. In an aspect, a disclosed ANXA8 can comprise the amino acid sequence set forth in SEQ ID NO:09 or a fragment thereof. In an aspect, a disclosed ANXA8 can comprise a sequence having at least 70%, at least 75%, at least 80%, at least 85%, at least 90%, or at least 95% identity to the sequence set forth in SEQ ID NO:09 or a fragment thereof. In an aspect, a disclosed ANXA8L1 can comprise the amino acid sequence set forth in SEQ ID NO:10 or a fragment thereof. In an aspect, a disclosed ANXA8L1 can comprise a sequence having at least 70%, at least 75%, at least 80%, at least 85%, at least 90%, or at least 95% identity to the sequence set forth in SEQ ID NO:10 or a fragment thereof. In an aspect, a disclosed ANXA9 can comprise the amino acid sequence set forth in SEQ ID NO:11 or a fragment thereof. In an aspect, a disclosed ANXA9 signal peptide can comprise a sequence having at least 70%, at least 75%, at least 80%, at least 85%, at least 90%, or at least 95% identity to the sequence set forth in SEQ ID NO:11 or a fragment thereof. In an aspect, a disclosed ANXA10 can comprise the amino acid sequence set forth in SEQ ID NO:12 or a fragment thereof. In an aspect, a disclosed ANXA10 can comprise a sequence having at least 70%, at least 75%, at least 80%, at least 85%, at least 90%, or at least 95% identity to the sequence set forth in SEQ ID NO:12 or a fragment thereof. In an aspect, a disclosed ANXA11 can comprise the amino acid sequence set forth in SEQ ID NO:13 or a fragment thereof. In an aspect, a disclosed ANXA11 can comprise a sequence having at least 70%, at least 75%, at least 80%, at least 85%, at least 90%, or at least 95% identity to the sequence set forth in SEQ ID NO:13 or a fragment thereof. In an aspect, a disclosed ANXA13 can comprise the amino acid sequence set forth in SEQ ID NO:14 or a fragment thereof. In an aspect, a disclosed ANXA13 can comprise a sequence having at least 70%, at least 75%, at least 80%, at least 85%, at least 90%, or at least 95% identity to the sequence set forth in SEQ ID NO:14 or a fragment thereof.

In an aspect, a disclosed ADGRB1 can comprise the amino acid sequence set forth in SEQ ID NO:15 or a fragment thereof. In an aspect, a disclosed ADGRB1 can comprise a sequence having at least 70%, at least 75%, at least 80%, at least 85%, at least 90%, or at least 95% identity to the sequence set forth in SEQ ID NO:15 or a fragment thereof. In an aspect, a disclosed ADGRB1 can comprise the amino acid sequence set forth in SEQ ID NO:16 or a fragment thereof. In an aspect, a disclosed ADGRB1 can comprise a sequence having at least 70%, at least 75%, at least 80%, at least 85%, at least 90%, or at least 95% identity to the sequence set forth in SEQ ID NO:16 or a fragment thereof. In an aspect, a disclosed APO-H can comprise the amino acid sequence set forth in SEQ ID NO:17 or a fragment thereof. In an aspect, a disclosed APO-H can comprise a sequence having at least 70%, at least 75%, at least 80%, at least 85%, at least 90%, or at least 95% identity to the sequence set forth in SEQ ID NO:17 or a fragment thereof. In an aspect, a disclosed F2 can comprise the amino acid sequence set forth in SEQ ID NO:18 or a fragment thereof. In an aspect, a disclosed F2 can comprise a sequence having at least 70%, at least 75%, at least 80%, at least 85%, at least 90%, or at least 95% identity to the sequence set forth in SEQ ID NO:18 or a fragment thereof. In an aspect, a disclosed F7 can comprise the amino acid sequence set forth in SEQ ID NO:19 or a fragment thereof. In an aspect, a disclosed F7 can comprise a sequence having at least 70%, at least 75%, at least 80%, at least 85%, at least 90%, or at least 95% identity to the sequence set forth in SEQ ID NO:19 or a fragment thereof. In an aspect, a disclosed F9 can comprise the amino acid sequence set forth in SEQ ID NO:20 or a fragment thereof. In an aspect, a disclosed F9 can comprise a sequence having at least 70%, at least 75%, at least 80%, at least 85%, at least 90%, or at least 95% identity to the sequence set forth in SEQ ID NO:20 or a fragment thereof. In an aspect, a disclosed F10 can comprise the amino acid sequence set forth in SEQ ID NO:21 or a fragment thereof. In an aspect, a disclosed F10 can comprise a sequence having at least 70%, at least 75%, at least 80%, at least 85%, at least 90%, or at least 95% identity to the sequence set forth in SEQ ID NO:21 or a fragment thereof. In an aspect, a disclosed GAS6 can comprise the amino acid sequence set forth in SEQ ID NO:22 or a fragment thereof. In an aspect, a disclosed GAS6 an comprise a sequence having at least 70%, at least 75%, at least 80%, at least 85%, at least 90%, or at least 95% identity to the sequence set forth in SEQ ID NO:22 or a fragment thereof. In an aspect, a disclosed MFGE8 can comprise the amino acid sequence set forth in SEQ ID NO:23 or a fragment thereof. In an aspect, a disclosed MFGE8 can comprise a sequence having at least 70%, at least 75%, at least 80%, at least 85%, at least 90%, or at least 95% identity to the sequence set forth in SEQ ID NO:23 or a fragment thereof. In an aspect, a disclosed AGER can comprise the amino acid sequence set forth in SEQ ID NO:24 or a fragment thereof. In an aspect, a disclosed AGER can comprise a sequence having at least 70%, at least 75%, at least 80%, at least 85%, at least 90%, or at least 95% identity to the sequence set forth in SEQ ID NO:24 or a fragment thereof. In an aspect, a disclosed AGER can comprise the amino acid sequence set forth in SEQ ID NO:25 or a fragment thereof. In an aspect, a disclosed AGER can comprise a sequence having at least 70%, at least 75%, at least 80%, at least 85%, at least 90%, or at least 95% identity to the sequence set forth in SEQ ID NO:25 or a fragment thereof. In an aspect, a disclosed PROS1 can comprise the amino acid sequence set forth in SEQ ID NO:26 or a fragment thereof. In an aspect, a disclosed PROS1 can comprise a sequence having at least 70%, at least 75%, at least 80%, at least 85%, at least 90%, or at least 95% identity to the sequence set forth in SEQ ID NO:26 or a fragment thereof. In an aspect, a disclosed STAB1 can comprise the amino acid sequence set forth in SEQ ID NO:27 or a fragment thereof. In an aspect, a disclosed STAB1 can comprise a sequence having at least 70%, at least 75%, at least 80%, at least 85%, at least 90%, or at least 95% identity to the sequence set forth in SEQ ID NO:27 or a fragment thereof. In an aspect, a disclosed STAB1 can comprise the amino acid sequence set forth in SEQ ID NO:28 or a fragment thereof. In an aspect, a disclosed STAB1 can comprise a sequence having at least 70%, at least 75%, at least 80%, at least 85%, at least 90%, or at least 95% identity to the sequence set forth in SEQ ID NO:28 or a fragment thereof. In an aspect, a disclosed STAB2 can comprise the amino acid sequence set forth in SEQ ID NO:29 or a fragment thereof. In an aspect, a disclosed STAB2 can comprise a sequence having at least 70%, at least 75%, at least 80%, at least 85%, at least 90%, or at least 95% identity to the sequence set forth in SEQ ID NO:29 or a fragment thereof. In an aspect, a disclosed STAB2 can comprise the amino acid sequence set forth in SEQ ID NO:30 or a fragment thereof. In an aspect, a disclosed STAB2 can comprise a sequence having at least 70%, at least 75%, at least 80%, at least 85%, at least 90%, or at least 95% identity to the sequence set forth in SEQ ID NO:30 or a fragment thereof.

In an aspect, a disclosed HAVCR1 can comprise the amino acid sequence set forth in SEQ ID NO:31 or a fragment thereof. In an aspect, a disclosed HAVCR1 can comprise a sequence having at least 70%, at least 75%, at least 80%, at least 85%, at least 90%, or at least 95% identity to the sequence set forth in SEQ ID NO:31 or a fragment thereof. In an aspect, a disclosed HAVCR1 can comprise the amino acid sequence set forth in SEQ ID NO:32 or a fragment thereof. In an aspect, a disclosed HAVCR1 can comprise a sequence having at least 70%, at least 75%, at least 80%, at least 85%, at least 90%, or at least 95% identity to the sequence set forth in SEQ ID NO:32 or a fragment thereof. In an aspect, a disclosed HAVCR2 can comprise the amino acid sequence set forth in SEQ ID NO:33 or a fragment thereof. In an aspect, a disclosed HAVCR2 can comprise a sequence having at least 70%, at least 75%, at least 80%, at least 85%, at least 90%, or at least 95% identity to the sequence set forth in SEQ ID NO:33 or a fragment thereof. In an aspect, a disclosed HAVCR2 can comprise the amino acid sequence set forth in SEQ ID NO:34 or a fragment thereof. In an aspect, a disclosed HAVCR2 can comprise a sequence having at least 70%, at least 75%, at least 80%, at least 85%, at least 90%, or at least 95% identity to the sequence set forth in SEQ ID NO:34 or a fragment thereof. In an aspect, a disclosed TIMD4 can comprise the amino acid sequence set forth in SEQ ID NO:35 or a fragment thereof. In an aspect, a disclosed TIMD4 can comprise a sequence having at least 70%, at least 75%, at least 80%, at least 85%, at least 90%, or at least 95% identity to the sequence set forth in SEQ ID NO:35 or a fragment thereof. In an aspect, a disclosed TIMD4 can comprise the amino acid sequence set forth in SEQ ID NO:36 or a fragment thereof. In an aspect, a disclosed TIMD4 can comprise a sequence having at least 70%, at least 75%, at least 80%, at least 85%, at least 90%, or at least 95% identity to the sequence set forth in SEQ ID NO:36 or a fragment thereof. In an aspect, a disclosed PRKCA can comprise the amino acid sequence set forth in SEQ ID NO:37 or a fragment thereof. In an aspect, a disclosed PRKCA can comprise a sequence having at least 70%, at least 75%, at least 80%, at least 85%, at least 90%, or at least 95% identity to the sequence set forth in SEQ ID NO:37 or a fragment thereof. In an aspect, a disclosed PRKCA can comprise the amino acid sequence set forth in SEQ ID NO:38 or a fragment thereof. In an aspect, a disclosed PRKCA can comprise a sequence having at least 70%, at least 75%, at least 80%, at least 85%, at least 90%, or at least 95% identity to the sequence set forth in SEQ ID NO:38 or a fragment thereof. In an aspect, a disclosed SYT1 can comprise the amino acid sequence set forth in SEQ ID NO:39 or a fragment thereof. In an aspect, a disclosed SYT1 can comprise a sequence having at least 70%, at least 75%, at least 80%, at least 85%, at least 90%, or at least 95% identity to the sequence set forth in SEQ ID NO:39 or a fragment thereof. In an aspect, a disclosed SYT1 can comprise the amino acid sequence set forth in SEQ ID NO:40 or a fragment thereof. In an aspect, a disclosed SYT1 can comprise a sequence having at least 70%, at least 75%, at least 80%, at least 85%, at least 90%, or at least 95% identity to the sequence set forth in SEQ ID NO:40 or a fragment thereof. In an aspect, a disclosed JMJD6 can comprise the amino acid sequence set forth in SEQ ID NO:41 or a fragment thereof. In an aspect, a disclosed JMJD6 can comprise a sequence having at least 70%, at least 75%, at least 80%, at least 85%, at least 90%, or at least 95% identity to the sequence set forth in SEQ ID NO:41 or a fragment thereof.

In an aspect, a disclosed KLRK1 can comprise the amino acid sequence set forth in SEQ ID NO:102 or a fragment thereof. In an aspect, a disclosed KLRK1 can comprise a sequence having at least 70%, at least 75%, at least 80%, at least 85%, at least 90%, or at least 95% identity to the sequence set forth in SEQ ID NO:102 or a fragment thereof. In an aspect, a disclosed KLRK1 can comprise the amino acid sequence set forth in SEQ ID NO:103 or a fragment thereof. In an aspect, a disclosed KLRK1 can comprise a sequence having at least 70%, at least 75%, at least 80%, at least 85%, at least 90%, or at least 95% identity to the sequence set forth in SEQ ID NO:103 or a fragment thereof.

In an aspect, a disclosed secretion signal can be any secretion signal known to the art. In an aspect, a disclosed secretion signal can comprise the sequence set forth in SEQ ID NO:107-SEQ ID NO:108.

In an aspect, a disclosed nucleic acid sequence can be an RNA sequence or a mRNA sequence. In an aspect, a disclosed mRNA sequence can be transcribed into an encoded PS binding domain. In an aspect, a disclosed mRNA sequence can be transcribed into Annexin A1 (ANXA1) or the PS-binding core domain, Annexin A2 (ANXA1), Annexin A3 (ANXA1), Annexin A4 (ANXA1), Annexin A5 (ANXA1), Annexin A6 (ANXA1), Annexin A7 (ANXA1), Annexin A8 (ANXA1), Annexin A8 Like 1 (ANXA1), Annexin A9 (ANXA1), Annexin A10 (ANXA1), Annexin A11 (ANXA1), Annexin A13 (ANXA1), Adhesion G Protein Coupled Receptor B1 (ADGRB1) or the extracellular domain thereof, Apolipoprotein H (APO-H), Coagulation Factor II (F2), Coagulation Factor VII (F7), Coagulation Factor IX (F9), Coagulation Factor X (F10), Growth Arrest Specific 6 (CAS6), Milk Fat Globule EGF And Factor V/VIII Domain Containing (MFGE8), Advanced Glycosylation End-Product Specific Receptor (AGER) or the extracellular domain thereof, Jumonji Domain-Containing Protein 6 (JMJD6); Protein S (PROS1), Hepatitis A Virus Cellular Receptor 1 (HAVCR1) or the extracellular domain thereof, Hepatitis A Virus Cellular Receptor 2 (HAVCR2) or the extracellular domain thereof, T Cell Immunoglobulin and Mucin Domain Containing (TIM-3) or the extracellular domain thereof, Protein Kinase C Alpha (PRKCA) or the C2 domain thereof, Synaptotagmin (SYT1) or the C2A domain thereof, Stabilin-1 (STAB1) or the extracellular domain thereof, Stabilin-2 (STAB2) or the extracellular domain thereof, or any combination thereof.

In an aspect, a disclosed mRNA sequence can be transcribed into Annexin A1 (ANXA1) or the PS-binding core domain, Annexin A2 (ANXA1), Annexin A3 (ANXA1), Annexin A4 (ANXA1), Annexin A5 (ANXA1), Annexin A6 (ANXA1), Annexin A7 (ANXA1), Annexin A8 (ANXA1), Annexin A8 Like 1 (ANXA1), Annexin A9 (ANXA1), Annexin A10 (ANXA1), Annexin A11 (ANXA1), or Annexin A13 (ANXA1) can comprise the sequence set forth in SEQ ID NO:56-SEQ ID NO:83.

In an aspect, a disclosed mRNA sequence can be transcribed into Annexin A1 (ANXA1) or the PS-binding core domain, Annexin A2 (ANXA1), Annexin A3 (ANXA1), Annexin A4 (ANXA1), Annexin A5 (ANXA1), Annexin A6 (ANXA1), Annexin A7 (ANXA1), Annexin A8 (ANXA1), Annexin A8 Like 1 (ANXA1), Annexin A9 (ANXA1), Annexin A10 (ANXA1), Annexin A11 (ANXA1), or Annexin A13 (ANXA1) can comprise a sequence having at least 70%, at least 75%, at least 80%, at least 85%, at least 90%, or at least 95% identity to the sequence set forth in SEQ ID NO:56-SEQ ID NO:83.

In an aspect of a disclosed isolated nucleic acid molecule, a disclosed encoded PS binding domain can comprise the single-chain variable domain of bavituximab. In an aspect, a disclosed encoded PS binding domain can comprise the sequence set forth in SEQ ID NO:55 or a fragment thereof. In an aspect, a disclosed encoded PS binding chain can comprise a sequence having at least 70%, at least 75%, at least 80%, at least 85%, at least 90%, or at least 95% identity to the sequence set forth in SEQ ID NO:55 or a fragment thereof. In an aspect, a disclosed encoded PS binding domain can comprise the single-chain variable domain of PGN632. In an aspect, a disclosed PS binding domain can comprise the single-chain variable domain of PGN632. In an aspect, a single-chain variable domain of PGN632 can comprise a γ1 heavy chain and a λ light chain. In an aspect, a single-chain variable domain of PGN632 can bind to cardiolipin/PS. In an aspect, a disclosed PS binding domain can comprise the single-chain variable domain of P1. In an aspect, a single-chain variable domain of P1 can comprise a γ1 heavy chain and a λ light chain. In an aspect, a single-chain variable domain of P1 can bind to cardiolipin/PS. In an aspect, a disclosed PS binding domain can comprise the single-chain variable domain of IS4. In an aspect, a single-chain variable domain of IS4 can comprise a γ3 VH1 heavy chain and a λ Vλ2 light chain. In an aspect, a single-chain variable domain of IS4 can bind to cardiolipin/PS. In an aspect, a disclosed PS binding domain can comprise the single-chain variable domain of CL1. In an aspect, a single-chain variable domain of CLL can comprise a γ3 VH1 heavy chain and a λ Vλ3 light chain. In an aspect, a single-chain variable domain of IS4 can bind to cardiolipin/PS. In an aspect, PGN632, P1, IS4, and CLL are described in Moody et al. (2010) J. Exp. Med. 207(4):763-776, which is incorporated herein by reference for its teachings of these antibodies and their characteristics.

In an aspect of a disclosed isolated nucleic acid molecule, a disclosed encoded PS binding domain can comprise a scFV and wherein the scFV can comprise a linker. In an aspect, a disclosed encoded linker can join the V_{H} and V_{L} regions of the ScFv.

In an aspect, a disclosed isolated nucleic acid molecule can be introduced to T cells and/or NK cells. In an aspect, a disclosed isolated nucleic acid molecule can be used to activated one or more types of immune cells (e.g., naive T cells, central memory T cells, effector memory T cells, NK cells or combination thereof) upon antigen binding.

In an aspect, a disclosed isolated nucleic acid molecule can stimulate an effector cell mediated immune modulator response to PS-expressing tumor cells. In an aspect, a disclosed isolated nucleic acid molecule can induce a tumor reducing immune response. In an aspect, a disclosed isolated nucleic acid molecule can induce phagocytosis of cancer cells in the subject. In an aspect, a disclosed isolated nucleic acid molecule can cross-prime an anti-tumor T cell response. In an aspect, a disclosed isolated nucleic acid molecule can induce a tumor eliminating immune response. In an aspect, a disclosed isolated nucleic acid molecule can effect cell death of PS-expression cells. In an aspect, a disclosed isolated nucleic acid molecule can treat cancer.

In an aspect, a disclosed isolated nucleic acid molecule can induce cell death in the targeted cell. In an aspect, a disclosed isolated nucleic acid molecule can stimulate an effector cell mediated immune modulator response to PS-expressing tumor cells. In an aspect, a disclosed isolated nucleic acid molecule can induce a tumor reducing immune response. In an aspect, a disclosed isolated nucleic acid molecule can induce phagocytosis of cancer cells in the subject. In an aspect, a disclosed isolated nucleic acid molecule can cross-prime an anti-tumor T cell response. In an aspect, a disclosed isolated nucleic acid molecule can induce a tumor eliminating immune response. In an aspect, a disclosed ADC can treat cancer. In an aspect, a disclosed isolated nucleic acid molecule can improve and/or extend the survivability of the subject, can improve a subject's quality of life, can increase and/or prolong a subject's life span, or any combination thereof.

### 4. Vectors

Disclosed herein is a vector comprising a disclosed isolated nucleic acid molecule. Disclosed herein is a vector comprising an isolated nucleic acid molecule comprising a nucleic acid sequence encoding one or more disclosed chimeric fusion proteins. Disclosed herein is a vector comprising an isolated nucleic acid molecule comprising a nucleic acid sequence encoding a chimeric fusion protein comprising a phosphatidylserine (PS) binding domain. Disclosed herein is a vector comprising an isolated nucleic acid molecule comprising a nucleic acid sequence encoding a chimeric fusion protein comprising a phosphatidylserine (PS) binding domain and an immunostimulatory domain. Disclosed herein is a vector comprising an isolated nucleic acid molecule comprising a nucleic acid sequence encoding a chimeric fusion protein comprising a phosphatidylserine (PS) binding domain operably linked an immunostimulatory domain. Disclosed herein is a vector comprising an isolated nucleic acid molecule comprising a nucleic acid sequence encoding a phosphatidylserine (PS) binding domain, a linker, and an immunostimulatory domain. Disclosed herein is a vector comprising an isolated nucleic acid molecule comprising a nucleic acid sequence encoding a chimeric fusion protein comprising a phosphatidylserine (PS) binding domain operably linked to an immunostimulatory domain, wherein the immunostimulatory domain further comprise a conjugated small molecule. Disclosed herein is a vector comprising an isolated nucleic acid molecule comprising a nucleic acid sequence encoding a chimeric fusion protein comprising a secretion signal and a phosphatidylserine (PS) binding domain.

Disclosed herein is a vector comprising an isolated nucleic acid molecule comprising a nucleic acid sequence encoding a chimeric fusion protein comprising a secretion signal, a phosphatidylserine (PS) binding domain, and an immunostimulatory domain. Disclosed herein is a vector comprising an isolated nucleic acid molecule comprising a nucleic acid sequence encoding a chimeric fusion protein comprising a secretion signal and a phosphatidylserine (PS) binding domain operably linked an immunostimulatory domain. Disclosed herein is a vector comprising an isolated nucleic acid molecule comprising a nucleic acid sequence encoding a chimeric fusion protein comprising a secretion signal, a phosphatidylserine (PS) binding domain, a linker, and an immunostimulatory domain. Disclosed herein is a vector comprising an isolated nucleic acid molecule comprising a nucleic acid sequence encoding a chimeric fusion protein comprising a secretion signal and a phosphatidylserine (PS) binding domain operably linked to an immunostimulatory domain, wherein the immunostimulatory domain further comprise a conjugated small molecule. Disclosed herein is a vector comprising an isolated nucleic acid molecule comprising a nucleic acid sequence encoding a chimeric fusion protein comprising a PS-binding domain operably linked to an IgG1 Fc domain. Disclosed herein is a vector comprising an isolated nucleic acid molecule comprising a nucleic acid sequence encoding a chimeric fusion protein comprising a PS-binding domain operably linked to an IgG1 Fc domain and an additional immunostimulatory domain. Disclosed herein is a vector comprising an isolated nucleic acid molecule comprising a nucleic acid sequence encoding a chimeric fusion protein comprising a PS-binding domain operably linked to an IgG1 Fc domain and an additional immunostimulatory domain, wherein the additional immunostimulatory domain comprises the IgV domain of SIRPα or the extracellular domain of NKG2D protein.

Disclosed herein is a vector comprising an isolated nucleic acid molecule comprising a nucleic acid sequence encoding a chimeric fusion protein comprising a PS-binding domain operably linked to a single-chain antibody (scFV) to a human CD3 protein. Disclosed herein is a vector comprising an isolated nucleic acid molecule comprising a nucleic acid sequence encoding a chimeric fusion protein comprising an Annexin-binding domain operably linked to a single-chain antibody (scFV) to a human CD3 protein. Disclosed herein is a vector comprising an isolated nucleic acid molecule comprising a nucleic acid sequence encoding a single-chain antibody (scFV) to a human CD3 protein and one or more additional immunostimulatory domains.

Disclosed herein is a vector comprising an isolated nucleic acid molecule comprising a nucleic acid sequence encoding a chimeric fusion protein comprising an Annexin-binding domain operably linked to a single-chain antibody (scFV) to a human CD3 protein and one or more additional immunostimulatory domains.

Disclosed herein is a vector comprising an isolated nucleic acid molecule comprising a nucleic acid sequence encoding a chimeric fusion protein comprising the sequence set forth in SEQ ID NO: SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:99, SEQ ID NO:100, SEQ ID NO:101, SEQ ID NO:106, or a fragment thereof.

In an aspect, a disclosed vector can comprise a disclosed isolated nucleic acid molecule encoding a disclosed chimeric fusion protein having a structure set forth in FIG. 5A, FIG. 5B, FIG. 5C, FIG. 5D, FIG. 5E, FIG. 5F, or FIG. 5G. In an aspect, a disclosed chimeric fusion protein is bi-specific (e.g., PS binding domain and anti-CD3). In an aspect, a disclosed vector can comprise a disclosed isolated nucleic acid molecule encoding a disclosed chimeric fusion protein having a structure set forth in FIG. 5A, FIG. 5B, FIG. 5C, FIG. 5D, FIG. 5E, FIG. 5F, or FIG. 5G, wherein the PS-binding domain comprises any disclosed PS-binding domain and wherein the anti-CD3 binding domain comprises any disclosed binding domain. In an aspect, a disclosed bi-specific chimeric fusion protein can comprise a disclosed PS-binding domain and an anti-CD marker for hematological cancer (e.g., CD1a, CD2, CD3, CD4, CD5, CD7, CD8, CD9, CD10, CD11b, CD11c, CD13, CD14, CD15, CD16, CD19, CD20, CD22, CD23, CD25, CD26, CD30, CD33, CD34, CD36, CD38, CD41, CD43, CD45, CD45RA, CD45RO, CD52, CD56, CD58, CD61, CD64, CD71, CD79a, CD79b, CD103, CD117, CD123, CD138, or CD163).

In an aspect, a disclosed nucleic sequence can be a DNA sequence or an RNA sequence.

In an aspect, a disclosed vector can be used to introduce a disclosed isolated nucleic acid molecule or disclosed isolated nucleic acid sequence to one or more host cells. In an aspect, host cells are discussed infra. In an aspect, a disclosed cell can comprise T cells, NK cells, macrophages, or iPSCs. In an aspect, a disclosed cell can comprise any cell capable of generating a disclosed chimeric fusion protein.

In an aspect, a disclosed vector can be used to introduce a disclosed isolated nucleic acid molecule encoding of a disclosed chimeric fusion protein to one or more host cells. In an aspect, a disclosed vector can be used to introduce a disclosed isolated nucleic acid molecule encoding of a disclosed chimeric fusion protein to one or more T cells or NK cells or macrophages.

In an aspect, a disclosed vector can be an integrating vector or a non-integrating vector. In an aspect, integration can mean that the nucleotides of nucleic acid sequence can be stably inserted into the cellular genome (e.g., covalently linked to the nucleic acid sequence within the cell's chromosomal DNA).

In an aspect, a disclosed vector can be a viral vector or a non-viral vector. In an aspect, a disclosed non-viral vector can be a polymer-based vector, a peptide-based vector, a lipid nanoparticle, a solid lipid nanoparticle, or a cationic lipid-based vector.

In an aspect, a disclosed vector can be a transposon-based vector such as Sleeping Beauty and PiggyBac, both of which are known in the art. In an aspect, a first plasmid can be loaded with a disclosed nucleic acid sequence encoding a disclosed chimeric fusion protein, named transposon, surrounded by inverted repeats (IRs) that contain short direct repeats (DRs), while a second plasmid encodes the enzyme (transposase) that can recognize the sequences from the first plasmid and cut the transposon out of the first plasmid. Then the disclosed sequence encoded a disclosed chimeric fusion protein can be successfully delivered into the targeted cell (e.g., a T cell or a NK cell or a macrophage) cytoplasm and inserted randomly into TA dinucleotide base pairs of the recipient DNA sequence.

In an aspect, a disclosed vector can comprise lipid and/or polymer-based nanoparticles loaded with mRNA encoding a disclosed chimeric fusion protein. In an aspect, a disclosed vector can comprise lipid and/or polymer-based nanoparticles loaded with transposon-based plasmids such as, for example, transposon-based plasmids comprising a sequence encoding a disclosed chimeric fusion protein. In an aspect, a disclosed vector can comprise mRNA encoding a disclosed chimeric fusion protein. In an aspect, a disclosed vector can comprise lipid and/or polymer-based nanoparticles loaded with mRNA encoding a disclosed chimeric fusion protein.

In an aspect, a disclosed viral vector can be an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector. In an aspect, a disclosed viral vector can be an adeno-associated virus (AAV) vector. In an aspect, a disclosed AAV vector can include naturally isolated serotypes including, but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, an AAV capsid can be a chimera either created by capsid evolution or by rational capsid engineering from a naturally isolated AAV variants to capture desirable serotype features such as enhanced or specific tissue tropism and/or a host immune response escape. Naturally isolated AAV variants include, but not limited to, AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, a disclosed AAV vector can be AAV-Rh74 or a related variant (e.g., capsid variants like RHM4-1). In an aspect, a disclosed AAV vector can be a self-complementary AAV as disclosed herein.

In an aspect, a disclosed vector can be a recombinant vector comprising a disclosed nucleic acid sequence. Recombinant vectors (such as recombinant viral vectors) are known to the art.

In an aspect, a disclosed promoter can comprise a ubiquitous promoter, a constitutive promoter, or a tissue specific promoter. In an aspect, a disclosed promoter can be operably linked to a disclosed nucleic acid sequence encoding a disclosed chimeric fusion protein. In an aspect, a disclosed promoter can be operably linked to a disclosed nucleic acid sequence encoding a disclosed tumor antigen. Promoters are known to the art. In an aspect, a disclosed promoter can be a promoter/enhancer. Promoter/enhancers are known to the art. In an aspect, a disclosed promoter can be an endogenous promoter. In an aspect, a disclosed endogenous promoter can be an endogenous promoter/enhancer. In an aspect, a disclosed promoter or a disclosed promoter/enhancer can be used for constitutive and efficient expression of a disclosed chimeric fusion protein. In an aspect, a disclosed promoter or a disclosed promoter/enhancer can be used for constitutive and efficient expression of a disclosed tumor antigen. In an aspect, a disclosed vector can be used to engineer cells to express a disclosed chimeric fusion protein.

In an aspect, a disclosed vector can stimulate an effector cell mediated immune modulator response to PS-expressing tumor cells. In an aspect, a disclosed vector can induce a tumor reducing immune response. In an aspect, a disclosed vector can induce phagocytosis of cancer cells in the subject. In an aspect, a disclosed vector can cross-prime an anti-tumor T cell response. In an aspect, a disclosed vector can induce a tumor eliminating immune response. In an aspect, a disclosed vector can treat cancer.

In an aspect, a disclosed vector can induce cell death in the targeted cell. In an aspect, a disclosed vector can stimulate an effector cell mediated immune modulator response to PS-expressing tumor cells. In an aspect, a disclosed vector can induce a tumor reducing immune response. In an aspect, a disclosed vector can induce phagocytosis of cancer cells in the subject. In an aspect, a disclosed vector can cross-prime an anti-tumor T cell response. In an aspect, a disclosed vector can induce a tumor eliminating immune response. In an aspect, a disclosed ADC can treat cancer. In an aspect, a disclosed vector can improve and/or extend the survivability of the subject, can improve a subject's quality of life, can increase and/or prolong a subject's life span, or any combination thereof.

### 5. Plasmids

Disclosed herein is a plasmid comprising a disclosed isolated nucleic acid molecule. Disclosed herein is a plasmid comprising a disclosed nucleic acid sequence. Disclosed herein is a plasmid comprising an isolated nucleic molecule comprising a nucleic acid sequence encoding one or more disclosed chimeric fusion proteins. Disclosed herein is a plasmid comprising an isolated nucleic acid molecule comprising a nucleic acid sequence encoding a chimeric fusion protein comprising a phosphatidylserine (PS) binding domain. Disclosed herein is a plasmid comprising an isolated nucleic acid molecule comprising a nucleic acid sequence encoding a chimeric fusion protein comprising a phosphatidylserine (PS) binding domain and an immunostimulatory domain.

Disclosed herein is a plasmid comprising an isolated nucleic acid molecule comprising a nucleic acid sequence encoding a chimeric fusion protein comprising a phosphatidylserine (PS) binding domain operably linked an immunostimulatory domain. Disclosed herein is a plasmid comprising an isolated nucleic acid molecule comprising a nucleic acid sequence encoding a phosphatidylserine (PS) binding domain, a linker, and an immunostimulatory domain. Disclosed herein is a plasmid comprising an isolated nucleic acid molecule comprising a nucleic acid sequence encoding a chimeric fusion protein comprising a phosphatidylserine (PS) binding domain operably linked to an immunostimulatory domain, wherein the immunostimulatory domain further comprise a conjugated small molecule. Disclosed herein is a plasmid comprising an isolated nucleic acid molecule comprising a nucleic acid sequence encoding a chimeric fusion protein comprising a secretion signal and a phosphatidylserine (PS) binding domain.

Disclosed herein is a plasmid comprising an isolated nucleic acid molecule comprising a nucleic acid sequence encoding a chimeric fusion protein comprising a secretion signal, a phosphatidylserine (PS) binding domain, and an immunostimulatory domain. Disclosed herein is a plasmid comprising an isolated nucleic acid molecule comprising a nucleic acid sequence encoding a chimeric fusion protein comprising a secretion signal and a phosphatidylserine (PS) binding domain operably linked an immunostimulatory domain. Disclosed herein is a plasmid comprising an isolated nucleic acid molecule comprising a nucleic acid sequence encoding a chimeric fusion protein comprising a secretion signal, a phosphatidylserine (PS) binding domain, a linker, and an immunostimulatory domain. Disclosed herein is a plasmid comprising an isolated nucleic acid molecule comprising a nucleic acid sequence encoding a chimeric fusion protein comprising a secretion signal and a phosphatidylserine (PS) binding domain operably linked to an immunostimulatory domain, wherein the immunostimulatory domain further comprise a conjugated small molecule.

Disclosed herein is a plasmid comprising an isolated nucleic acid molecule comprising a nucleic acid sequence encoding a chimeric fusion protein comprising a PS-binding domain operably linked to an IgG1 Fc domain. Disclosed herein is a plasmid comprising an isolated nucleic acid molecule comprising a nucleic acid sequence encoding a chimeric fusion protein comprising a PS-binding domain operably linked to an IgG1 Fc domain and an additional immunostimulatory domain. Disclosed herein is a plasmid comprising an isolated nucleic acid molecule comprising a nucleic acid sequence encoding a chimeric fusion protein comprising a PS-binding domain operably linked to an IgG1 Fc domain and an additional immunostimulatory domain, wherein the additional immunostimulatory domain comprises the IgV domain of SIRPα or the extracellular domain of NKG2D protein. Disclosed herein is a plasmid comprising an isolated nucleic acid molecule comprising a nucleic acid sequence encoding a chimeric fusion protein comprising a PS-binding domain operably linked to a single-chain antibody (scFV) to a human CD3 protein. Disclosed herein is a plasmid comprising an isolated nucleic acid molecule comprising a nucleic acid sequence encoding a chimeric fusion protein comprising an Annexin-binding domain operably linked to a single-chain antibody (scFV) to a human CD3 protein. Disclosed herein is a plasmid comprising an isolated nucleic acid molecule comprising a nucleic acid sequence encoding a single-chain antibody (scFV) to a human CD3 protein and one or more additional immunostimulatory domains. Disclosed herein is a plasmid comprising an isolated nucleic acid molecule comprising a nucleic acid sequence encoding a chimeric fusion protein comprising an Annexin-binding domain operably linked to a single-chain antibody (scFV) to a human CD3 protein and one or more additional immunostimulatory domains.

Disclosed herein is a plasmid comprising an isolated nucleic acid molecule comprising a nucleic acid sequence encoding a chimeric fusion protein comprising the sequence set forth in SEQ ID NO: SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:99, SEQ ID NO:100, SEQ ID NO:101, SEQ ID NO:106, or a fragment thereof.

Disclosed herein is a plasmid comprising an isolated nucleic acid molecule comprising a nucleic acid sequence encoding a bi-specific chimeric fusion protein having the structure set forth in FIG. 5A, FIG. 5B, FIG. 5C, FIG. 5D, FIG. 5E, FIG. 5F, or FIG. 5G. In an aspect, a disclosed chimeric fusion protein is bi-specific (e.g., PS binding domain and anti-CD3). In an aspect, a disclosed plasmid can comprise an isolated nucleic acid molecule comprising a nucleic acid sequence encoding a disclosed chimeric fusion protein having a structure set forth in FIG. 5A, FIG. 5B, FIG. 5C, FIG. 5D, FIG. 5E, FIG. 5F, or FIG. 5G, wherein the PS-binding domain comprises any disclosed PS-binding domain and wherein the anti-CD3 binding domain comprises any disclosed binding domain. In an aspect, a disclosed bi-specific chimeric fusion protein can comprise a disclosed PS-binding domain and an anti-CD marker for hematological cancer (e.g., CD1a, CD2, CD3, CD4, CD5, CD7, CD8, CD9, CD10, CD11b, CD11c, CD13, CD14, CD15, CD16, CD19, CD20, CD22, CD23, CD25, CD26, CD30, CD33, CD34, CD36, CD38, CD41, CD43, CD45, CD45RA, CD45RO, CD52, CD56, CD58, CD61, CD64, CD71, CD79a, CD79b, CD103, CD117, CD123, CD138, or CD163).

In an aspect, a disclosed plasmid can be used to introduce a disclosed isolated nucleic acid molecule or disclosed isolated nucleic acid sequence to one or more host cells. In an aspect, host cells are discussed infra. In an aspect, a disclosed cell can comprise T cells, NK cells, macrophages, or iPSCs. In an aspect, a disclosed cell can comprise any cell capable of generating a disclosed chimeric fusion protein.

In an aspect, a disclosed plasmid can be used to introduce a disclosed isolated nucleic acid molecule encoding a disclosed chimeric fusion protein or disclosed isolated nucleic acid sequence encoding a disclosed isolated nucleic acid molecule to one or more host cells.

In an aspect, a disclosed plasmid can be used to introduce a disclosed isolated nucleic acid molecule or disclosed isolated nucleic acid sequence to one or more T cells or NK cells or macrophages. In an aspect, a disclosed plasmid can be used to introduce a disclosed isolated nucleic acid molecule or a disclosed isolated nucleic sequence to one or more cells comprise any cell capable of generating a disclosed chimeric fusion protein.

In an aspect, a disclosed plasmid can be used to introduce a disclosed isolated nucleic acid molecule encoding a disclosed isolated nucleic acid molecule or disclosed isolated nucleic acid sequence encoding a disclosed isolated nucleic acid molecule to one or more T cells or NK cells or macrophages. In an aspect, a disclosed plasmid can be used to introduce a disclosed isolated nucleic acid molecule encoding a disclosed isolated nucleic acid molecule or disclosed isolated nucleic acid sequence encoding a disclosed isolated nucleic acid molecule to one or more cells comprise any cell capable of generating a disclosed chimeric fusion protein.

In an aspect, a disclosed plasmid can be an integrating vector or a non-integrating vector. In an aspect, integration can mean that the nucleotides of nucleic acid sequence can be stably inserted into the cellular genome (e.g., covalently linked to the nucleic acid sequence within the cell's chromosomal DNA).

In an aspect, a disclosed plasmid can be a recombinant vector comprising a disclosed nucleic acid sequence. Recombinant plasmids are known to the art.

In an aspect, a disclosed promoter can comprise a ubiquitous promoter, a constitutive promoter, or a tissue specific promoter. In an aspect, a disclosed promoter can be operably linked to a disclosed nucleic acid sequence encoding a disclosed chimeric fusion protein. In an aspect, a disclosed promoter can be operably linked to a disclosed nucleic acid sequence encoding a disclosed tumor antigen. Promoters are known to the art. In an aspect, a disclosed promoter can be a promoter/enhancer. Promoter/enhancers are known to the art. In an aspect, a disclosed promoter can be an endogenous promoter. In an aspect, a disclosed endogenous promoter can be an endogenous promoter/enhancer. In an aspect, a disclosed promoter or a disclosed promoter/enhancer can be used for constitutive and efficient expression of a disclosed isolated nucleic acid molecule. In an aspect, a disclosed promoter or a disclosed promoter/enhancer can be used for constitutive and efficient expression of a disclosed tumor antigen. In an aspect, a disclosed plasmid can be used to engineer cells to express a disclosed chimeric fusion protein.

In an aspect, a disclosed vector can be a viral vector or a non-viral vector. In an aspect, a disclosed non-viral vector can be a polymer-based vector, a peptide-based vector, a lipid nanoparticle, a solid lipid nanoparticle, or a cationic lipid-based vector.

In an aspect, a disclosed plasmid can comprise a transposon such as, for example, Sleeping Beauty and PiggyBac, both of which are known in the art. In an aspect, a first plasmid can be loaded with a disclosed nucleic acid sequence encoding a disclosed chimeric fusion protein, named transposon, surrounded by inverted repeats (IRs) that contain short direct repeats (DRs), while a second plasmid encodes the enzyme (transposase) that can recognize the sequences from the first plasmid and cut the transposon out of the first plasmid. Then the sequence encoding a disclosed chimeric fusion protein can be successfully delivered into the targeted cell (e.g., a T cell or a NK cell or a macrophage) cytoplasm and inserted randomly into TA dinucleotide base pairs of the recipient DNA sequence.

In an aspect, a disclosed plasmid can stimulate an effector cell mediated immune modulator response to PS-expressing tumor cells. In an aspect, a disclosed plasmid can induce a tumor reducing immune response. In an aspect, a disclosed plasmid can induce phagocytosis of cancer cells in the subject. In an aspect, a disclosed plasmid can cross-prime an anti-tumor T cell response. In an aspect, a disclosed plasmid can induce a tumor eliminating immune response. In an aspect, a disclosed plasmid can treat cancer. In an aspect, a disclosed plasmid can effect cell death of PS-expression cells. In an aspect, a disclosed plasmid can comprise improving the efficacy of one or more anti-cancer therapies and/or treatments.

In an aspect, a disclosed plasmid can be used to validate the efficacy and/or toxicity of disclosed chimeric fusion protein or pharmaceutical formulation thereof. In an aspect, validating the efficacy and/or toxicity of a disclosed chimeric fusion protein or pharmaceutical formulation thereof can comprise using an in vivo model and/or in vitro model.

### 6. Cells

Disclosed herein are cells transformed or transfected by one or more disclosed isolated nucleic acid molecules. Disclosed herein are cells transformed or transfected by an isolated nucleic acid molecule comprising a nucleic acid sequence encoding a disclosed chimeric fusion protein. Disclosed herein are cells transformed or transfected by an isolated nucleic acid molecule comprising a nucleic acid sequence encoding a disclosed chimeric fusion protein specific for phosphatidylserine (PS). Disclosed herein are cells transformed or transfected by an isolated nucleic acid molecule comprising a nucleic acid sequence encoding a chimeric fusion protein comprising a phosphatidylserine (PS) binding domain. Disclosed herein are cells transformed or transfected by an isolated nucleic acid molecule comprising a nucleic acid sequence encoding a chimeric fusion protein comprising a phosphatidylserine (PS) binding domain and an immunostimulatory domain. Disclosed herein are cells transformed or transfected by an isolated nucleic acid molecule comprising a nucleic acid sequence encoding a chimeric fusion protein comprising a phosphatidylserine (PS) binding domain operably linked an immunostimulatory domain. Disclosed herein are cells transformed or transfected by an isolated nucleic acid molecule comprising a nucleic acid sequence encoding a phosphatidylserine (PS) binding domain, a linker, and an immunostimulatory domain. Disclosed herein are cells transformed or transfected by an isolated nucleic acid molecule comprising a nucleic acid sequence encoding a chimeric fusion protein comprising a phosphatidylserine (PS) binding domain operably linked to an immunostimulatory domain, wherein the immunostimulatory domain further comprise a conjugated small molecule.

Disclosed herein are cells transformed or transfected by an isolated nucleic acid molecule comprising a nucleic acid sequence encoding a chimeric fusion protein comprising a secretion signal and a phosphatidylserine (PS) binding domain. Disclosed herein are cells transformed or transfected by an isolated nucleic acid molecule comprising a nucleic acid sequence encoding a chimeric fusion protein comprising a secretion signal, a phosphatidylserine (PS) binding domain, and an immunostimulatory domain. Disclosed herein are cells transformed or transfected by an isolated nucleic acid molecule comprising a nucleic acid sequence encoding a chimeric fusion protein comprising a secretion signal and a phosphatidylserine (PS) binding domain operably linked an immunostimulatory domain.

Disclosed herein are cells transformed or transfected by an isolated nucleic acid molecule comprising a nucleic acid sequence encoding a chimeric fusion protein comprising a secretion signal, a phosphatidylserine (PS) binding domain, a linker, and an immunostimulatory domain.

Disclosed herein are cells transformed or transfected by an isolated nucleic acid molecule comprising a nucleic acid sequence encoding a chimeric fusion protein comprising a secretion signal and a phosphatidylserine (PS) binding domain operably linked to an immunostimulatory domain, wherein the immunostimulatory domain further comprise a conjugated small molecule.

Disclosed herein are cells transformed or transfected by an isolated nucleic acid molecule comprising a nucleic acid sequence encoding a chimeric fusion protein comprising a PS-binding domain operably linked to an IgG1 Fc domain. Disclosed herein are cells transformed or transfected by an isolated nucleic acid molecule comprising a nucleic acid sequence encoding a chimeric fusion protein comprising a PS-binding domain operably linked to an IgG1 Fc domain and an additional immunostimulatory domain. Disclosed herein are cells transformed or transfected by an isolated nucleic acid molecule comprising a nucleic acid sequence encoding a chimeric fusion protein comprising a PS-binding domain operably linked to an IgG1 Fc domain and an additional immunostimulatory domain, wherein the additional immunostimulatory domain comprises the IgV domain of SIRPα or the extracellular domain of NKG2D protein.

Disclosed herein are cells transformed or transfected by an isolated nucleic acid molecule comprising a nucleic acid sequence encoding a chimeric fusion protein comprising a PS-binding domain operably linked to a single-chain antibody (scFV) to a human CD3 protein.

Disclosed herein are cells transformed or transfected by an isolated nucleic acid molecule comprising a nucleic acid sequence encoding a chimeric fusion protein comprising an Annexin-binding domain operably linked to a single-chain antibody (scFV) to a human CD3 protein.

Disclosed herein are cells transformed or transfected by an isolated nucleic acid molecule comprising a nucleic acid sequence encoding a single-chain antibody (scFV) to a human CD3 protein and one or more additional immunostimulatory domains.

Disclosed herein are cells transformed or transfected by an isolated nucleic acid molecule comprising a nucleic acid sequence encoding a chimeric fusion protein comprising an Annexin-binding domain operably linked to a single-chain antibody (scFV) to a human CD3 protein and one or more additional immunostimulatory domains.

Disclosed herein are cells transformed or transfected by an isolated nucleic acid molecule comprising a nucleic acid sequence encoding a chimeric fusion protein comprising the sequence set forth in SEQ ID NO: SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:99, SEQ ID NO:100, SEQ ID NO:101, SEQ ID NO:106, or a fragment thereof.

Disclosed herein are cells transduced by one or more disclosed viral vectors. Disclosed herein are cells transduced by a vector comprising a disclosed isolated nucleic acid molecule. Disclosed herein are cells transduced by a vector comprising a vector comprising an isolated nucleic acid molecule comprising a nucleic acid sequence encoding one or more disclosed chimeric fusion proteins. Disclosed herein are cells transduced by a vector comprising an isolated nucleic acid molecule comprising a nucleic acid sequence encoding a chimeric fusion protein comprising a phosphatidylserine (PS) binding domain. Disclosed herein are cells transduced by a vector comprising an isolated nucleic acid molecule comprising a nucleic acid sequence encoding a chimeric fusion protein comprising a phosphatidylserine (PS) binding domain and an immunostimulatory domain.

Disclosed herein are cells transduced by a vector comprising an isolated nucleic acid molecule comprising a nucleic acid sequence encoding a chimeric fusion protein comprising a phosphatidylserine (PS) binding domain operably linked an immunostimulatory domain. Disclosed herein are cells transduced by a vector comprising an isolated nucleic acid molecule comprising a nucleic acid sequence encoding a phosphatidylserine (PS) binding domain, a linker, and an immunostimulatory domain. Disclosed herein are cells transduced by a vector comprising an isolated nucleic acid molecule comprising a nucleic acid sequence encoding a chimeric fusion protein comprising a phosphatidylserine (PS) binding domain operably linked to an immunostimulatory domain, wherein the immunostimulatory domain further comprise a conjugated small molecule.

Disclosed herein are cells transduced by a vector comprising an isolated nucleic acid molecule comprising a nucleic acid sequence encoding a chimeric fusion protein comprising a secretion signal and a phosphatidylserine (PS) binding domain. Disclosed herein are cells transduced by a vector comprising an isolated nucleic acid molecule comprising a nucleic acid sequence encoding a chimeric fusion protein comprising a secretion signal, a phosphatidylserine (PS) binding domain, and an immunostimulatory domain.

Disclosed herein are cells transduced by a vector comprising an isolated nucleic acid molecule comprising a nucleic acid sequence encoding a chimeric fusion protein comprising a secretion signal and a phosphatidylserine (PS) binding domain operably linked an immunostimulatory domain. Disclosed herein are cells transduced by a vector comprising an isolated nucleic acid molecule comprising a nucleic acid sequence encoding a chimeric fusion protein comprising a secretion signal, a phosphatidylserine (PS) binding domain, a linker, and an immunostimulatory domain. Disclosed herein are cells transduced by a vector comprising an isolated nucleic acid molecule comprising a nucleic acid sequence encoding a chimeric fusion protein comprising a secretion signal and a phosphatidylserine (PS) binding domain operably linked to an immunostimulatory domain, wherein the immunostimulatory domain further comprise a conjugated small molecule.

Disclosed herein are cells transduced by a vector comprising an isolated nucleic acid molecule comprising a nucleic acid sequence encoding a chimeric fusion protein comprising a PS-binding domain operably linked to an IgG1 Fc domain. Disclosed herein are cells transduced by a vector comprising an isolated nucleic acid molecule comprising a nucleic acid sequence encoding a chimeric fusion protein comprising a PS-binding domain operably linked to an IgG1 Fc domain and an additional immunostimulatory domain. Disclosed herein are cells transduced by a vector comprising an isolated nucleic acid molecule comprising a nucleic acid sequence encoding a chimeric fusion protein comprising a PS-binding domain operably linked to an IgG1 Fc domain and an additional immunostimulatory domain, wherein the additional immunostimulatory domain comprises the IgV domain of SIRPα or the extracellular domain of NKG2D protein.

Disclosed herein are cells transduced by a vector comprising an isolated nucleic acid molecule comprising a nucleic acid sequence encoding a chimeric fusion protein comprising a PS-binding domain operably linked to a single-chain antibody (scFV) to a human CD3 protein. Disclosed herein are cells transduced by a vector comprising an isolated nucleic acid molecule comprising a nucleic acid sequence encoding a chimeric fusion protein comprising an Annexin-binding domain operably linked to a single-chain antibody (scFV) to a human CD3 protein. Disclosed herein are cells transduced by a vector comprising an isolated nucleic acid molecule comprising a nucleic acid sequence encoding a single-chain antibody (scFV) to a human CD3 protein and one or more additional immunostimulatory domains. Disclosed herein are cells transduced by a vector comprising an isolated nucleic acid molecule comprising a nucleic acid sequence encoding a chimeric fusion protein comprising an Annexin-binding domain operably linked to a single-chain antibody (scFV) to a human CD3 protein and one or more additional immunostimulatory domains.

Disclosed herein are cells transduced by a vector comprising an isolated nucleic acid molecule comprising a nucleic acid sequence encoding a chimeric fusion protein comprising the sequence set forth in SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:99, SEQ ID NO:100, SEQ ID NO:101, SEQ ID NO:106, or a fragment thereof.

Disclosed herein is a cell expressing a disclosed chimeric fusion protein, wherein the chimeric fusion protein an anti-PS binding domain, wherein the binding domain comprises Annexin A1 (ANXA1) or the PS-binding core domain, Annexin A2 (ANXA1), Annexin A3 (ANXA1), Annexin A4 (ANXA1), Annexin A5 (ANXA1), Annexin A6 (ANXA1), Annexin A7 (ANXA1), Annexin A8 (ANXA1), Annexin A8 Like 1 (ANXA1), Annexin A9 (ANXA1), Annexin A10 (ANXA1), Annexin A11 (ANXA1), Annexin A13 (ANXA1), Adhesion G Protein Coupled Receptor B1 (ADGRB1) or the extracellular domain thereof, Apolipoprotein H (APO-H), Coagulation Factor II (F2), Coagulation Factor VII (F7), Coagulation Factor IX (F9), Coagulation Factor X (F10), Growth Arrest Specific 6 (CAS6), Milk Fat Globule EGF And Factor V/VIII Domain Containing (MFGE8), Advanced Glycosylation End-Product Specific Receptor (AGER) or the extracellular domain thereof, Jumonji Domain-Containing Protein 6 (JMJD6); Protein S (PROS1), Hepatitis A Virus Cellular Receptor 1 (HAVCR1) or the extracellular domain thereof, Hepatitis A Virus Cellular Receptor 2 (HAVCR2) or the extracellular domain thereof, T Cell Immunoglobulin and Mucin Domain Containing (TIM-3) or the extracellular domain thereof, Protein Kinase C Alpha (PRKCA) or the C2 domain thereof, Synaptotagmin (SYT1) or the C2A domain thereof, Stabilin-1 (STAB1) or the extracellular domain thereof, Stabilin-2 (STAB2) or the extracellular domain thereof, or any combination thereof.

In an aspect, a disclosed cell can be transformed or transfected by an isolated nucleic acid molecule comprising a nucleic acid sequence encoding a tumor antigen. In an aspect, a disclosed cell can be transduced by a vector comprising an isolated nucleic acid molecule comprising a nucleic acid sequence encoding a disclosed chimeric fusion protein. In an aspect, following transformation, transfection, and/or transduction, a disclosed cell can express a disclosed chimeric fusion protein.

In an aspect, disclosed cells can comprise T cells or NK cells or macrophages. In an aspect, disclosed cells are immune cells. In an aspect, disclosed cells can comprise T cells, B cells, natural killer (NK) cells, dendritic cells, granulocytes, innate lymphoid cells, megakaryocytes, monocytes, macrophages, platelets, thymocytes, myeloid cells, or any combination thereof. In an aspect, disclosed cells can comprise any cell capable of generating a disclosed chimeric fusion protein. In an aspect, disclosed T cells and NK cells can be differentiated in vitro from a hematopoietic stem cell population (for example iPSCs) or can be obtained from a subject. In an aspect, T cells and NK cells can be obtained from, for example, peripheral blood mononuclear cells (PBMCs), bone marrow, lymph node tissue, cord blood, thymus tissue, tissue from a site of infection, ascites, pleural effusion, spleen tissue, tumors, or any combination thereof. In an aspect, disclosed T cells can be derived from one or more T cell lines available in the art. In an aspect, disclosed T cells can also be obtained from a unit of blood collected from a subject using any number of techniques known to the skilled person.

In an aspect, following transformation, transfection, and/or transduction, disclosed host cells can be used to generate a disclosed chimeric fusion protein.

### 7. Pharmaceutical Formulations

Disclosed herein is a pharmaceutical formulation comprising one or more chimeric fusion proteins, and one or more pharmaceutically acceptable carriers. Disclosed herein is a pharmaceutical formulation comprising one or more chimeric fusion proteins comprising a phosphatidylserine (PS) binding domain, and one or more pharmaceutically acceptable carriers. Disclosed herein is a pharmaceutical formulation comprising a chimeric fusion protein comprising a phosphatidylserine (PS) binding domain and an immunostimulatory domain, and one or more pharmaceutically acceptable carriers. Disclosed herein is a pharmaceutical formulation comprising a chimeric fusion protein comprising a phosphatidylserine (PS) binding domain operably linked an immunostimulatory domain, and one or more pharmaceutically acceptable carriers. Disclosed herein is a pharmaceutical formulation comprising a chimeric fusion protein comprising a phosphatidylserine (PS) binding domain, a linker, and an immunostimulatory domain, and one or more pharmaceutically acceptable carriers. Disclosed herein is a pharmaceutical formulation comprising a chimeric fusion protein comprising a phosphatidylserine (PS) binding domain operably linked to an immunostimulatory domain, wherein the immunostimulatory domain further comprise a conjugated small molecule, and one or more pharmaceutically acceptable carriers.

Disclosed herein is a pharmaceutical formulation comprising one or more chimeric fusion proteins comprising a secretion signal and a phosphatidylserine (PS) binding domain, and one or more pharmaceutically acceptable carriers. Disclosed herein is a pharmaceutical formulation comprising a chimeric fusion protein comprising a secretion signal, a phosphatidylserine (PS) binding domain, and an immunostimulatory domain, and one or more pharmaceutically acceptable carriers.

Disclosed herein is a pharmaceutical formulation comprising a chimeric fusion protein comprising a secretion signal and a phosphatidylserine (PS) binding domain operably linked an immunostimulatory domain, and one or more pharmaceutically acceptable carriers. Disclosed herein is a pharmaceutical formulation comprising a chimeric fusion protein comprising a secretion signal, a phosphatidylserine (PS) binding domain, a linker, and an immunostimulatory domain, and one or more pharmaceutically acceptable carriers. Disclosed herein is a pharmaceutical formulation comprising a chimeric fusion protein comprising a secretion signal and a phosphatidylserine (PS) binding domain operably linked to an immunostimulatory domain, wherein the immunostimulatory domain further comprise a conjugated small molecule, and one or more pharmaceutically acceptable carriers.

Disclosed herein is a pharmaceutical formulation comprising a chimeric fusion protein comprising a PS-binding domain operably linked to an IgG1 Fc domain, and one or more pharmaceutically acceptable carriers. Disclosed herein is a pharmaceutical formulation comprising a chimeric fusion protein comprising a PS-binding domain operably linked to an IgG1 Fc domain and an additional immunostimulatory domain, and one or more pharmaceutically acceptable carriers. Disclosed herein is a pharmaceutical formulation comprising a chimeric fusion protein comprising a PS-binding domain operably linked to an IgG1 Fc domain and an additional immunostimulatory domain, wherein the additional immunostimulatory domain comprises the IgV domain of SIRPα or the extracellular domain of NKG2D protein, and one or more pharmaceutically acceptable carriers. Disclosed herein is a pharmaceutical formulation comprising a chimeric fusion protein comprising a PS-binding domain operably linked to a single-chain antibody (scFV) to a human CD3 protein, and one or more pharmaceutically acceptable carriers. Disclosed herein is a pharmaceutical formulation comprising a chimeric fusion protein comprising an Annexin-binding domain operably linked to a single-chain antibody (scFV) to a human CD3 protein, and one or more pharmaceutically acceptable carriers. Disclosed herein is a pharmaceutical formulation comprising a chimeric fusion protein comprising a PS-binding domain operably linked to a single-chain antibody (scFV) to a human CD3 protein and one or more additional immunostimulatory domains, and one or more pharmaceutically acceptable carriers.

Disclosed herein is a pharmaceutical formulation comprising a chimeric fusion protein comprising an Annexin-binding domain operably linked to a single-chain antibody (scFV) to a human CD3 protein and one or more additional immunostimulatory domains, and one or more pharmaceutically acceptable carriers.

Disclosed herein is a pharmaceutical formulation comprising a chimeric fusion protein comprising the sequence set forth in SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO: 96, SEQ ID NO:97, SEQ ID NO:99, SEQ ID NO:100, SEQ ID NO:101, SEQ ID NO:106, or a fragment thereof, and one or more pharmaceutically acceptable carriers.

Disclosed herein is a pharmaceutical formulation comprising one or more isolated nucleic acid molecules comprising a nucleic acid sequence encoding one or more disclosed chimeric fusion proteins, and one or more pharmaceutically acceptable carriers. Disclosed herein is a pharmaceutical formulation comprising an isolated nucleic acid molecule comprising a nucleic acid sequence encoding a chimeric fusion protein comprising a phosphatidylserine (PS) binding domain, and one or more pharmaceutically acceptable carriers. Disclosed herein is a pharmaceutical formulation comprising an isolated nucleic acid molecule comprising a nucleic acid sequence encoding a chimeric fusion protein comprising a phosphatidylserine (PS) binding domain and an immunostimulatory domain, and one or more pharmaceutically acceptable carriers.

Disclosed herein is a pharmaceutical formulation comprising an isolated nucleic acid molecule comprising a nucleic acid sequence encoding a chimeric fusion protein comprising a phosphatidylserine (PS) binding domain operably linked an immunostimulatory domain, and one or more pharmaceutically acceptable carriers. Disclosed herein is a pharmaceutical formulation comprising an isolated nucleic acid molecule comprising a nucleic acid sequence encoding a phosphatidylserine (PS) binding domain, a linker, and an immunostimulatory domain, and one or more pharmaceutically acceptable carriers. Disclosed herein is a pharmaceutical formulation comprising an isolated nucleic acid molecule comprising a nucleic acid sequence encoding a chimeric fusion protein comprising a phosphatidylserine (PS) binding domain operably linked to an immunostimulatory domain, wherein the immunostimulatory domain further comprise a conjugated small molecule, and one or more pharmaceutically acceptable carriers.

Disclosed herein is a pharmaceutical formulation comprising an isolated nucleic acid molecule comprising a nucleic acid sequence encoding a chimeric fusion protein comprising a secretion signal and a phosphatidylserine (PS) binding domain, and one or more pharmaceutically acceptable carriers.

Disclosed herein is a pharmaceutical formulation comprising an isolated nucleic acid molecule comprising a nucleic acid sequence encoding a chimeric fusion protein comprising a secretion signal, a phosphatidylserine (PS) binding domain, and an immunostimulatory domain.

Disclosed herein is a pharmaceutical formulation comprising an isolated nucleic acid molecule comprising a nucleic acid sequence encoding a chimeric fusion protein comprising a secretion signal and a phosphatidylserine (PS) binding domain operably linked an immunostimulatory domain, and one or more pharmaceutically acceptable carriers.

Disclosed herein is a pharmaceutical formulation comprising an isolated nucleic acid molecule comprising a nucleic acid sequence encoding a chimeric fusion protein comprising a secretion signal, a phosphatidylserine (PS) binding domain, a linker, and an immunostimulatory domain, and one or more pharmaceutically acceptable carriers.

Disclosed herein is a pharmaceutical formulation comprising an isolated nucleic acid molecule comprising a nucleic acid sequence encoding a chimeric fusion protein comprising a secretion signal and a phosphatidylserine (PS) binding domain operably linked to an immunostimulatory domain, wherein the immunostimulatory domain further comprise a conjugated small molecule, and one or more pharmaceutically acceptable carriers.

Disclosed herein is a pharmaceutical formulation comprising an isolated nucleic acid molecule comprising a nucleic acid sequence encoding a chimeric fusion protein comprising a PS-binding domain operably linked to an IgG1 Fc domain, and one or more pharmaceutically acceptable carriers. Disclosed herein is a pharmaceutical formulation comprising an isolated nucleic acid molecule comprising a nucleic acid sequence encoding a chimeric fusion protein comprising a PS-binding domain operably linked to an IgG1 Fc domain and an additional immunostimulatory domain, and one or more pharmaceutically acceptable carriers. Disclosed herein is a pharmaceutical formulation comprising an isolated nucleic acid molecule comprising a nucleic acid sequence encoding a chimeric fusion protein comprising a PS-binding domain operably linked to an IgG1 Fc domain and an additional immunostimulatory domain, wherein the additional immunostimulatory domain comprises the IgV domain of SIRPα or the extracellular domain of NKG2D protein, and one or more pharmaceutically acceptable carriers.

Disclosed herein is a pharmaceutical formulation comprising an isolated nucleic acid molecule comprising a nucleic acid sequence encoding a chimeric fusion protein comprising a PS-binding domain operably linked to a single-chain antibody (scFV) to a human CD3 protein, and one or more pharmaceutically acceptable carriers. Disclosed herein is a pharmaceutical formulation comprising an isolated nucleic acid molecule comprising a nucleic acid sequence encoding a chimeric fusion protein comprising an Annexin-binding domain operably linked to a single-chain antibody (scFV) to a human CD3 protein, and one or more pharmaceutically acceptable carriers. Disclosed herein is a pharmaceutical formulation comprising an isolated nucleic acid molecule comprising a nucleic acid sequence encoding a single-chain antibody (scFV) to a human CD3 protein and one or more additional immunostimulatory domains, and one or more pharmaceutically acceptable carriers. Disclosed herein is an isolated nucleic acid molecule comprising a nucleic acid sequence encoding a chimeric fusion protein comprising an Annexin-binding domain operably linked to a single-chain antibody (scFV) to a human CD3 protein and one or more additional immunostimulatory domains, and one or more pharmaceutically acceptable carriers.

Disclosed herein is a pharmaceutical formulation comprising an isolated nucleic acid molecule comprising a nucleic acid sequence encoding a chimeric fusion protein comprising the sequence set forth in SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:99, SEQ ID NO:100, SEQ ID NO:101, SEQ ID NO:106, or a fragment thereof, and one or more pharmaceutically acceptable carriers.

Disclosed herein is a pharmaceutical formulation comprising a vector comprising a disclosed isolated nucleic acid molecule, and one or more pharmaceutically acceptable carriers. Disclosed herein is a pharmaceutical formulation comprising a vector comprising an isolated nucleic acid molecule comprising a nucleic acid sequence encoding one or more disclosed chimeric fusion proteins, and one or more pharmaceutically acceptable carriers. Disclosed herein is a pharmaceutical formulation comprising a vector comprising an isolated nucleic acid molecule comprising a nucleic acid sequence encoding a chimeric fusion protein comprising a phosphatidylserine (PS) binding domain, and one or more pharmaceutically acceptable carriers.

Disclosed herein is a pharmaceutical formulation comprising a vector comprising an isolated nucleic acid molecule comprising a nucleic acid sequence encoding a chimeric fusion protein comprising a phosphatidylserine (PS) binding domain and an immunostimulatory domain, and one or more pharmaceutically acceptable carriers.

Disclosed herein is a pharmaceutical formulation comprising a vector comprising an isolated nucleic acid molecule comprising a nucleic acid sequence encoding a chimeric fusion protein comprising a phosphatidylserine (PS) binding domain operably linked an immunostimulatory domain, and one or more pharmaceutically acceptable carriers.

Disclosed herein is a pharmaceutical formulation comprising a vector comprising an isolated nucleic acid molecule comprising a nucleic acid sequence encoding a phosphatidylserine (PS) binding domain, a linker, and an immunostimulatory domain, and one or more pharmaceutically acceptable carriers. Disclosed herein is a pharmaceutical formulation comprising a vector comprising an isolated nucleic acid molecule comprising a nucleic acid sequence encoding a chimeric fusion protein comprising a phosphatidylserine (PS) binding domain operably linked to an immunostimulatory domain, wherein the immunostimulatory domain further comprise a conjugated small molecule, and one or more pharmaceutically acceptable carriers.

Disclosed herein is a pharmaceutical formulation comprising a vector comprising an isolated nucleic acid molecule comprising a nucleic acid sequence encoding a chimeric fusion protein comprising a secretion signal and a phosphatidylserine (PS) binding domain, and one or more pharmaceutically acceptable carriers. Disclosed herein is a pharmaceutical formulation comprising a vector comprising an isolated nucleic acid molecule comprising a nucleic acid sequence encoding a chimeric fusion protein comprising a secretion signal, a phosphatidylserine (PS) binding domain, and an immunostimulatory domain, and one or more pharmaceutically acceptable carriers.

Disclosed herein is a pharmaceutical formulation comprising a vector comprising an isolated nucleic acid molecule comprising a nucleic acid sequence encoding a chimeric fusion protein comprising a secretion signal and a phosphatidylserine (PS) binding domain operably linked an immunostimulatory domain, and one or more pharmaceutically acceptable carriers. Disclosed herein is a pharmaceutical formulation comprising a vector comprising an isolated nucleic acid molecule comprising a nucleic acid sequence encoding a chimeric fusion protein comprising a secretion signal, a phosphatidylserine (PS) binding domain, a linker, and an immunostimulatory domain, and one or more pharmaceutically acceptable carriers.

Disclosed herein is a pharmaceutical formulation comprising a vector comprising an isolated nucleic acid molecule comprising a nucleic acid sequence encoding a chimeric fusion protein comprising a secretion signal and a phosphatidylserine (PS) binding domain operably linked to an immunostimulatory domain, wherein the immunostimulatory domain further comprise a conjugated small molecule, and one or more pharmaceutically acceptable carriers.

Disclosed herein is a pharmaceutical formulation comprising a vector comprising an isolated nucleic acid molecule comprising a nucleic acid sequence encoding a chimeric fusion protein comprising a PS-binding domain operably linked to an IgG1 Fc domain, and one or more pharmaceutically acceptable carriers. Disclosed herein is a pharmaceutical formulation comprising a vector comprising an isolated nucleic acid molecule comprising a nucleic acid sequence encoding a chimeric fusion protein comprising a PS-binding domain operably linked to an IgG1 Fc domain and an additional immunostimulatory domain, and one or more pharmaceutically acceptable carriers. Disclosed herein is a pharmaceutical formulation comprising a vector comprising an isolated nucleic acid molecule comprising a nucleic acid sequence encoding a chimeric fusion protein comprising a PS-binding domain operably linked to an IgG1 Fc domain and an additional immunostimulatory domain, wherein the additional immunostimulatory domain comprises the IgV domain of SIRPα or the extracellular domain of NKG2D protein, and one or more pharmaceutically acceptable carriers.

Disclosed herein is a pharmaceutical formulation comprising a vector comprising an isolated nucleic acid molecule comprising a nucleic acid sequence encoding a chimeric fusion protein comprising a PS-binding domain operably linked to a single-chain antibody (scFV) to a human CD3 protein, and one or more pharmaceutically acceptable carriers. Disclosed herein is a pharmaceutical formulation comprising a vector comprising an isolated nucleic acid molecule comprising a nucleic acid sequence encoding a chimeric fusion protein comprising an Annexin-binding domain operably linked to a single-chain antibody (scFV) to a human CD3 protein, and one or more pharmaceutically acceptable carriers. Disclosed herein is a pharmaceutical formulation comprising a vector comprising an isolated nucleic acid molecule comprising a nucleic acid sequence encoding a single-chain antibody (scFV) to a human CD3 protein and one or more additional immunostimulatory domains, and one or more pharmaceutically acceptable carriers. Disclosed herein is a pharmaceutical formulation comprising a vector comprising an isolated nucleic acid molecule comprising a nucleic acid sequence encoding a chimeric fusion protein comprising an Annexin-binding domain operably linked to a single-chain antibody (scFV) to a human CD3 protein and one or more additional immunostimulatory domains, and one or more pharmaceutically acceptable carriers.

Disclosed herein is a pharmaceutical formulation comprising a vector comprising an isolated nucleic acid molecule comprising a nucleic acid sequence encoding a chimeric fusion protein comprising the sequence set forth in SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:99, SEQ ID NO:100, SEQ ID NO:101, SEQ ID NO:106, or a fragment thereof, and one or more pharmaceutically acceptable carriers.

Disclosed herein is a pharmaceutical formulation comprising a vector comprising an isolated nucleic acid molecule comprising a nucleic acid sequence encoding a disclosed bispecific chimeric fusion protein as set forth in FIG. 5A-FIG. 5H, and one or more pharmaceutically acceptable carriers. Disclosed herein is a pharmaceutical formulation comprising a vector comprising an isolated nucleic acid molecule comprising a nucleic acid sequence encoding a disclosed bispecific chimeric fusion protein as set forth in FIG. 5A-FIG. 5H, wherein the PS-binding domain comprises any disclosed PS-binding domain and wherein the anti-CD3 binding domain comprises any disclosed binding domain, and one or more pharmaceutically acceptable carriers.

In an aspect, a disclosed pharmaceutical formulation can comprise (i) one or more active agents, (ii) biologically active agents, (iii) one or more pharmaceutically active agents, (iv) one or more immune-based therapeutic agents, (v) one or more clinically approved agents, or (vi) a combination thereof.

In an aspect, a disclosed pharmaceutical formulation can further comprise one or more anti-inflammatory agents. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

In an aspect, NSAIDs can comprise ibuprofen, naproxen, naproxen sodium, Cox-2 inhibitors such as rofecoxib and celecoxib, sialylates, or any combination thereof. In an aspect, analgesics can comprise acetaminophen, oxycodone, tramadol, proporxyphene hydrochloride, or any combination thereof. In an aspect, glucocorticoids can comprise cortisone, dexamethasone, hydrocortisone, methylprednisolone, prednisolone, prednisone, or any combination thereof. Exemplary biological response modifiers include molecules directed against cell surface markers (e.g., CD4, CD5, etc.), cytokine inhibitors, such as the TNF antagonists (e.g., etanercept, adalimumab, and infliximab, chemokine inhibitors and adhesion molecule inhibitors. In an aspect, biological response modifiers can comprise monoclonal antibodies as well as recombinant forms of molecules. In an aspect, exemplary disease-modifying anti-rheumatic drugs (DMARDs) can comprise include azathioprine, cyclophosphamide, cyclosporine, methotrexate, penicillamine, leflunomide, sulfasalazine, hydroxychloroquine, Gold (oral (auranofin) and intramuscular), minocycline, or any combination thereof.

In an aspect, a disclosed chemotherapeutic agent in a disclosed pharmaceutical formulation can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof. In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, irinotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

In an aspect, a disclosed pharmaceutical formulation can comprise an anti-chemokine therapy that enhances the resident memory T cell formations in tumor-free tissues. In an aspect, a disclosed anti-chemokine therapy can comprise one or more antibodies against CCL1, CCL2, CCL4, CCL17, CCL19, CCL21, CCL22, CCL25, CXCL9, CXCL10, CXCL11, CXCL12, CXCL13, CCR2, CCR5, CCR7, CCR8, CCR9, CXCR3, CXCR4, CXCR5, CX3CL1, CX3CR1, or any combination thereof.

In an aspect, a disclosed pharmaceutical formulation can further comprise abagovomab, adecatumumab, afutuzumab, alemtuzumab, altumomab, amatuximab, anatumomab, arcitumomabm bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farietuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, namatumab, naptumomab, necitumumab, nimotuzumab, nofetumomab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, 3F8, or any combination thereof.

In an aspect, a disclosed pharmaceutical formulation can stimulate an effector cell mediated immune response to PS-expressing tumor cells. In an aspect, a disclosed pharmaceutical formulation can restore one or more aspects of cellular homeostasis and/or cellular functionality and/or metabolic dysregulation in a subject having cancer. In an aspect, metabolic dysregulation can be associated with cancer or cancerous cells. In an aspect, following administration of a disclosed pharmaceutical formulation, cell death of PS-expressing cancer cells can be effected.

In an aspect, a disclosed pharmaceutical formulation can be prepared for systemic or direct administration. In an aspect, a disclosed pharmaceutical formulation can be prepared for oral administration, intravenous administration, intratumoral administration, intraperitoneal administration, or any combination thereof. In an aspect, a disclosed pharmaceutical formulation can be prepared for any method of administration disclosed herein. In an aspect, a disclosed pharmaceutical formulation can be prepared for administration via multiple routes either concurrently or sequentially. For example, in an aspect, a disclosed pharmaceutical formulation can be first administered intratumorally and then be administered intravenously. In an aspect, a disclosed pharmaceutical formulation can be first administered intratumorally and then be administered orally. A skilled clinical can determine the best route of administration for a subject at a given time.

In an aspect, a disclosed pharmaceutical formulation can comprise one or more immune modulators. In an aspect, a disclosed pharmaceutical formulation can comprise one or more proteasome inhibitors. In an aspect, a disclosed pharmaceutical formulation can comprise one or more immunosuppressives or immunosuppressive agents. In an aspect, an immunosuppressive agent can be anti-thymocyte globulin (ATG), cyclosporine (CSP), mycophenolate mofetil (MMF), or a combination thereof. In an aspect, a disclosed pharmaceutical formulation can comprise an anaplerotic agent (such as, for example, C7 compounds like triheptanoin or MCT).

In an aspect, a disclosed pharmaceutical formulation can comprise an RNA therapeutic. An RNA therapeutic can comprise RNA-mediated interference (RNAi) and/or antisense oligonucleotides (ASO). In an aspect, a disclosed RNA therapeutic can be directed at any protein or enzyme that is overexpressed or is overactive due to a missing, deficient, and/or mutant protein or enzyme (such as, for example, a missing, deficient, and/or mutant protein or enzyme related to cancer and/or associated with cancerous cells). In an aspect, a disclosed RNA therapeutic can be directed at any protein or enzyme that is overexpressed or is overactive and related to cancer and/or associated with cancerous cells.

In an aspect, a disclosed therapeutically effective amount or effective dose of a disclosed chimeric fusion protein or pharmaceutical formulation thereof can be about 0.01 μg/kg to about 10 mg/kg per subject.

In an aspect, a disclosed pharmaceutically acceptable carrier can comprise any disclosed carrier. In an aspect, a disclosed pharmaceutically acceptable carrier can comprise any disclosed excipient.

In an aspect, a disclosed pharmaceutical formulation can stimulate an effector cell mediated immune modulator response to PS-expressing tumor cells. In an aspect, a disclosed pharmaceutical formulation can induce a tumor reducing immune response. In an aspect, a disclosed pharmaceutical formulation can induce phagocytosis of cancer cells in the subject. In an aspect, a disclosed pharmaceutical formulation can cross-prime an anti-tumor T cell response. In an aspect a disclosed pharmaceutical formulation can induce a tumor eliminating immune response. In an aspect, a disclosed pharmaceutical formulation can treat cancer.

In an aspect, a disclosed pharmaceutical formulation can induce cell death in the targeted cell. In an aspect, a disclosed pharmaceutical formulation can stimulate an effector cell mediated immune modulator response to PS-expressing tumor cells. In an aspect, a disclosed pharmaceutical formulation can induce a tumor reducing immune response. In an aspect, a disclosed pharmaceutical formulation can induce phagocytosis of cancer cells in the subject. In an aspect, a disclosed pharmaceutical formulation can cross-prime an anti-tumor T cell response. In an aspect, a disclosed pharmaceutical formulation can induce a tumor eliminating immune response. In an aspect, a disclosed pharmaceutical formulation can treat cancer. In an aspect, a disclosed pharmaceutical formulation can improve and/or extend the survivability of the subject, can improve a subject's quality of life, can increase and/or prolong a subject's life span, or any combination thereof.

### 8. Animals

Disclosed herein are animals used to validate the efficacy and/or safety of one or more disclosed chimeric fusion proteins, one or more disclosed antibody drug conjugates, one or more disclosed isolated nucleic acid molecules, one or more disclosed vectors, one or more disclosed pharmaceutical formulations, or any combination thereof. In an aspect, a disclosed animal can be treated with one or more disclosed chimeric fusion proteins, one or more disclosed antibody drug conjugates, one or more disclosed isolated nucleic acid molecules, one or more disclosed vectors, one or more disclosed pharmaceutical formulations, or any combination thereof. In an aspect, animals can be assessed and/or monitored for one or more biological and/or chemical functions prior to treatment, during treatment, after treatment, or any combination thereof, wherein treatment comprises administering one or more disclosed chimeric fusion proteins, one or more disclosed antibody drug conjugates, one or more disclosed isolated nucleic acid molecules, one or more disclosed vectors, one or more disclosed pharmaceutical formulations, or any combination thereof. In an aspect, a disclosed treated subject can be a mouse or a rat. In an aspect, a disclosed treated subject can be a transgenic mouse or a transgenic rat. In an aspect, a disclosed treated subject can have one or more types of cancers and/or tumors (such as, for example, hematological cancer).

### E. Methods of Treating a Hematological Cancer

Disclosed herein is a method of treating a hematological cancer, the method comprising treating a subject in need thereof by administering to the subject in need thereof a therapeutically effective amount of a disclosed chimeric fusion protein or a pharmaceutical formulation thereof. Disclosed herein is a method of treating a hematological cancer, the method comprising treating a subject in need thereof by administering to the subject in need thereof a therapeutically effective amount of a disclosed chimeric fusion protein or a pharmaceutical formulation thereof. Disclosed herein is a method of treating a hematological cancer, the method comprising treating a subject in need thereof by administering to the subject in need thereof a therapeutically effective amount of a disclosed chimeric fusion protein targeting phosphatidylserine (PS) or a pharmaceutical formulation thereof.

Disclosed herein is a method of treating a hematological cancer, the method comprising treating a subject in need thereof by administering to the subject in need thereof a therapeutically effective amount of a disclosed chimeric fusion protein targeting phosphatidylserine-expressing tumor cells or a pharmaceutical formulation thereof. In an aspect, wherein the disclosed chimeric fusion protein targets phosphatidylserine (PS)-expressing cancer cells. In an aspect, wherein the disclosed chimeric fusion protein targets phosphatidylserine (PS)-expressing hematological cancer cells. In an aspect, disclosed PS-expressing cancer cells can be lymphoma cells, leukemia cells, and multiple myeloma cells. In an aspect, disclosed PS-expressing cancer cells can be acute myeloid leukemia (AML) or myelodysplastic syndrome. In an aspect, disclosed PS-expressing cancer cells can be blood borne.

In an aspect, a subject can be treatment-naïve. In an aspect, a subject has received one or more treatments prior to the administering of a disclosed chimeric fusion protein or pharmaceutical formulation thereof. In an aspect, one or more treatments can comprise one or more anti-cancer therapies and/or treatments.

In an aspect, a subject can have, be diagnosed with, or be suspected of having one or more cancers. In an aspect, a disclosed method of treating a hematological cancer can further comprise diagnosing the subject as have cancer or cancerous cells. In an aspect, a disclosed method of treating a hematological cancer can further comprise diagnosing the subject as having a hematological cancer or cancerous hematological cells. In an aspect, a disclosed hematological cancer can comprise lymphoma, myeloma, leukemia, or any combination thereof.

In an aspect, a subject can have, be diagnosed with, or be suspected of having one or more cancers. In an aspect, a subject can have, be diagnosed with, or be suspected of having a hematological cancer and one or more other cancers. In an aspect, other cancers can comprise ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

In an aspect, a disclosed method of treating a hematological cancer can further comprise collecting one or more blood and/or biological samples from a subject at the same time or at different times. For example, in an aspect, a blood sample and/or a biological sample can be collected from a subject at a pre-determined interval. In an aspect, a pre-determined interval can be once a week, once every 2 weeks, once every 3 weeks, once every 4 weeks, once every 5 weeks, once every 6 weeks, once every 7 weeks, once every 8 weeks, or at a longer interval. In an aspect, a pre-determined interval can be once a month, once every 2 months, once every 3 months, once every 5 months, once every 5 months, once every 6 months, or at a longer interval. In an aspect, a blood sample and/or a biological sample can be collected from a subject prior to treatment, during treatment, after treatment, or any combination thereof. In an aspect, a blood and/or a biological sample can be collected from a subject at any time deemed medically and/or clinically appropriate by the skilled clinician.

In an aspect, a therapeutically effective amount or effective dose or effective amount or therapeutically effective dosage of a disclosed chimeric fusion protein or pharmaceutical formulation thereof can be any amount that, when used alone or in combination with another therapeutic agent, can attack and destroy PS-expressing tumor cells. In an aspect, a therapeutically effective amount or effective dose or effective amount or therapeutically effective dosage of a disclosed chimeric fusion protein or pharmaceutical formulation thereof can be any amount that, when used alone or in combination with another therapeutic agent, can attack and destroy PS-expressing tumor cells. In an aspect, a therapeutically effective amount or effective dose or effective amount or therapeutically effective dosage of a disclosed chimeric fusion protein or pharmaceutical formulation thereof can be any amount that can enhance the success rate of one or more conventional cytotoxic cancer therapies against PS-expressing tumor cells.

In an aspect, a disclosed therapeutically effective amount or effective dose of a disclosed chimeric fusion protein or pharmaceutical formulation thereof can be about 0.01 μg/kg to about 10 mg/kg per subject.

In an aspect, a therapeutically effective amount or effective dose or effective amount or therapeutically effective dosage of a disclosed chimeric fusion protein or pharmaceutical formulation thereof can protect a subject against the onset of a disease and/or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

In an aspect, administering a disclosed chimeric fusion protein or pharmaceutical formulation thereof can comprise systemic or direct administration. In an aspect, administering can comprise oral administration, intravenous administration, intratumoral administration, intraperitoneal administration, intracranial administration, subcutaneous administration, intradermal administration, intrathecal administration, or any combination thereof. In an aspect, administering a disclosed chimeric fusion protein or pharmaceutical formulation thereof can be administered by any method of administration disclosed herein. In an aspect, a disclosed chimeric fusion protein or pharmaceutical formulation thereof can be administered via multiple routes either concurrently or sequentially.

For example, in an aspect, a disclosed chimeric fusion protein or pharmaceutical formulation thereof can be first administered intratumorally and then be administered intravenously. In an aspect, a disclosed chimeric fusion protein or pharmaceutical formulation thereof can be first administered intratumorally and then be administered orally. A skilled clinician can determine the best route of administration for a subject at a given time.

In an aspect, a disclosed method can comprise repeating the administering of a disclosed chimeric fusion protein or pharmaceutical formulation thereof.

In an aspect, a disclosed method of treating a hematological cancer can comprise protecting the subject from metastasis. In an aspect, a disclosed method of treating a hematological cancer can comprise reducing the risk of developing metastasis. In an aspect, a disclosed method of treating a hematological cancer can comprise preventing or inhibiting metastasis.

In an aspect, a disclosed method can comprise monitoring the subject for adverse effects. In an aspect, in the absence of adverse effects, a disclosed method can comprise continuing to treat the subject. In an aspect, continuing to treat the subject can comprise continuing to administer to the subject a disclosed chimeric fusion protein or pharmaceutical formulation thereof. In an aspect, in the presence of adverse effects, a disclosed method can comprise modifying one or more steps of the method. In an aspect, modifying one or more steps of a disclosed method can comprise modifying the administering step. In an aspect, modifying the administering step can comprise changing the amount of a disclosed chimeric fusion protein or pharmaceutical formulation thereof administered to the subject, changing the frequency of administration of a disclosed chimeric fusion protein or pharmaceutical formulation thereof, changing the duration of administration of a disclosed chimeric fusion protein or pharmaceutical formulation thereof, changing the route of administration of a disclosed chimeric fusion protein or pharmaceutical formulation thereof, or any combination thereof.

In an aspect, a disclosed method of treating a hematological cancer can further comprise administering to the subject an immune checkpoint inhibitor (e.g., an anti-PD1 molecule). In an aspect, a disclosed anti-PD1 molecule can comprise an anti-PD1 antibody, an anti-PDL1 antibody, or any combination thereof. In an aspect, a disclosed anti-PD1 antibody can comprise a monoclonal antibody, a humanized monoclonal antibody, or a fragment thereof. In an aspect, a disclosed anti-PD1 antibody can comprise a polyclonal antibody, a humanized polyclonal antibody, or a fragment thereof. In an aspect, a disclosed anti-PD1 antibody can comprise any antibody or antibody fragment that specifically recognizes PD1. In an aspect, a disclosed anti-PDL1 antibody can comprise a monoclonal antibody, a humanized monoclonal antibody, or a fragment thereof. In an aspect, a disclosed anti-PDL1 antibody can comprise a polyclonal antibody, a humanized polyclonal antibody, or a fragment thereof. In an aspect, a disclosed anti-PDL1 antibody can comprise any antibody or antibody fragment that specifically recognizes PDL1. Antibodies and methods of preparing antibodies are known in the art. Similarly, recombinant antibodies and methods of preparing recombinant antibodies are known in the art.

In an aspect, a disclosed method of treating a hematological cancer can further comprise repeating the administering of the anti-PD1 molecule. In an aspect, a disclosed anti-PD1 molecule can be administered prior to, concurrent with, or after the administration of the interfering molecule. In an aspect of a disclosed method of treating a hematological cancer administering a disclosed anti-PD1 molecule can comprise systemic or direct administration. In an aspect, administering a disclosed anti-PD1 molecule can comprise intravenous administration, intratumoral administration, intraperitoneal administration, or any combination thereof. In an aspect, administering a disclosed can be administered by any method of administration disclosed herein. In an aspect, a disclosed anti-PD1 molecule can be administered via multiple routes either concurrently or sequentially. For example, in an aspect, a disclosed anti-PD1 molecule can be first administered intratumorally and then be administered intravenously. In an aspect, administering a disclosed anti-PD1 molecule can be first administered intratumorally and then be administered orally. A skilled clinician can determine the best route of administration for a subject at a given time.

For example, in an aspect, a disclosed anti-PD1 molecule can be administered about 3 months, about 2 months, or about 1 month prior to the administering of a disclosed chimeric fusion protein or pharmaceutical formulation thereof. In an aspect, a disclosed anti-PD1 molecule can be administered about 8 weeks, about 7 weeks, about 6 weeks, about 5 weeks, about 4 weeks, about 3 weeks, about 2 weeks, or about 1 week prior to the administering of a disclosed chimeric fusion protein or pharmaceutical formulation thereof. In an aspect, a disclosed anti-PD1 molecule can be administered about 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day prior to the administering of a disclosed chimeric fusion protein or pharmaceutical formulation thereof. In an aspect, a disclosed anti-PD1 molecule can be administered about 24, 23, 22, 21, 20, 19, 18, 17, 16, 15, 14, 13, 12, 11, 10, 9, 8, 7, 6, 5, 4, 3, 2, or 1 hours prior to the administering of a disclosed chimeric fusion protein or pharmaceutical formulation thereof.

In an aspect, a disclosed method of treating a hematological cancer can further comprise repeating the administering of a disclosed anti-PD1 molecule. In an aspect, a disclosed anti-PD1 molecule can be administered prior to, concurrent with, or after the administration of the disclosed chimeric fusion protein.

In an aspect, a disclosed method of treating a hematological cancer can comprise administering to the subject one or more additional anti-cancer therapies. Anti-cancer therapies are known to the art. In an aspect, a disclosed anti-cancer therapy can comprise endocrine therapy, radiotherapy, hormone therapy, gene therapy, thermal therapy, ultrasound therapy, or any combination thereof. In an aspect, a disclosed anti-cancer therapy can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof. In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), axitinib (Inlyta), bevacizumab (Avastin), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, irinotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

In an aspect, a disclosed method of treating a hematological cancer can further comprise repeating the administering of a disclosed chemotherapeutic agent. In an aspect, a disclosed chemotherapeutic agent can be administered prior to, concurrent with, or after the administration of the disclosed chimeric fusion protein. The table below provides a non-comprehensive listing of treatments of hematologic cancers as known to the art.

| Classification | Mechanism of Action | Examples |
|---|---|---|
| Antitumor antibiotics | Interact directly with DNA in the nucleus of cells, | Bleomycin sulfate (Blenoxane ®) |
| | interfering with cell survival | Daunorubicin (Cerubidine ®) |
| | | Idarubicin (Idamycin ®) |
| | | Doxorubicin (Adriamycin ®) |
| | | Mitoxantrone (Novantrone ®) |
| Antimetabolites | Block cells' ability to form RNA or DNA, preventing | Cladribine (Leustatin ®, 2-CdA) |
| | cell growth and accelerating cell death | Cytarabine (cytosine arabinoside, ARA-C, Cyto-sar-U ®) |
| | | Fludarabine (Fludara ®) |
| | | Hydroxyurea (Hydrea ®, Droxia ®) |
| | | 6-Mercaptopurine (Purinethol ®) |
| | | Methotrexate |
| | | 6-Thioguanine (Thioguanine ®, Tabloid ®) |
| | | Azacitidine (Vidaza ®) |
| | | Decitabine (Dacogen ®) |
| | | Clofarabine (Clolar ®) |
| Immunomodulators | Exact mechanism of action is unclear; immune, | Interferon (Roferon ® A, Intron ® A) |
| | cytotoxic, and antiangiogenic effects | Pegylated interferon (PEG IFN) |
| | | Thalidomide (Thalomid ®) |
| | | Lenalidomide (Revlimid ®) |
| Histone deacetylase | Modulate chromatin structure and gene expression; | Vorinostat (Zolinza ®) |
| inhibitors | induce cell growth arrest, cell differentiation, and death | |
| | of leukemia cells | |
| Corticosteroids | Cytotoxic activity against lymphoma and leukemia cells | Dexamethasone (Decadron ®) |
| | | Methylprednisolone (Medrol ®) |
| | | Prednisone (Deltasone ®) |
| Bisphosphonates | Block the reabsorption of bone in myeloma and have | Pamidronate (Aredia ®) |
| | direct effects on myeloma cells | Zoledronic acid (Zometa ®) |
| Plant alkaloids | Act on certain proteins (enzymes) in the cell nucleus that | Etoposide (VP-16, VePesid ®, Etopophos ®, Toposar ®) |
| | normally repair injury to DNA (DNA-repair enzyme | Teniposide (VM-26, Vumon ®) |
| | inhibitors) | Topotecan (Hycamtin ®) |
| | Impair structures in the cell that are required for cells to | Vinblastine (Velban ®) |
| | divide into two daughter cells (block mitosis) | Vincristine (Oncovin ®) |
| | | Paclitaxel (Taxol ®) |
| Alkylating agents | Act on the breakdown of proteins in the proteasome, a | Bendamustine (Treanda ®) |
| | key cell function; used for multiple myeloma | Busulfan (Myleran ®, Busulfex ®) |
| | | Carboplatin (Paraplatin ®) |
| | | Carmustine (BCNU, BiCNU ®) |
| | | Chlorambucil (Leukeran ®) |
| | | Cisplatin (Platinol ®) |
| | | Cyclophosphamide (Cytoxan ®, Neosar ®) |
| | | Dacarbazine (DTIC-Dome ®) |
| | | Ifosfamide (Ifex ®) |
| | | Lomustine (CCNU ®, CeeNU ®) |
| | | Mechlorethamine (nitrogen mustard, Mustargen ®) |
| | | Melphalan (Alkeran ®) |
| | | Procarbazine (Matulane ®) |
| | | Bortezomib (Velcade ®) |
| Proteasome inhibitors | Act on the breakdown of proteins in the proteasome, a | Bortezomib (Velcade ®) |
| | key cell function; used for multiple myeloma | |
| Monoclonal antibodies | Target specific antigens on cancer cells | Rituximab (Rituxan ®) |
| | | Yttrium-90-ibritumomab tiuxetan (Zevalin ®) |
| | | Tositumomab (Bexxar ®) |
| | | Ofatumumab (Arzerra ®) |
| Tyrosine kinase | Block specific mutant proteins that initiate malignant | Imatinib mesylate (Gleevec ®) |
| inhibitors | cell transformation | Dasatinib (Sprycel ®) |
| | | Nilotinib (Tasigna ®) |
| Cell-maturing agents | Induce maturation of leukemia cells | Tretinoin (all-trans-retinoic acid [ATRA], Vesanoid ®) |
| | | Arsenic trioxide (Trisenox ®) |
| Janus kinase inhibitor | Janus kinase inhibitor (JAK1 and JAK2) | Ruxolitinib (Jakafi ®) |
| Phototherapy | Activated by ultraviolet light to kill skin lymphoma cells | Psoralen |

In an aspect, a disclosed method of treating a hematological cancer can comprise administering to the subject an anti-chemokine therapy. In an aspect, a disclosed anti-chemokine therapy can comprise one or more antibodies against CCL1, CCL2, CCL4, CCL17, CCL19, CCL21, CCL22, CCL25, CXCL9, CXCL10, CXCL11, CXCL12, CXCL13, CCR2, CCR5, CCR7, CCR8, CCR9, CXCR3, CXCR4, CXCR5, CX3CL1, CX3CR1, or any combination thereof. In an aspect, a disclosed method of treating a hematological cancer can further comprise repeating the administering of a disclosed anti-PD1 molecule. In an aspect, a disclosed anti-chemokine molecule can be administered prior to, concurrent with, or after the administration of the disclosed chimeric fusion protein.

In an aspect, a disclosed method of treating a hematological cancer can comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. CRISPR/Cas9 systems and methods are known to the art. In an aspect, a disclosed method of treating a hematological cancer can further comprise repeating the administering of a disclosed anti-oligonucleotide therapeutic agent. In an aspect, a disclosed anti-oligonucleotide therapeutic agent can be administered prior to, concurrent with, or after the administration of the disclosed chimeric fusion protein.

In an aspect, a disclosed method of treating a hematological cancer can further comprise preventing or inhibiting metastasis of cancer cells. In an aspect, a disclosed method can comprise a 10%, 20%, 30%, 40%, 50%, 60%, 70%, 80%, 90%, 100%, or any amount of decrease and/or reduction in the risk of and/or actual metastasis of cancer cells when compared to a control subject (such as, for example, a subject that has not received a disclosed treatment (e.g., a disclosed chimeric fusion protein or a pharmaceutical formulation thereof)). In an aspect, preventing or inhibiting metastasis of cancer cells can comprise a 10-20%, 20-30%, 30-40%, 40-50%, 50-60%, 60-70%, 70-80%, 80-90%, or 90-100% or any amount of decrease and/or reduction in the risk of and/or actual metastasis of cancer cells when compared to a control subject (such as, for example, a subject that has not received a disclosed treatment (e.g., a disclosed chimeric fusion protein or a pharmaceutical formulation thereof).

In an aspect, a disclosed method of treating a hematological cancer can comprise surgically resecting the tumor and/or cancer cells from the subject. In an aspect, following resecting the tumor and/or cancer cells from the subject, a disclosed method of treating a hematological cancer can comprise continuing to administer to the subject a therapeutically effective amount of a disclosed chimeric fusion protein or a pharmaceutical formulation thereof and continuing to administer to the subject a therapeutically effective amount of an anti-PD1 molecule, a disclosed anti-chemokine therapy, a disclosed chemotherapeutic agent, any disclosed therapeutic agent, or any combination thereof.

In an aspect, a disclosed method of treating a hematological cancer can further comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed method of treating a hematological cancer can restore one or more aspects of cellular homeostasis and/or cellular functionality and/or metabolic dysregulation in a subject, such as, for example, a subject having a hematological cancer or cancerous cells. In an aspect, a disclosed chimeric fusion protein or pharmaceutical formulation thereof can restore one or more aspects of cellular homeostasis and/or cellular functionality and/or metabolic dysregulation in a subject having a hematological cancer. In an aspect, metabolic dysregulation can be associated with a hematological cancer or cancerous cells. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cancer; (vii) reversing, inhibiting, preventing, stabilizing, and/or slowing the rate of progression of a cancer, or (viii) any combination thereof. In an aspect, restoring one or more aspects of cellular homeostasis can comprise improving, enhancing, restoring, and/or preserving one or more aspects of cellular structural and/or functional integrity. In an aspect, restoration can be a partial or incomplete restoration. In an aspect, restoration can be complete or near complete restoration such that the level of expression, activity, and/or functionality is similar to that of a wild-type or control level. In an aspect, restoring one or more aspects of cellular homeostasis and/or cellular functionality can comprise preventing or inhibiting metastasis of cancer cells in the subject.

In an aspect of a disclosed method of treating a hematological cancer, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are provided below.

| Regulated Variable | Sensor |
|---|---|
| Blood Pressure/Blood | Aortic Body (Aorta), Carotid Body (Carotid |
| Volume/Na⁺ Conc./ | Artery), Atrial Volume Receptors (Heart), |
| | Juxtaglomerular Apparatus (Kidney) |
| Ca²⁺/Mg²⁺/PO₄³⁻ Conc. | Chief Cells (Parathyroid Gland) |
| Glucose | Islet of Langerhans (Pancreas) |
| Osmolarity | Circumventricular Organs (Hypothalamus) |
| pO₂, pCO₂, and pH | Aortic Body (Aorta), Carotid Body (Carotid |
| | Artery), Ventrolateral Medulla (Medulla) |
| Temperature | Thermosensory neurons (Skin), Preoptic Area |
| | (Hypothalamus) |

In an aspect of a disclosed method of treating a hematological cancer, techniques to monitor, measure, and/or assess the status of a subject's hematological disease and/or disorder and/or cancer can comprise qualitative (or subjective) means as well as quantitative (or objective) means. In an aspect, microscopy can be used for the visualization of cells to determine morphology and staining characteristics. In an aspect, immunohistochemistry can be used to identify specific molecules in different kinds of cells and/or tissues. The tissue is treated with antibodies that bind to the specific molecule. These are made visible under a microscope by using a color reaction, a radioisotope, or a fluorescent dye. In an aspect, flow cytometry can be used to sort and collect cells for additional analyses as flow cytometer is capable of rapid, quantitative, multiparameter analysis of heterozygous cell populations on a cell-by-cell basis. In an aspect, immunophenotyping can be used to detect specific antigens and/or markers that are expressed on various cells. Immunophenotyping uses fluorochrome-tagged monoclonal antibodies to analyze heterogenous populations of cells. Fluorescence in situ hybridization (FISH) can be used to characterize structural chromosome abnormalities and identify chromosomes of uncertain origin. Cytogenetics can be used to analyze the chromosomes during metaphase as it describes the number of chromosomes and their appearance, thereby identifying chromosomal abnormalities. PCR can be used to amplify DNA and/or RNA for analyses. Gene expression profiles can be used to measure the activity of genes.

In an aspect, a disclosed method can further comprise decreasing and/or reducing one or more symptoms related to and/or associated with the subject's hematological cancer. In an aspect, decreasing and/or reducing one or more symptoms related to and/or associated with the subject's hematological cancer can a 10%, 20%, 30%, 40%, 50%, 60%, 70%, 80%, 90%, 100%, or any amount of decrease and/or reduction in the risk of and/or actual metastasis of cancer cells when compared to a control subject (such as, for example, a subject that has not received a disclosed treatment (e.g., a disclosed chimeric fusion protein or a pharmaceutical formulation thereof). In an aspect, preventing or inhibiting metastasis of cancer cells can comprise a 10-20%, 20-30%, 30-40%, 40-50%, 50-60%, 60-70%, 70-80%, 80-90%, or 90-100% or any amount of decrease and/or reduction in the risk of and/or actual metastasis of cancer cells when compared to a control subject (such as, for example, a subject that has not received a disclosed treatment (e.g., a disclosed chimeric fusion protein or a pharmaceutical formulation thereof).

In an aspect, a disclosed method of treating a hematological cancer can comprise stimulating an effector cell mediated immune modulator response to PS-expressing tumor cells. In an aspect, a disclosed method of treating a hematological cancer can induce a tumor reducing immune response. In an aspect, a disclosed method of treating a hematological cancer can induce phagocytosis of cancer cells in the subject. In an aspect, a disclosed method of treating a hematological cancer can cross-prime an anti-tumor T cell response. In an aspect, a disclosed method of treating a hematological cancer can induce a tumor eliminating immune response. In an aspect, a disclosed method of treating a hematological cancer can effect cell death of PS-expression cells. In an aspect, a disclosed method of treating a hematological cancer can comprise improving the efficacy of one or more anti-cancer therapies and/or treatments.

In an aspect, a disclosed method of treating a hematological cancer can comprise generating a disclosed chimeric fusion protein or pharmaceutical formulation thereof.

In an aspect, a disclosed method of treating a hematological cancer can comprise generating a disclosed chimeric fusion protein using one or more disclosed vectors and/or one or more disclosed plasmids and/or one or most disclosed cells and/or any combination thereof.

In an aspect, a disclosed method of treating a hematological cancer can comprise validating the efficacy and/or toxicity of disclosed chimeric fusion protein or pharmaceutical formulation thereof. In an aspect, validating the efficacy and/or toxicity of a disclosed chimeric fusion protein or pharmaceutical formulation thereof can comprise using an in vivo model and/or in vitro model.

In an aspect, a disclosed method of treating a hematological cancer can further comprise administering genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof one or more times, administering one or more anti-cancer therapies one or more times, or administering any combination thereof one or more time.

In an aspect, a disclosed method of treating a hematological cancer can improve and/or extend the survivability of the subject, can improve a subject's quality of life, can increase and/or prolong a subject's life span, or any combination thereof. In an aspect, a disclosed method of treating a hematological cancer can stimulate an effector cell mediated immune modulator response to PS-expressing tumor cells. In an aspect, a disclosed method of treating a hematological cancer can induce a tumor reducing immune response. In an aspect, a disclosed method of treating a hematological cancer can induce phagocytosis of cancer cells in the subject. In an aspect, a disclosed method of treating a hematological cancer can cross-prime an anti-tumor T cell response. In an aspect, a disclosed method of treating a hematological cancer can induce a tumor eliminating immune response. In an aspect, a disclosed pharmaceutical formulation can treat cancer.

In an aspect of a disclosed method of treating a hematological cancer, a disclosed chimeric fusion protein can be replaced by a disclosed vector. In an aspect of a disclosed method of treating a hematological cancer, a disclosed chimeric fusion protein can be replaced by a disclosed nucleic acid molecule. In an aspect of a disclosed method of treating a hematological cancer, a disclosed chimeric fusion protein can be replaced by a disclosed plasmid. In an aspect of a disclosed method of treating a hematological cancer, a disclosed chimeric fusion protein can be replaced by a disclosed antibody drug conjugate. In an aspect of a disclosed method of treating a hematological cancer, a disclosed chimeric fusion protein can be replaced by a disclosed pharmaceutical formulation.

### F. Methods of Improving the Efficacy of Cancer Therapy

Disclosed herein is a method of improving the efficacy of cancer therapy, the method comprising treating a subject in need thereof by administering to the subject in need thereof a therapeutically effective amount of one or more cells transduced with a disclosed chimeric fusion protein or a pharmaceutical formulation thereof. Disclosed herein is a method of improving the efficacy of cancer therapy, the method comprising treating a subject in need thereof by administering to the subject in need thereof a therapeutically effective amount of a disclosed chimeric fusion protein or a pharmaceutical formulation thereof.

Disclosed herein is a method of improving the efficacy of cancer therapy, the method comprising treating a subject in need thereof by administering to the subject in need thereof a therapeutically effective amount of a disclosed chimeric fusion protein targeting phosphatidylserine (PS) or a pharmaceutical formulation thereof. Disclosed herein is a method of improving the efficacy of cancer therapy, the method comprising treating a subject in need thereof by administering to the subject in need thereof a therapeutically effective amount of a disclosed chimeric fusion protein targeting phosphatidylserine-expressing tumor cells or a pharmaceutical formulation thereof.

In an aspect, wherein the disclosed chimeric fusion protein targets phosphatidylserine (PS)-expressing cancer cells. In an aspect, wherein the disclosed chimeric fusion protein targets phosphatidylserine (PS)-expressing hematological cancer cells. In an aspect, disclosed PS-expressing cancer cells can be lymphoma cells, leukemia cells, and multiple myeloma cells. In an aspect, disclosed PS-expressing cancer cells can be acute myeloid leukemia (AML) or myelodysplastic syndrome. In an aspect, disclosed PS-expressing cancer cells can be blood borne.

In an aspect, a subject can be treatment-naïve. In an aspect, a subject has received one or more treatments prior to the administering of a disclosed chimeric fusion protein or pharmaceutical formulation thereof. In an aspect, one or more treatments can comprise one or more anti-cancer therapies and/or treatments.

In an aspect, a subject can have, be diagnosed with, or be suspected of having one or more cancers. In an aspect, a disclosed a method of improving the efficacy of cancer therapy can further comprise diagnosing the subject as have cancer or cancerous cells. In an aspect, a disclosed a method of improving the efficacy of cancer therapy can further comprise diagnosing the subject as having a hematological cancer or cancerous hematological cells.

In an aspect, a disclosed cancer can be a hematologic cancer. In an aspect, a disclosed hematological cancer can comprise lymphoma, myeloma, leukemia, or any combination thereof.

In an aspect, a subject can have, be diagnosed with, or be suspected of having one or more cancers. In an aspect, a subject can have, be diagnosed with, or be suspected of having a hematological cancer and one or more other cancers. In an aspect, other cancers can comprise ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

In an aspect, a disclosed method of improving the efficacy of cancer therapy can further comprise collecting one or more blood and/or biological samples from a subject at the same time or at different times. For example, in an aspect, a blood sample and/or a biological sample can be collected from a subject at a pre-determined interval. In an aspect, a pre-determined interval can be once a week, once every 2 weeks, once every 3 weeks, once every 4 weeks, once every 5 weeks, once every 6 weeks, once every 7 weeks, once every 8 weeks, or at a longer interval. In an aspect, a pre-determined interval can be once a month, once every 2 months, once every 3 months, once every 5 months, once every 5 months, once every 6 months, or at a longer interval. In an aspect, a blood sample and/or a biological sample can be collected from a subject prior to treatment, during treatment, after treatment, or any combination thereof. In an aspect, a blood and/or a biological sample can be collected from a subject at any time deemed medically and/or clinically appropriate by the skilled clinician.

In an aspect, a therapeutically effective amount or effective dose or effective amount or therapeutically effective dosage of a disclosed chimeric fusion protein or pharmaceutical formulation thereof can be any amount that, when used alone or in combination with another therapeutic agent, can attack and destroy PS-expressing tumor cells. In an aspect, a therapeutically effective amount or effective dose or effective amount or therapeutically effective dosage of a disclosed chimeric fusion protein or pharmaceutical formulation thereof can be any amount that, when used alone or in combination with another therapeutic agent, can attack and destroy PS-expressing tumor cells. In an aspect, a therapeutically effective amount or effective dose or effective amount or therapeutically effective dosage of a disclosed chimeric fusion protein or pharmaceutical formulation thereof can be any amount that can enhance the success rate of one or more conventional cytotoxic cancer therapies against PS-expressing tumor cells.

In an aspect, a disclosed therapeutically effective amount or effective dose of a disclosed chimeric fusion protein or pharmaceutical formulation thereof can be about 0.01 μg/kg to about 10 mg/kg per subject.

In an aspect, a therapeutically effective amount or effective dose or effective amount or therapeutically effective dosage of a disclosed chimeric fusion protein or pharmaceutical formulation thereof can protect a subject against the onset of a disease and/or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

In an aspect, administering a disclosed chimeric fusion protein or pharmaceutical formulation thereof can comprise systemic or direct administration. In an aspect, administering can comprise oral administration, intravenous administration, intratumoral administration, intraperitoneal administration, intracranial administration, subcutaneous administration, intradermal administration, intrathecal administration, or any combination thereof. In an aspect, administering a disclosed chimeric fusion protein or pharmaceutical formulation thereof can be administered by any method of administration disclosed herein. In an aspect, a disclosed chimeric fusion protein or pharmaceutical formulation thereof can be administered via multiple routes either concurrently or sequentially.

For example, in an aspect, a disclosed chimeric fusion protein or pharmaceutical formulation thereof can be first administered intratumorally and then be administered intravenously. In an aspect, a disclosed chimeric fusion protein or pharmaceutical formulation thereof can be first administered intratumorally and then be administered orally. A skilled clinician can determine the best route of administration for a subject at a given time.

In an aspect, a disclosed method can comprise repeating the administering of a disclosed chimeric fusion protein or pharmaceutical formulation thereof.

In an aspect, a disclosed method of improving the efficacy of cancer therapy can comprise protecting the subject from metastasis. In an aspect, a disclosed method of improving the efficacy of cancer therapy can comprise reducing the risk of developing metastasis. In an aspect, a disclosed method of improving the efficacy of cancer therapy can comprise preventing or inhibiting metastasis.

In an aspect, a disclosed method of improving the efficacy of cancer therapy can comprise monitoring the subject for adverse effects. In an aspect, in the absence of adverse effects, a disclosed method can comprise continuing to treat the subject. In an aspect, continuing to treat the subject can comprise continuing to administer to the subject a disclosed chimeric fusion protein or pharmaceutical formulation thereof. In an aspect, in the presence of adverse effects, a disclosed method can comprise modifying one or more steps of the method. In an aspect, modifying one or more steps of a disclosed method can comprise modifying the administering step. In an aspect, modifying the administering step can comprise changing the amount of a disclosed chimeric fusion protein or pharmaceutical formulation thereof administered to the subject, changing the frequency of administration of a disclosed chimeric fusion protein or pharmaceutical formulation thereof, changing the duration of administration of a disclosed chimeric fusion protein or pharmaceutical formulation thereof, changing the route of administration of a disclosed chimeric fusion protein or pharmaceutical formulation thereof, or any combination thereof.

In an aspect, a disclosed method of improving the efficacy of cancer therapy can further comprise administering to the subject an immune checkpoint inhibitor (e.g., an anti-PD1 molecule). In an aspect, a disclosed anti-PD1 molecule can comprise an anti-PD1 antibody, an anti-PDL1 antibody, or any combination thereof. In an aspect, a disclosed anti-PD1 antibody can comprise a monoclonal antibody, a humanized monoclonal antibody, or a fragment thereof. In an aspect, a disclosed anti-PD1 antibody can comprise a polyclonal antibody, a humanized polyclonal antibody, or a fragment thereof. In an aspect, a disclosed anti-PD1 antibody can comprise any antibody or antibody fragment that specifically recognizes PD1. In an aspect, a disclosed anti-PDL1 antibody can comprise a monoclonal antibody, a humanized monoclonal antibody, or a fragment thereof. In an aspect, a disclosed anti-PDL1 antibody can comprise a polyclonal antibody, a humanized polyclonal antibody, or a fragment thereof. In an aspect, a disclosed anti-PDL1 antibody can comprise any antibody or antibody fragment that specifically recognizes PDL1. Antibodies and methods of preparing antibodies are known in the art. Similarly, recombinant antibodies and methods of preparing recombinant antibodies are known in the art.

In an aspect, a disclosed method of improving the efficacy of cancer therapy can further comprise repeating the administering of the anti-PD1 molecule. In an aspect, a disclosed anti-PD1 molecule can be administered prior to, concurrent with, or after the administration of the interfering molecule.

In an aspect of a disclosed method of improving the efficacy of cancer therapy, administering a disclosed anti-PD1 molecule can comprise systemic or direct administration. In an aspect, administering a disclosed anti-PD1 molecule can comprise intravenous administration, intratumoral administration, intraperitoneal administration, or any combination thereof. In an aspect, administering a disclosed can be administered by any method of administration disclosed herein. In an aspect, a disclosed anti-PD1 molecule can be administered via multiple routes either concurrently or sequentially. For example, in an aspect, a disclosed anti-PD1 molecule can be first administered intratumorally and then be administered intravenously. In an aspect, administering a disclosed anti-PD1 molecule can be first administered intratumorally and then be administered orally. A skilled clinician can determine the best route of administration for a subject at a given time.

For example, in an aspect, a disclosed anti-PD1 molecule can be administered about 3 months, about 2 months, or about 1 month prior to the administering of a disclosed chimeric fusion protein or pharmaceutical formulation thereof. In an aspect, a disclosed anti-PD1 molecule can be administered about 8 weeks, about 7 weeks, about 6 weeks, about 5 weeks, about 4 weeks, about 3 weeks, about 2 weeks, or about 1 week prior to the administering of a disclosed chimeric fusion protein or pharmaceutical formulation thereof. In an aspect, a disclosed anti-PD1 molecule can be administered about 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day prior to the administering of a disclosed chimeric fusion protein or pharmaceutical formulation thereof. In an aspect, a disclosed anti-PD1 molecule can be administered about 24, 23, 22, 21, 20, 19, 18, 17, 16, 15, 14, 13, 12, 11, 10, 9, 8, 7, 6, 5, 4, 3, 2, or 1 hours prior to the administering of a disclosed chimeric fusion protein or pharmaceutical formulation thereof. In an aspect, a disclosed method of improving the efficacy of cancer therapy can further comprise repeating the administering of a disclosed anti-PD1 molecule. In an aspect, a disclosed anti-PD1 molecule can be administered prior to, concurrent with, or after the administration of the disclosed chimeric fusion protein.

In an aspect, a disclosed method of improving the efficacy of cancer therapy can comprise administering to the subject one or more additional anti-cancer therapies. Anti-cancer therapies are known to the art. In an aspect, a disclosed anti-cancer therapy can comprise endocrine therapy, radiotherapy, hormone therapy, gene therapy, thermal therapy, ultrasound therapy, or any combination thereof. In an aspect, a disclosed anti-cancer therapy can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof. In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), axitinib (Inlyta), bevacizumab (Avastin), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, irinotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof. In an aspect, a disclosed method of improving the efficacy of cancer therapy can further comprise repeating the administering of a disclosed chemotherapeutic agent. In an aspect, a disclosed chemotherapeutic agent can be administered prior to, concurrent with, or after the administration of the disclosed chimeric fusion protein.

In an aspect, a disclosed method of improving the efficacy of cancer therapy can comprise administering to the subject an anti-chemokine therapy. In an aspect, a disclosed anti-chemokine therapy can comprise one or more antibodies against CCL1, CCL2, CCL4, CCL17, CCL19, CCL21, CCL22, CCL25, CXCL9, CXCL10, CXCL11, CXCL12, CXCL13, CCR2, CCR5, CCR7, CCR8, CCR9, CXCR3, CXCR4, CXCR5, CX3CL1, CX3CR1, or any combination thereof. In an aspect, a disclosed method of improving the efficacy of cancer therapy can further comprise repeating the administering of a disclosed anti-chemokine therapy. In an aspect, a disclosed anti-chemokine therapy can be administered prior to, concurrent with, or after the administration of the disclosed chimeric fusion protein.

In an aspect, a disclosed method of improving the efficacy of cancer therapy can comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. CRISPR/Cas9 systems and methods are known to the art. In an aspect, a disclosed method of improving the efficacy of cancer therapy can further comprise repeating the administering of a disclosed oligonucleotide therapeutic agent. In an aspect, a disclosed oligonucleotide therapeutic agent can be administered prior to, concurrent with, or after the administration of the disclosed chimeric fusion protein.

In an aspect, a disclosed method of improving the efficacy of cancer therapy can further comprise preventing or inhibiting metastasis of cancer cells. In an aspect, a disclosed method can comprise a 10%, 20%, 30%, 40%, 50%, 60%, 70%, 80%, 90%, 100%, or any amount of decrease and/or reduction in the risk of and/or actual metastasis of cancer cells when compared to a control subject (such as, for example, a subject that has not received a disclosed treatment (e.g., a disclosed chimeric fusion protein or a pharmaceutical formulation thereof)). In an aspect, preventing or inhibiting metastasis of cancer cells can comprise a 10-20%, 20-30%, 30-40%, 40-50%, 50-60%, 60-70%, 70-80%, 80-90%, or 90-100% or any amount of decrease and/or reduction in the risk of and/or actual metastasis of cancer cells when compared to a control subject (such as, for example, a subject that has not received a disclosed treatment (e.g., a disclosed chimeric fusion protein or a pharmaceutical formulation thereof).

In an aspect, a disclosed method of improving the efficacy of cancer therapy can comprise surgically resecting the tumor and/or cancer cells from the subject. In an aspect, following resecting the tumor and/or cancer cells from the subject, a disclosed method of improving the efficacy of cancer therapy can comprise continuing to administer to the subject a therapeutically effective amount of a disclosed chimeric fusion protein or a pharmaceutical formulation thereof and continuing to administer to the subject a therapeutically effective amount of an anti-PD1 molecule, a disclosed anti-chemokine therapy, a disclosed chemotherapeutic agent, any disclosed therapeutic agent, or any combination thereof.

In an aspect, a disclosed method of improving the efficacy of cancer therapy can further comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed method of improving the efficacy of cancer therapy can restore one or more aspects of cellular homeostasis and/or cellular functionality and/or metabolic dysregulation in a subject, such as, for example, a subject having cancer or cancerous cells. In an aspect, a disclosed chimeric fusion protein or pharmaceutical formulation thereof can restore one or more aspects of cellular homeostasis and/or cellular functionality and/or metabolic dysregulation in a subject having cancer. In an aspect, metabolic dysregulation can be associated with cancer or cancerous cells. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cancer; (vii) reversing, inhibiting, preventing, stabilizing, and/or slowing the rate of progression of a cancer, or (viii) any combination thereof. In an aspect, restoring one or more aspects of cellular homeostasis can comprise improving, enhancing, restoring, and/or preserving one or more aspects of cellular structural and/or functional integrity. In an aspect, restoration can be a partial or incomplete restoration. In an aspect, restoration can be complete or near complete restoration such that the level of expression, activity, and/or functionality is similar to that of a wild-type or control level. In an aspect, restoring one or more aspects of cellular homeostasis and/or cellular functionality can comprise preventing or inhibiting metastasis of cancer cells in the subject.

In an aspect of a disclosed a method of improving the efficacy of cancer therapy, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are discussed supra.

In an aspect of a disclosed method of improving the efficacy of cancer therapy, techniques to monitor, measure, and/or assess the status of a subject's hematological disease and/or disorder and/or cancer can comprise qualitative (or subjective) means as well as quantitative (or objective) means. In an aspect, microscopy can be used for the visualization of cells to determine morphology and staining characteristics. In an aspect, immunohistochemistry can be used to identify specific molecules in different kinds of cells and/or tissues. The tissue is treated with antibodies that bind to the specific molecule. These are made visible under a microscope by using a color reaction, a radioisotope, or a fluorescent dye. In an aspect, flow cytometry can be used to sort and collect cells for additional analyses as flow cytometer is capable of rapid, quantitative, multiparameter analysis of heterozygous cell populations on a cell-by-cell basis. In an aspect, immunophenotyping can be used to detect specific antigens and/or markers that are expressed on various cells. Immunophenotyping uses fluorochrome-tagged monoclonal antibodies to analyze heterogenous populations of cells. Fluorescence in situ hybridization (FISH) can be used to characterize structural chromosome abnormalities and identify chromosomes of uncertain origin. Cytogenetics can be used to analyze the chromosomes during metaphase as it describes the number of chromosomes and their appearance, thereby identifying chromosomal abnormalities. PCR can be used to amplify DNA and/or RNA for analyses. Gene expression profiles can be used to measure the activity of genes.

In an aspect, a disclosed a method of improving the efficacy of cancer therapy can further comprise decreasing and/or reducing one or more symptoms related to and/or associated with the subject's hematological cancer. In an aspect, decreasing and/or reducing one or more symptoms related to and/or associated with the subject's hematological cancer can a 10%, 20%, 30%, 40%, 50%, 60%, 70%, 80%, 90%, 100%, or any amount of decrease and/or reduction in the risk of and/or actual metastasis of cancer cells when compared to a control subject (such as, for example, a subject that has not received a disclosed treatment (e.g., a disclosed chimeric fusion protein or a pharmaceutical formulation thereof). In an aspect, preventing or inhibiting metastasis of cancer cells can comprise a 10-20%, 20-30%, 30-40%, 40-50%, 50-60%, 60-70%, 70-80%, 80-90%, or 90-100% or any amount of decrease and/or reduction in the risk of and/or actual metastasis of cancer cells when compared to a control subject (such as, for example, a subject that has not received a disclosed treatment (e.g., a disclosed chimeric fusion protein or a pharmaceutical formulation thereof).

In an aspect, a disclosed method of improving the efficacy of cancer therapy can comprise stimulating an effector cell mediated immune modulator response to PS-expressing tumor cells. In an aspect, a disclosed method of improving the efficacy of cancer therapy can induce a tumor reducing immune response. In an aspect, a disclosed method of improving the efficacy of cancer therapy can induce phagocytosis of cancer cells in the subject. In an aspect, a disclosed method of improving the efficacy of cancer therapy cross-prime an anti-tumor T cell response. In an aspect, a disclosed method of improving the efficacy of cancer therapy can induce a tumor eliminating immune response. In an aspect, a disclosed method of improving the efficacy of cancer therapy can effect cell death of PS-expression cells. In an aspect, a disclosed method of improving the efficacy of cancer therapy can comprise improving the efficacy of one or more anti-cancer therapies and/or treatments.

In an aspect, a disclosed method of improving the efficacy of cancer therapy can comprise generating a disclosed chimeric fusion protein or pharmaceutical formulation thereof.

In an aspect, a disclosed method of improving the efficacy of cancer therapy can comprise generating a disclosed chimeric fusion protein using one or more disclosed vectors and/or one or more disclosed plasmids and/or one or most disclosed cells and/or any combination thereof.

In an aspect, a disclosed method of improving the efficacy of cancer therapy can comprise validating the efficacy and/or toxicity of disclosed chimeric fusion protein or pharmaceutical formulation thereof. In an aspect, validating the efficacy and/or toxicity of a disclosed chimeric fusion protein or pharmaceutical formulation thereof can comprise using an in vivo model and/or in vitro model.

In an aspect, a disclosed method of improving the efficacy of cancer therapy can further comprise administering genetically modified cells expressing a chimeric antigen receptor targeting phosphatidylserine (PS) or a pharmaceutical formulation thereof or engineered T cells or NK cells or macrophages expressing a chimeric antigen receptor targeting phosphatidylserine (PS) or a pharmaceutical formulation thereof one or more times, administering an anti-PD1 molecule one or more times, administering one or more anti-cancer therapies one or more times, or administering any combination thereof one or more time.

In an aspect, a disclosed method of treating a hematological cancer can improve and/or extend the survivability of the subject, can improve a subject's quality of life, can increase and/or prolong a subject's life span, or any combination thereof. In an aspect, a disclosed method of improving the efficacy of cancer therapy can stimulate an effector cell mediated immune modulator response to PS-expressing tumor cells. In an aspect, a disclosed method of improving the efficacy of cancer therapy can induce a tumor reducing immune response. In an aspect, a disclosed method of improving the efficacy of cancer therapy can induce phagocytosis of cancer cells in the subject. In an aspect, a disclosed method of improving the efficacy of cancer therapy can cross-prime an anti-tumor T cell response. In an aspect, a disclosed method of improving the efficacy of cancer therapy can induce a tumor eliminating immune response. In an aspect, a disclosed method of improving the efficacy of cancer therapy formulation can treat cancer.

In an aspect of a disclosed method of improving the efficacy of cancer therapy, a disclosed chimeric fusion protein can be replaced by a disclosed vector. In an aspect of a disclosed method of improving the efficacy of cancer therapy, a disclosed chimeric fusion protein can be replaced by a disclosed nucleic acid molecule. In an aspect of a disclosed method of improving the efficacy of cancer therapy, a disclosed chimeric fusion protein can be replaced by a disclosed plasmid. In an aspect of a disclosed method of improving the efficacy of cancer therapy, a disclosed chimeric fusion protein can be replaced by a disclosed antibody drug conjugate. In an aspect of a disclosed method of improving the efficacy of cancer therapy, a disclosed chimeric fusion protein can be replaced by a disclosed pharmaceutical formulation.

### G. Kits

Disclosed herein is a kit comprising one or more of a disclosed chimeric fusion protein, a disclosed pharmaceutical formulation, a disclosed isolated nucleic acid molecule, a disclosed vector, a disclosed host cell, or any combination thereof. Disclosed herein is a kit comprising one or more disclosed chimeric fusion proteins, one or more disclosed pharmaceutical formulations, one or more disclosed isolated nucleic acid molecule, one or more disclosed vectors, one or more disclosed host cells, or any combination thereof.

In an aspect, a disclosed kit can comprise one or more additional active agents and/or therapeutic agents. In an aspect, the one or more agents can treat, prevent, inhibit, and/or ameliorate one or more comorbidities in a subject. In an aspect, one or more active agents can treat, inhibit, prevent, and/or ameliorate cellular and/or metabolic complications related to cancer or cancer cells or cancerous cells.

In an aspect, a disclosed kit can comprise a disclosed chimeric fusion protein (e.g., SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:99, SEQ ID NO:100, SEQ ID NO:101, SEQ ID NO:106, or a fragment thereof).

In an aspect, a disclosed kit can comprise a disclosed bispecific chimeric fusion protein as set forth FIG. 5A-FIG. 5H.

In an aspect, a disclosed kit can comprise at least two components constituting the kit. Together, the components constitute a functional unit for a given purpose (such as, for example, treating a subject diagnosed with or suspected of having a disease or disorder such as cancer). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a disclosed kit for use in a disclosed method can comprise one or more containers holding a disclosed chimeric fusion protein, a disclosed pharmaceutical formulation, a disclosed isolated nucleic acid molecule, a disclosed vector, a disclosed host cell, or any combination thereof, and a label or package insert with instructions for use. In an aspect, suitable containers include, for example, bottles, vials, syringes, blister pack, etc. The containers can be formed from a variety of materials such as glass or plastic. The container can hold a disclosed chimeric fusion protein, a disclosed pharmaceutical formulation, a disclosed isolated nucleic acid molecule, a disclosed vector, a disclosed host cell, or any combination thereof, and can have a sterile access port (for example the container may be an intravenous solution bag or a vial having a stopper pierceable by a hypodermic injection needle). The label or package insert can indicate a disclosed chimeric fusion protein, a disclosed pharmaceutical formulation, a disclosed isolated nucleic acid molecule, a disclosed vector, a disclosed host cell, or any combination thereof can be used for treating, preventing, inhibiting, and/or ameliorating a disease or disorder or complications and/or symptoms associated with a disease or disorder such as cancer or metastatic cancer. A kit can comprise additional components necessary for administration such as, for example, other buffers, diluents, filters, needles, and syringes.

In an aspect, a disclosed kit can be used to preventing or inhibiting metastasis of cancer cells. In an aspect, a disclosed kit can be used to risk of developing metastases. In an aspect, a disclosed kit can be used to validate the efficacy and/or toxicity of a disclosed chimeric fusion protein, a disclosed pharmaceutical formulation, a disclosed isolated nucleic acid molecule, a disclosed vector, a disclosed host cell, or any combination thereof.

In an aspect, a disclosed kit can be used to improve and/or extend the survivability of the subject, can improve a subject's quality of life, can increase and/or prolong a subject's life span, or any combination thereof. In an aspect, a disclosed kit can be used to can stimulate an effector cell mediated immune modulator response to PS-expressing tumor cells. In an aspect, a disclosed kit can be used to induce a tumor reducing immune response. In an aspect, a disclosed kit can be used to induce phagocytosis of cancer cells in the subject. In an aspect, a disclosed kit can be used to cross-prime an anti-tumor T cell response. In an aspect, a disclosed kit can be used to induce a tumor eliminating immune response. In an aspect, a disclosed kit can be used to treat cancer.

### H. Miscellaneous

Disclosed herein is a chimeric fusion protein, the chimeric fusion protein comprising phosphatidylserine (PS) binding domain, optionally a linker region, and an immunostimulatory domain. In an aspect of a disclosed chimeric fusion protein, a phosphatidylserine (PS)-binding domain comprises a naturally existing protein. In an aspect, a disclosed naturally exiting protein comprises (1) Human annexin A1 or its PS-binding core domain (aa41-346); (2) Human annexin A2; (3) Human annexin A3; (4) Human annexin A4; (5) Human annexin A5; (6) Human annexin A6; (7) Human annexin A7; (8) Human annexin A8; (9) Human annexin A8L1: (10) Human annexin A8L2; (11) Human annexin A9; (12) Human annexin A10; (13) Human annexin A11; (14) Human annexin A13; (15) Extracellular domain of human BAI1 (brain angiogenesis inhibitor 1); (16) Human beta 2-glycoprotein I; (17) Human factor II; (18) Human factor VII; (19) Huma factor IX; (20) Human factor X; (21) Human prothrombin; (22) Human growth arrest specific 6 (GAS6); (23) Human MFG-E8 (lactaherin); (24) Extracellular domain of human RAGE; (25) Protein S; (26) Extracellular domain of STABILIN 1 & 2; (27) Extracellular domain of TIM1; (28) Extracellular domain of Tim3; (29) Extracellular domain of TIM4; (30) Phosphoatidylserine receptor (PSR); (31) Human PKCa C2 domain (aa 157-288); (32) RAGE (receptor for advanced glycation end products); (33) Human synaptotagmin (Syt1) C2A domain (aa141-266); (34) Stabilin-1; (35) Stabilin-2; or any combinations thereof. In an aspect, a disclosed PS-binding domain comprises the single chain variable region (scFv) of known anti-PS antibodies. In an aspect, a disclosed anti-PS antibody comprises (1) Bavituximab; (2) PS binding antibody PGN632 (as published by Moody et al, JEM, 2010, 207:763-776); (3) PS binding antibody P1 (as published by Moody et al, JEM, 2010, 207:763-776); (4) PS binding antibody IS4 (as published by Moody et al, JEM, 2010, 207:763-776); (5) PS binding antibody CL1 (as published by Moody et al, JEM, 2010, 207:763-776); or any combinations thereof.

In an aspect, a disclosed chimeric fusion protein comprises an optional linker. In an aspect, a disclosed linker comprises (1) a flexile linker; (2) a rigid linker; (3) a Whitlow Linker; or any combinations thereof. In an aspect, a disclosed chimeric fusion protein comprises a Flexile linker.

In an aspect, a disclosed flexile linker comprises, but is not limited to, (GGGGS)n, where n=1-8; and (G)n, where n=1-8. In an aspect, a disclosed linker comprises a rigid linker. In an aspect, a disclosed rigid linker comprises, but is not limited to, (EAAAK)n, where n=1-8. In an aspect, a disclosed linker comprises a Whitlow linker. In an aspect, a disclosed Whitlow linker comprises (GSTSGSGKSSEGKG)n, or (GSTSGSGKPGSGEGSTKG)n, where n=1-8.

In an aspect, a disclosed immunostimulatory domain comprises the Fc domain of human IgG or a single chain antibody (scFv) to a human CD3 protein. In an aspect, a disclosed immunostimulatory domain comprises the Fc domain of human IgG selected from the group consisting of IgG1, IgG2, IgG3, IgG4 and combinations thereof. In an aspect, a disclosed immunostimulatory domain of a single chain antibody (scFv) to a human CD3 protein includes, but not limited to OKT3, humanized OKT3, or other published anti-human CD3 antibodies. In an aspect, an anti-CD3 antibody can comprise a OKT3 or humanized OKT3 antibody. In an aspect, an anti-CD3 anti-body can comprise OKT3 (muromab), ChAglyCD3 (otelixizumab), hOKT3γ1 (teplizumab), nuvion (visilizumab), NI-0401 (foralumab), a UCHT1 or humanized UCHT1 antibody, tepilizumab, TRX4 (otlixizumab), or any combination thereof. In an aspect, a dis-closed immunostimulatory domain can comprise one or more anti-CD3 antibodies.

Disclosed herein is a chimeric fusion protein, the chimeric fusion protein comprising phosphatidylserine (PS) binding domain comprising human annexin, optionally a linker region, and an immunostimulatory domain comprising the Fc domain of IgG1. In an aspect, a disclosed Annexin V-IgG1Fc fusion protein is further conjugated at the Fc region with a small molecule compound to enhance its cancer cell killing or immune-stimulating activities. In an aspect, a disclosed small molecule is selected from the group consisting of (1) Auristatins; (2) Maytasinoids; (3) Calichemicins; (4) Camptothecins; (5) STING agonists; (6) SN38 and combinations thereof. In an aspect, a disclosed PS binding domain-Fc fusion protein is further fused to an additional immune-stimulatory protein. In an aspect, a disclosed additional immune-stimulatory protein comprises a subdomain of human SIRPα(IgV domain) protein that can bind CD47. In an aspect, a disclosed additional immune-stimulatory protein comprises the extracellular domain of the NKG2D protein. In an aspect, a disclosed fusion protein comprises annexin V fused with a single-chain antibody (scFv) against CD3. In an aspect, a disclosed PS-binding domain fused with an scFv against CD3 is fused with an Fc domain, duplicated through a Fc hinge region, or fused with an Fc domain and paired with a Fc domain only.

Disclosed herein is a chimeric fusion protein comprising a heterozygous dimer of Annexin V-Fc:αCD3 scFv-Fc. In an aspect, a disclosed Annexin V is human annexin V or versions of Annexin V comprises at least 80% similarity to human Annexin V.

Disclosed herein is a pharmaceutical composition, the pharmaceutical composition comprising, a chimeric fusion protein as in any of the preceding claims and a pharmaceutically acceptable excipient, diluent, and/or carrier.

Disclosed herein is a method of treating hematological cancers by use of one or more chimeric fusion proteins as in any of the preceding claims such that the hematological cancer is treated in the subject. In an aspect, a disclosed method comprises further administering one or more additional therapeutic agents. In an aspect, a disclosed one or more additional therapeutic agents is selected from the group consisting of chemotherapy or radiotherapy to target those cancer cells that express phosphatidylserine on their surface but remain alive and escape cytotoxic therapy. In an aspect, disclosed hematological cancers are selected from the group consisting of lymphoma, leukemia, and multiple myeloma. In an aspect, the hematological cancer comprises acute myeloid leukemia (AML) or myelodysplastic syndrome. In an aspect, disclosed chimeric fusion proteins are administered to the subject by a method selected from the group consisting of: (i) Intravenous (IV); (ii) Intracranial; (iii) Intraperitoneal; (iv) Subcutaneously; (v) Intradermal; (vi) Intrathecal; and combinations thereof. In an aspect, disclosed chimeric fusion proteins are delivered by use of a DNA or RNA construct encoding the chimeric protein. In an aspect, disclosed chimeric fusion proteins are delivered by use of a DNA construct. In an aspect, a disclosed DNA construct is based on a vector/virus selected from the group consisting of adenovirus, lentivirus, AAV, HSV, plasmid vectors and combinations thereof encoding the chimeric protein. In an aspect, disclosed chimeric fusion proteins are delivered by use of an RNA construct. In an aspect, a disclosed RNA construct comprises an RNA that is synthesized in vivo through in vitro transcription of plasmid vectors.

| SEQ ID | | |
|---|---|---|
| NO. | Description of Sequence | Type |
| 1 | ANXA1 | human protein |
| 2 | ANXA1 (PS Binding Domain of 41-346) | human protein |
| 3 | ANXA2 | human protein |
| 4 | ANXA3 | human protein |
| 5 | ANXA4 | human protein |
| 6 | ANXA5 | human protein |
| 7 | ANXA6 | human protein |
| 8 | ANXA7 | human protein |
| 9 | ANXA8 | human protein |
| 10 | ANXA8L1 | human protein |
| 11 | ANXA9 | human protein |
| 12 | ANXA10 | human protein |
| 13 | ANXA11 | human protein |
| 14 | ANXA13 | human protein |
| 15 | ADGRB1 (BAI1) | human protein |
| 16 | ADGRB1 (BAI) (extracellular domain 31-948) | human protein |
| 17 | Apolipoprotein H (Beta-2-Glycoprotein I) | human protein |
| 18 | Coagulation Factor II | human protein |
| 19 | Coagulation Factor VII | human protein |
| 20 | Coagulation Factor IX | human protein |
| 21 | Coagulation Factor X | human protein |
| 22 | Growth Arrest Specific 6 | human protein |
| 23 | Milk Fat Globule EGF and Factor V/VIII Domain Containing | human protein |
| | (MFG-E8) (Lactadherin) | |
| 24 | AGER/RAGE | human protein |
| 25 | AGER/RAGE (ExD 23-342) | human protein |
| 26 | Protein S | human protein |
| 27 | Stabilin 1 | human protein |
| 28 | Stabilin 1 (ExD 26-2478) | human protein |
| 29 | Stabilin 2 | human protein |
| 30 | Stabilin 2 (ExD 20-2458) | human protein |
| 31 | Hepatitis A Virus Cellular Receptor 1/TIM1 | human protein |
| 32 | Hepatitis A Virus Cellular Receptor 1/TIM1 (ExD 21-295) | human protein |
| 33 | Hepatitis A Virus Cellular Receptor 2/TIM3 | human protein |
| 34 | Hepatitis A Virus Cellular Receptor 2/TIM3 (Ex 22-202) | human protein |
| 35 | T Cell Immunoglobulin And Mucin Domain Containing | human protein |
| | (TIMD4) | |
| 36 | T Cell Immunoglobulin And Mucin Domain Containing/ | human protein |
| | TIMD4 (ECD 25-314) | |
| 37 | Protein Kinase C Alpha | human protein |
| 38 | Protein Kinase C Alpha (C2 domain 158-275) | human protein |
| 39 | Synaptotagmin (SYT1) | human protein |
| 40 | Synaptotagmin (C2A domain 142-261) | human protein |
| 41 | Jumonji Domain Containing 6, Arginine Demethylase and | human protein |
| | Lysine Hydrolase (JMJD6) | |
| 42 | Annexin A1 | human genomic |
| 43 | Annexin A2 | human genomic |
| 44 | Annexin A3 | human genomic |
| 45 | Annexin A4 | human genomic |
| 46 | Annexin A5 | human genomic |
| 47 | Annexin A6 | human genomic |
| 48 | Annexin A7 | human genomic |
| 49 | Annexin A8 | human genomic |
| 50 | Annexin A8L1 | human genomic |
| 51 | Annexin A9 | human genomic |
| 52 | Annexin A10 | human genomic |
| 53 | Annexin A11 | human genomic |
| 54 | Annexin A13 | human genomic |
| 55 | scFV of Bavitixumab | synthetic |
| 56 | Annexin Al (mRNA - NM_000700.3) | human mRNA |
| 57 | Annexin A2 (mRNA transcript 1 - NM_001002858.3) | human mRNA |
| 58 | Annexin A2 (mRNA transcript 2 - NM_001002857.2) | human mRNA |
| 59 | Annexin A3 (mRNA transcript - NM_005139.3) | human mRNA |
| 60 | Annexin A4 (mRNA transcript 1 - NM_001320698.2) | human mRNA |
| 61 | Annexin A4 (mRNA transcript 2 - NM_001153.5) | human mRNA |
| 62 | Annexin A4 (mRNA transcript 3 - NM_001320700.2) | human mRNA |
| 63 | Annexin A4 (mRNA transcript 4 - NM_001320702.2) | human mRNA |
| 64 | Annexin A5 (mRNA - NM_001154.4) | human mRNA |
| 65 | Annexin A6 (mRNA transcript 1 - NM_001155.5) | human mRNA |
| 66 | Annexin A6 (mRNA transcript 2 - NM_001193544.2) | human mRNA |
| 67 | Annexin A6 (mRNA transcript 3 - NM_001363114.2) | human mRNA |
| 68 | Annexin A7 (mRNA transcript 1 - NM_001156.5) | human mRNA |
| 69 | Annexin A7 (mRNA transcript 2 - NM_0044.4) | human mRNA |
| 70 | Annexin A7 (mRNA transcript 3 - NM_001320880.2) | human mRNA |
| 71 | Annexin A7 (mRNA transcript 4 - NM_001320879.2) | human mRNA |
| 72 | Annexin A8 (mRNA transcript 1 - NM_001271702.2) | human mRNA |
| 73 | Annexin A8 (mRNA transcript 2 - NM_001040084.3) | human mRNA |
| 74 | Annexin A8 (mRNA transcript 3 - NM_0012717.2) | human mRNA |
| 75 | Annexin A8L1 (mRNA transcript 1 - NM_001098845.3) | human mRNA |
| 76 | Annexin A8L1 (mRNA transcript 2 - NM_001278924.2) | human mRNA |
| 77 | Annexin A8L1 (mRNA transcript 3 - NM_001278923.2 | human mRNA |
| 78 | Annexin A9 (mRNA transcript - NM_0568.3) | human mRNA |
| 79 | Annexin 10 (mRNA transcript 1 - NM_007193.5) | human mRNA |
| 80 | Annexin A11 (mRNA transcript a - NM_001157.3) | human mRNA |
| 81 | Annexin A11 (mRNA transcript b - NM_001278409.2) | human mRNA |
| 82 | Annexin A13 (mRNA transcript a - NM_004306.4) | human mRNA |
| 83 | Annexin A13 (mRNA transcript b - NM_0010954.3) | human mRNA |
| 84 | IgG1 | human protein |
| 85 | Annexin V-Fc(IgG1) Fusion Protein with Secretion Signal | synthetic |
| 86 | Annexin V-Fc(IgG1) Fusion Protein | synthetic |
| 87 | AnnexinV-Fc(IgG1)-NKG2D Fusion Protein | synthetic |
| 88 | AnnexinV-scFv(OKT3) Fusion Protein with Secretion Signal | synthetic |
| 89 | AnnexinV-scFv (OKT3) Fusion Protein | synthetic |
| 90 | Annexin-scFv(OKT198A) Fusion Protein with Secretion | synthetic |
| | Signal | |
| 91 | Annexin-scFv(OKT198A) Fusion Protein | synthetic |
| 92 | AnnexinV-scFv(OKT198C) Fusion Protein with Secretion | synthetic |
| | Signal | |
| 93 | AnnexinV-scFv(OKT198C) Fusion Protein | synthetic |
| 94 | AnnexinV-scFv(OKT207A) Fusion Protein with Secretion | synthetic |
| | Signal | |
| 95 | AnnexinV-scFv(OKT207A) Fusion Protein | synthetic |
| 96 | AnnexinV-scFv(OKT3)-Hinge Fusion Protein with Secretion | synthetic |
| | Signal | |
| 97 | AnnexinV-scFv(OKT3)-Hinge Fusion Protein | synthetic |
| 98 | SIRPα Signal | synthetic |
| 99 | SIRPα-AnnexinV-Fc(IgG1) Fusion Protein with Secretion | synthetic |
| | Signal | |
| 100 | SIRPα-AnnexinV-Fc Fusion Protein | synthetic |
| 101 | AnnexinV-Fc-SIRPα Fusion Protein | synthetic |
| 102 | KLRK1 | human protein |
| 103 | KLRK1 (extracellular domain) | human protein |
| 104 | KLRK1 | human genomic |
| 105 | KLRK1 | human mRNA |
| 106 | Annexin V (annexin A5)-Fc(IgG1)-NKG2D Fusion Protein | synthetic |
| 107 | Secretion Signal | synthetic |
| 108 | Secretion Signal | synthetic |
| 109 | Flexible Linker - type 1 (v1) | synthetic |
| 110 | Flexible Linker - type 1 (v2) | synthetic |
| 111 | Flexible Linker - type 1 (v3) | synthetic |
| 112 | Flexible Linker - type 1 (v4) | synthetic |
| 113 | Flexible Linker - type 1 (v5) | synthetic |
| 114 | Flexible Linker - type 1 (v6) | synthetic |
| 115 | Flexible Linker - type 1 (v7) | synthetic |
| 116 | Flexible Linker - type 1 (v8) | synthetic |
| 117 | Flexible Linker (G) | synthetic |
| 118 | Flexible Linker (GG) | synthetic |
| 119 | Flexible Linker (GGG) | synthetic |
| 120 | Flexible Linker (GGGG) | synthetic |
| 121 | Flexible Linker (GGGGG) | synthetic |
| 122 | Flexible Linker (GGGGGG) | synthetic |
| 123 | Flexible Linker (GGGGGGG) | synthetic |
| 124 | Flexible Linker (GGGGGGGG) | synthetic |
| 125 | Rigid Linker (v1) | synthetic |
| 126 | Rigid Linker (v2) | synthetic |
| 127 | Rigid Linker (v3) | synthetic |
| 128 | Rigid Linker (v4) | synthetic |
| 129 | Rigid Linker (v5) | synthetic |
| 130 | Rigid Linker (v6) | synthetic |
| 131 | Rigid Linker (v7) | synthetic |
| 132 | Rigid Linker (v8) | synthetic |
| 133 | Whitlow Linker - type 1 (v1) | synthetic |
| 134 | Whitlow Linker - type 1 (v2) | synthetic |
| 135 | Whitlow Linker - type 1 (v3) | synthetic |
| 136 | Whitlow Linker - type 1 (v4) | synthetic |
| 137 | Whitlow Linker - type 1 (v5) | synthetic |
| 138 | Whitlow Linker - type 1 (v6) | synthetic |
| 139 | Whitlow Linker - type 1 (v7) | synthetic |
| 140 | Whitlow Linker - type 1 (v8) | synthetic |
| 141 | Whitlow Linker - type 2 (v1) | synthetic |
| 142 | Whitlow Linker - type 2 (v2) | synthetic |
| 143 | Whitlow Linker - type 2 (v3) | synthetic |
| 144 | Whitlow Linker - type 2 (v4) | synthetic |
| 145 | Whitlow Linker - type 2 (v5) | synthetic |
| 146 | Whitlow Linker - type 2 (v6) | synthetic |
| 147 | Whitlow Linker - type 2 (v7) | synthetic |
| 148 | Whitlow Linker - type 2 (v8) | synthetic |

### EXAMPLES

Most of the current paradigms for cancer therapy assume that PS-expressing tumor cells, either from exposure to internal or external stressors, are destined to die from apoptosis. But data provided herein showed that some of these PS-expressing tumor cells survived. As detailed herein, the compositions and methods described herein can significantly enhance the success rate of conventional cytotoxic cancer therapy.

The Examples that follow are illustrative of specific aspects of the invention, and various uses thereof. They set forth for explanatory purposes only and are not to be taken as limiting the invention.

### Example 1

### Examination of Survival of Phosphatidylserine-Positive Tumor Cells

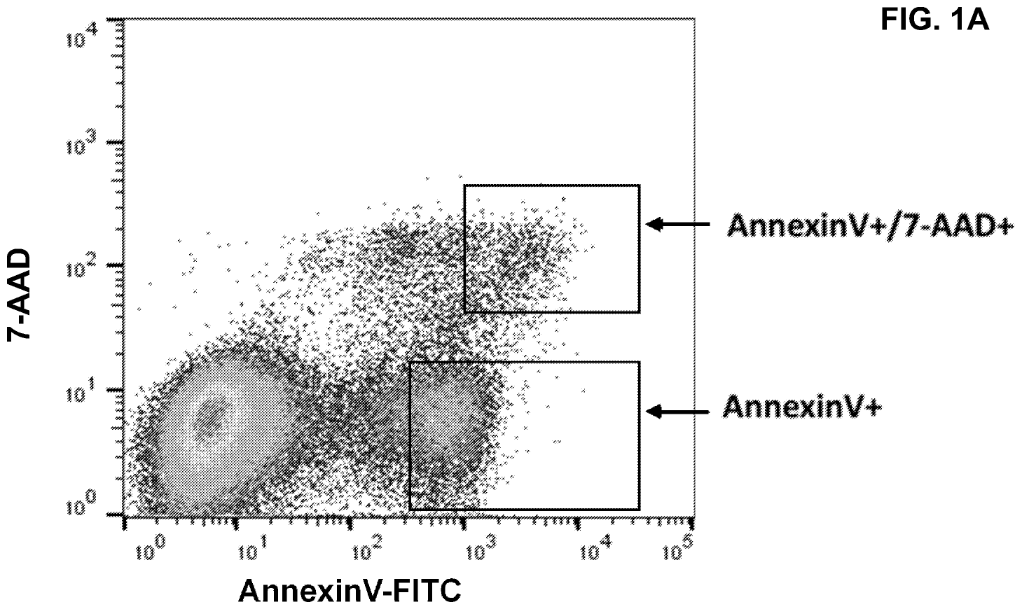

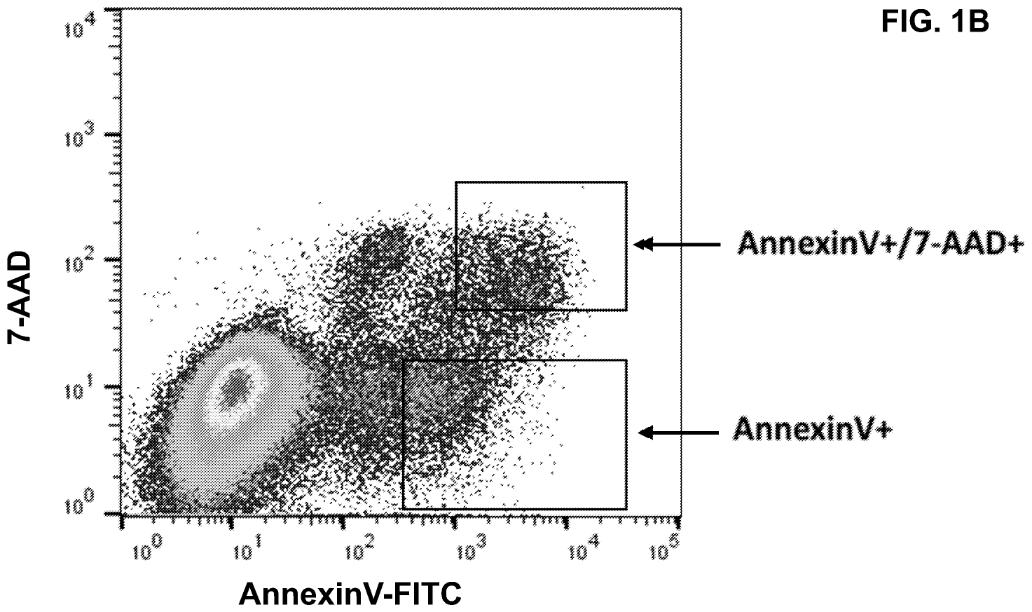

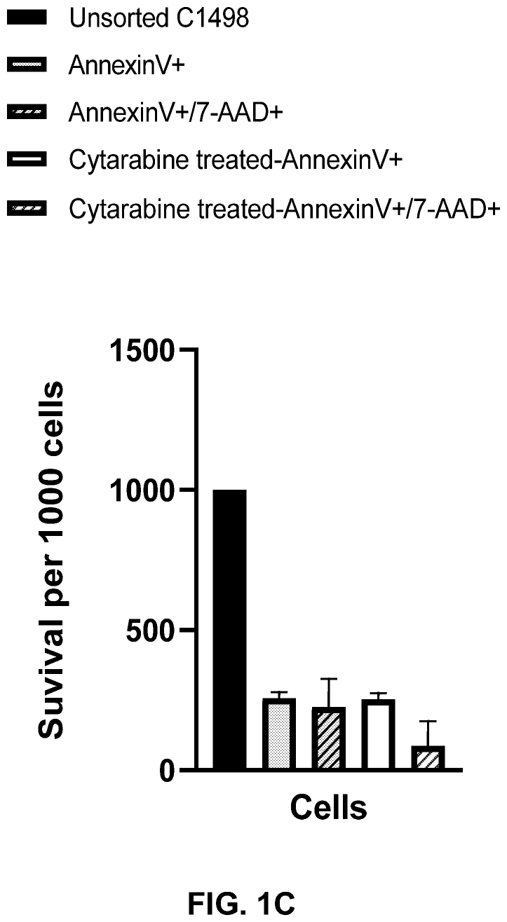

To determine whether cells expression high-level of PS expression on the cell surface are destined to die, the viability of c1498 murine AML (Acute Myeloid Leukemia) cells treated with cytarabine (part of a classical chemotherapy regimen for human AML) was examined. Specifically, whether cell surface PS-expressing acute myelocytic leukemia (AML) cells survived was examined. FACS was used to identify and sort cells with high cell surface PS-expression and the ability to survive was measured. FIG. 1A-FIG. 1B). In FIG. 1A, FACS sorting of untreated c1498 cells (in DMEM with 8% FBS). The rectangles indicate the sorted cells. In FIG. 1B, FACS sorting of c1498 cells treated with cytarabine (1 μM) for 24 hrs). The data indicated the following: (i) about 22-25% of untreated c1498 cells are either PS+7AAD-(7AAD is a fluorescent dye used to detect membrane integrity), early apoptotic cells, or PS+7AAD+, late apoptotic cells, remained alive and could proliferate in a robust manner (FIG. 1C), and (ii) about 25% or 8% of cytarabine-treated PS+7AAD-c1498 cells or PS+7AAD+ c1498 cells remained viable and could proliferate (FIG. 1C). These results indicated that a substantial proportion of PS+ cells (which the art presumed should be dead and/or destined to die), survived the initiation of the apoptotic process. The fact that these cells could survive indicated that new agents and/or treatment methods are needed to prevent tumor relapse. In FIG. 1C, about 1000 each of the sorted cells were placed into multi-well plates. When the cells reached sufficient numbers, the cells were counted at different time points to measure growth rates. The growth rates were then used to estimate the initial live cell numbers. The following formula was used to calculate the minimal initial live cell numbers: Minimal initial cell number=Total cell population/(growth rate*t(days to reach the current cell number).

### Example 2

### Annexin V-Fc Fusion Protein Mediated Phagocytosis of PS-Positive Tumor Cells

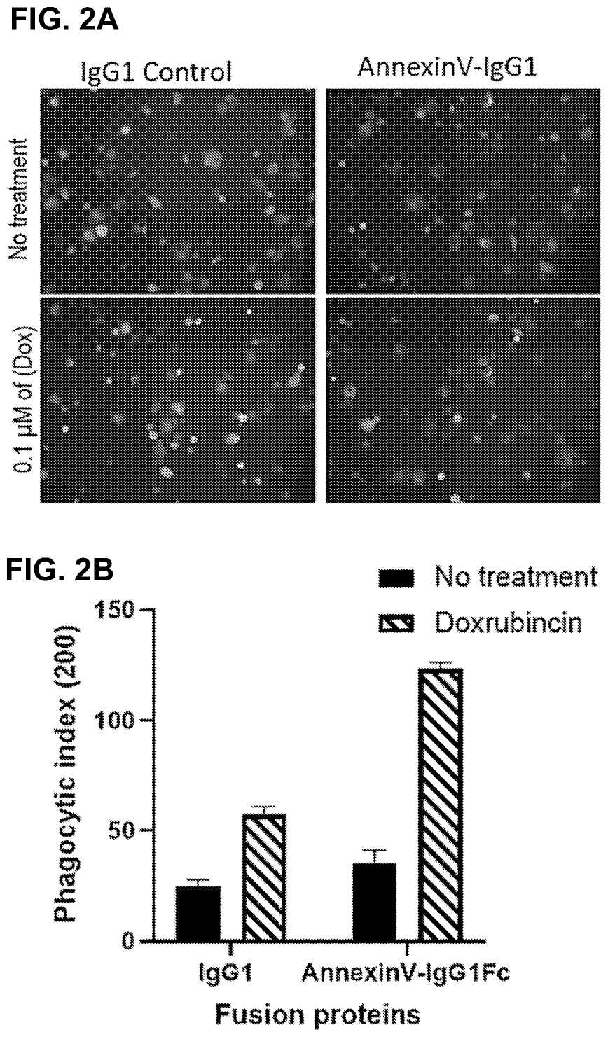

The ability of THP1-derived macrophages to phagocytose control or doxorubicin-treated c1498 cells was examined. FIG. 2A-FIG. 2B show that Annexin V-IgG1Fc fusion protein effectively boosted the phagocytosis of untreated or doxorubicin-treated c1498 cells. Representative fluorescence images of phagocytosis assay. THP1 cells transduced with mCherry were differentiated into macrophages following previously established procedures. The cells were then incubated with non-treated or doxorubicin-treated KG1a human AML cell line transduced with EGFP in the presence of either isotype control (IgG1) or AnnexinV-IgG1Fc. After 2 hrs of incubation, the macrophages and the KG1a cells they have engulfed were counted (FIG. 2A). Quantitative estimates of phagocytosis of KG1a cells by THP1-derived macrophages, the phagocytic index was calculated as: (Average number of KG1a cells engulfed per macrophage)×(percentage of macrophages which have engulfed KG1a cells)×200 (FIG. 2B).

### Example 3

Effective Treatment of c1498 AML Leukemia In Vivo by Use of Combined Chemotherapy and Annexin V-Fc Fusion Protein

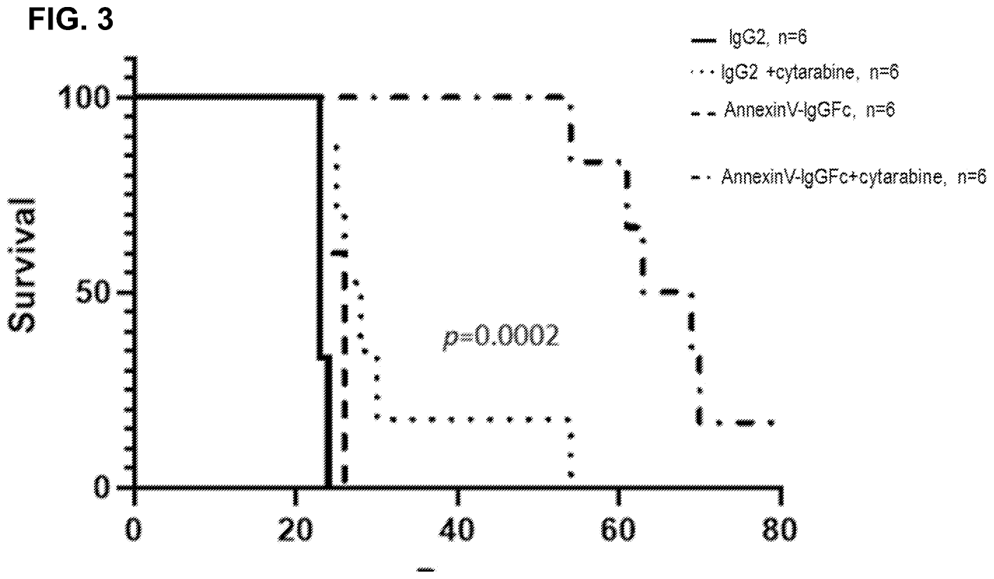

Whether Annexin V-IgG1Fc fusion protein can enhance the treatment of c1498 AML in syngeneic mice (C57BL/6) in combination with cytarabine, a commonly used drug for AML treatment, was examined. FIG. 3 shows that while neither cytarabine nor Annexin V-IgG1Fc fusion protein treatment significantly extended the survival of c1498-injected mice, the combination treatment significantly extended the median survival of host mice (>62 days for combination group vs, 27 days or less for the other groups). Here, about 2×10⁵ c1498 cells were injected into the tail vein of C57BL/6 mice. At 24 hrs after the injection, the mice were treated with 100 mg/kg cytarabine (injected i.p. every 12 hrs for 3 times). At 24 hrs after the last treatment, Annexin V-Fc was given i.p. 4 times at 72 hours intervals. The mice were then observed and sacrificed until they are moribund. FIG. 3 shows the survival of different mice after they were treated with different antibody and drug combinations.

### Example 4

### Effective Killing of Human AML (Acute Myeloid Leukemia) Cells by PBMC Cells in the Presence of Bispecific T-Cell Engagers (Bite) Targeting PS and CD3

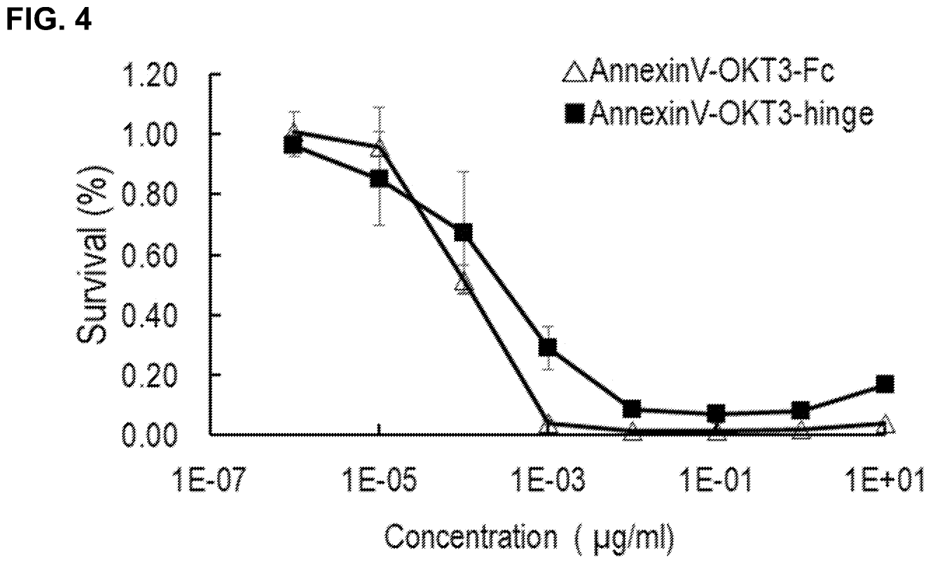

To determine whether a bi-specific T cell engager targeting phosphatidylserine (PS) could active T cells to kill AML cells, two dimerized BiTE proteins (AnnexinV-OKT3-Fc or AnnexinV-OKT3-hinge) were evaluated for efficacy to activate T cells to kill MV-4-11 human AML cells. FIG. 4 shows that both forms of BiTE could effectively mediate the killing of MV-4-11 cells in the presence of PBMC. Luciferase-labeled MV4-11 AML cells were mixed with PBMC cells at a ratio of 1:10. Bispecific proteins AnnexinV-scFv(OKT3)-Fc and AnnexinV-scFv(OKT3)-Hinge were added to the mixed cell population at different concentrations in 96-well plates. The survival of the MV4-11 cells were then quantified by measuring the luciferase activities of individual wells at 24 hrs post-bispecific protein addition (n=3).

### SUMMARY OF SPECIFIC EXAMPLES

As demonstrated herein, chimeric fusion proteins comprising a PS-binding domain increased the efficacy of chemotherapy. The Examples showed clearly that AML cells expressing PS were alive and proliferated in a robust manner (FIG. 1). These cells, previously thought to be destined to die and therefore irrelevant, were thus “survivors” that could propel the relapse of AML. Indeed, the data in FIG. 3 suggest that a fusion protein targeting PS could significantly enhance the therapeutic efficacy of chemotherapy provided strong support for our hypothesis. Similarly, the data in FIG. 4 provided additional support that PS-targeting bispecific engager effectively killed AML cells in the presence of T cells. These data provided strong support for the notion that direct targeting of PS on cancer cells could be an effective therapeutic strategy, which is against the prevailing view in the field currently.

What is claimed is:

1. A chimeric fusion protein, comprising: a phosphatidylserine (PS) binding domain operably linked to an immunostimulatory domain.

2. The chimeric fusion protein of claim 1, wherein the PS binding domain comprises Annexin A1 or the PS-binding core domain, Annexin A2, Annexin A3, Annexin A4, Annexin A5, Annexin A6, Annexin A7, Annexin A8, Annexin A8 Like 1, Annexin A9, Annexin A10, Annexin A11, Annexin A13, Adhesion G Protein Coupled Receptor B1 or the extracellular domain thereof, Apolipoprotein H, Coagulation Factor II, Coagulation Factor VII, Coagulation Factor IX, Coagulation Factor X, Growth Arrest Specific 6, Milk Fat Globule EGF And Factor V/VIII Domain Containing, Advanced Glycosylation End-Product Specific Receptor or the extracellular domain thereof, Protein S, Hepatitis A Virus Cellular Receptor 1 or the extracellular domain thereof, Hepatitis A Virus Cellular Receptor 2 or the extracellular domain thereof, T Cell Immunoglobulin and Mucin Domain Containing or the extracellular domain thereof, Protein Kinase C alpha or the C2 domain thereof, Synaptotagmin (Syt1) or the C2A domain thereof, Stabilin-1 or the extracellular domain thereof, or Stabilin-2 or the extracellular domain thereof.

3. The chimeric fusion protein of claim 1, wherein the PS binding domain comprises the single-chain variable domain of bavituximab, PGN632, P1, IS4, or CL1.

4. The chimeric fusion protein of claim 1, wherein the immunostimulatory domain comprises a single-chain antibody (scFv) to a human CD3 protein.

5. The chimeric fusion protein of claim 4, wherein the CD3 antibody comprises a OKT3 or humanized OKT3 antibody, a UCHT1 or humanized UCHT1 antibody, TRX4 (otlixizumab), foralumab, visilizumab, or tepilizumab.

6. The chimeric fusion protein of claim 1, wherein the PS-binding domain operably linked to an immunostimulatory domain is fused to an additional immunostimulatory domain.

7. The chimeric fusion protein of claim 6, wherein the additional immunostimulatory domain comprises an IgG1, IgG2, IgG3, or IgG4.

8. An isolated nucleic acid molecule encoding the chimeric fusion protein of any preceding claim.

9. A recombinant vector comprising the isolated nucleic acid molecule of claim 8.

10. The recombinant vector of claim 9, wherein the recombinant vector comprises a recombinant viral vector.

11. The recombinant vector of claim 10, wherein the recombinant viral vector comprises an adenovirus viral vector, an adeno-associated viral vector, a lentiviral viral vector, or a herpes simplex viral vector.

12. An antibody drug conjugate comprising the chimeric fusion protein of any one of claims 1-7.

13. An antibody drug conjugate comprising of the chimeric fusion protein where the PS-binding domain is operably linked to a Fc domain of IgG1, IgG2, IgG3, or IgG4.

14. A pharmaceutical formulation comprising the chimeric fusion protein of any one of claims 1-7, the isolated nucleic acid molecule of claim 8, the recombinant vector of any one of claims 9-11, and/or the antibody drug conjugate of claim 12 or claim 13; and one or more pharmaceutically acceptable carriers.

15. A method of treating a hematological cancer, the method comprising: administering to a subject in need thereof a therapeutically effective amount of the pharmaceutical formulation of claim 14, wherein, following administration, an immune response against PS-expressing hematological cancer cells is induced.

16. The method of claim 15, wherein the subject is treatment-naïve.

17. The method of claim 15, wherein the subject has received one or more anti-cancer therapies and/or treatments prior to the administering of the pharmaceutical formulation.

18. The method of claim 17, wherein the one or more anti-cancer therapies and/or treatments comprises endocrine therapy, radiotherapy, hormone therapy, gene therapy, thermal therapy, ultrasound therapy, or any combination thereof.

19. The method of claim 17, wherein the one or more anti-cancer therapies and/or treatments comprise one or more chemotherapeutic agents.

20. The method of claim 15, wherein the subject has one or more hematological cancers.

21. The method of claim 20, wherein the one or more hematological cancers comprise lymphoma, leukemia, and multiple myeloma.

22. The method of claim 20, wherein the one or more hematological cancers comprise acute myeloid leukemia (AML) or myelodysplastic syndrome.

23. The method of claim 15, wherein administering the pharmaceutical formulation comprises oral administration, intravenous administration, intratumor administration, intraperitoneal administration, intracranial administration, subcutaneous administration, intrathecal administration, or any combination thereof.

24. The method of claim 15, further comprising monitoring the subject for adverse effects.

25. The method of claim 24, wherein in the absence of adverse effects, the method further comprises continuing to administering to the subject the pharmaceutical formulation.

26. The method of claim 24, wherein in the presence of adverse effects, the method further comprises modifying one or more steps of the method.

27. The method of claim 17-18, further comprising repeating the administering to the subject of one or more additional anti-cancer therapies.

28. The method of claim 14, wherein the survivability of the subject is increased and/or improved.

29. The method of claims 18-19 or claim 27, wherein the method improves the efficacy of one or more anti-cancer therapies and/or treatments.

30. The method of claim 15, further comprising administering to the subject genetically modified T cells or NK cells or macrophages expressing a chimeric antigen receptor targeting phosphatidylserine (PS) or a pharmaceutical formulation thereof.
